(19) United States
(12) Patent Application Publication
(10) Pub. No.: US 2024/0263196 A1
(43) Pub. Date: Aug. 8, 2024

(54) Lipid Compositions and Methods for Delivery to Immune Cells

(71) Applicant: LIFE TECHNOLOGIES CORPORATION, Carlsbad, CA (US)

(72) Inventors: Jian LIU, Carlsbad, CA (US); Neha PARAYATH, Oceanside, CA (US); Evgenia VEROVSKAYA, Oceanside, CA (US)

(21) Appl. No.: 18/398,730

(22) Filed: Dec. 28, 2023

Related U.S. Application Data
(60) Provisional application No. 63/613,458, filed on Dec. 21, 2023.
(60) Provisional application No. 63/477,789, filed on Dec. 29, 2022.

(51) Int. Cl. C12N 15/88; C12N 9/22; C12N 15/11

(52) U.S. Cl. CPC C12N 15/88; C12N 9/22; C12N 15/11; C12N 2310/20

(57) ABSTRACT

Provided herein, are methods, compositions, and kits for introducing a payload to an immune cell. In aspects, a lipid composition is described, which includes at least one ionizable lipid compound having the structure I. In aspects, the methods and compositions are for introducing a nucleic acid payload into immune cells, for example T cells, dendritic cells, and NK cells.

### RELATED APPLICATIONS

This application claims the benefit of priority under 35 U.S.C. § 119(e) to U.S. Provisional Application No. 63/613,458 filed on Dec. 21, 2023 and to U.S. Provisional Application No. 63/477,789 filed on Dec. 29, 2022, the contents of which are hereby expressly incorporated herein by reference in their entirety as though fully set forth herein.

### SEQUENCE LISTING

The instant application contains a Sequence Listing which has been submitted electronically in ST.26 XML format and is hereby incorporated by reference in its entirety. Said XML document, created on Dec. 22, 2023, is named TP346833WO_SL.XML and is 734,000 bytes in size.

### TECHNICAL FIELD

The present invention is in the field of lipid compositions and formulations suitable for the delivery of one or more biologically active agents to a cell (e.g., an immune cell) and methods and kits for using the same.

### BACKGROUND

Lipid complexes, such as lipid nanoparticles, liposomes, etc) have been found to be useful as delivery agents to introduce macromolecule such as nucleic acids, protein, and small chemical molecules or pharmaceutically active molecules into cells and tissues in laboratory research and clinical settings.

There remains a need for improved compositions and methods for delivering biologically active payloads to immune system cells, including primary immune cells. Provided herein are compositions and methods which address this and other needs in the art.

### BRIEF SUMMARY

In one aspect, provided herein are methods for introducing a payload into an immune cell, including contacting an immune cell with a payload and a lipoplex comprising at least one ionizable at least one ionizable lipid compound, thereby introducing the payload into the immune cell; the at least one ionizable lipid compound having the structure I, or pharmaceutically acceptable salts thereof,

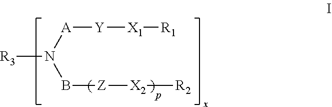

wherein:
each of X₁ and X₂ is a moiety independently selected from the group consisting of O, S, N—W and C—W, wherein W is selected from the group consisting of hydrogen and a C₁-C₂₀ hydrocarbon chain;
each of Y and Z is a moiety independently selected from the group consisting of CH—OH, C═O, C═S, S═O, and SO₂;
R₁, is selected from substituted or unsubstituted, branched or unbranched alkyl or alkenyl groups having between 3 and about 20 carbon atoms and between 0 and about 4 double bonds;
R₂, is selected from H or substituted or unsubstituted, branched or unbranched alkyl or alkenyl groups having between 3 and about 20 carbon atoms and between 0 and about 4 double bonds; each of A and B is selected from the group consisting of (CR₄R₅)ₙ, (CR₆R₇)ₘ, branched or unbranched alkyl groups having between 3 and about 20 carbon atoms, and
branched or unbranched alkenyl groups having between 3 and about 20 carbon atoms and having between 1 and about 4 double bonds;
each of R₄, R₅, R₆ and R₇ is a moiety independently selected from the group consisting of hydrogen, a cyclic or an acyclic, substituted or unsubstituted, branched or unbranched aliphatic group;
x is an integer independently having the value between 1 and 10, inclusively;
n is an integer independently having the value between 1 and 3, inclusively;
m is an integer independently having the value between 0 and 20, inclusively;
p is an integer independently having the value of 0 or 1; and
   R₃ is selected from

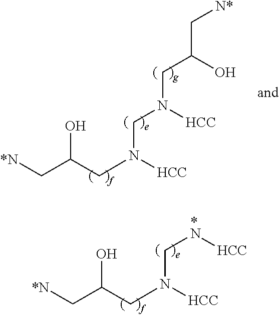

wherein “HCC” symbolizes a straight or branched alkyl, alkenyl or alkynyl hydrocarbon chain having up to about 20 carbon atoms; and each N* indicates the nitrogen atom N which is explicitly present in the above general structure I to which -A-Y—X₁—R₁, and —B—(Z—X₂)ₚ—R₂ are attached; and wherein each g, e and f is independently an integer between 1 and 6.

In another aspect, provided herein are lipoplex compositions for delivering a payload into an immune cell where the lipoplex comprises at least one ionizable lipid compound having structure I.

In some embodiments, the at least one ionizable lipid compound has the structure II, or pharmaceutically acceptable salts thereof,

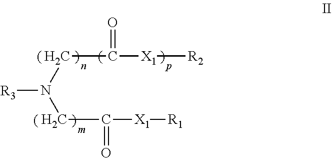

wherein:
each X₁ is independently selected from O or NH;
n is 0, 1, 2, or 3;
m is 1, 2, or 3;
p is 0 or 1, and when n=p=0, R₂ is H.

In some embodiments, e of structure I is 4, f and g of structure I are 1, and/or n and m of structure I are 2. In some embodiments, in structure I, e is 4, f and g are 1, and n and m are 1, 2, or 3. In some embodiments, the at least one ionizable lipid is selected from the group consisting of compounds 1-43.

In some embodiments, the immune cell is a T cell, a B cell, natural killer (NK) cell, a dendritic cell, or macrophage. In some embodiments, the immune cell is a helper T cell or a cytotoxic T cell. In some embodiments, the immune cell is a primary cell. In some embodiments, the immune cell is a human cell.

In some embodiments, the payload comprises at least one nucleic acid. In certain embodiments, the payload comprises an RNA molecule. In some embodiments, the RNA molecule includes mRNA, siRNA, shRNA, miRNA, self-replicating RNA (srRNA), self-amplifying RNA, stRNA, sgRNA, or combinations thereof. In some embodiments, the RNA molecule comprises more than one mRNA molecule.

In some embodiments, the nucleic acid or the RNA encodes a chimeric antigen receptor (CAR). In some embodiments, the RNA encodes a gene editing protein. In some embodiments, the RNA encodes a gene editing protein is selected from a Cas protein, a transcription activator-like effector nuclease (TALEN), a zinc finger nuclease, and a recombinase. In some embodiments, the nucleic acid payload comprises an sgRNA and an RNA encoding a gene editing protein.

In some embodiments, the payload comprises a gene editing protein. In some embodiments, the payload gene editing protein is selected from a Cas protein, a transcription activator-like effector nuclease (TALEN), a zinc finger nuclease, and a recombinase. In some embodiments, the payload comprises a ribonucleoprotein. In some embodiments, the payload ribonucleoprotein includes a Cas protein and a sgRNA.

In some embodiments, the at least one ionizable lipid of the lipoplex comprises a biodegradable linkage. In some embodiments, the at least one ionizable lipid in the lipoplex has a protonatable group with a pKa in the range of about 4 to about 8. In some embodiments, the at least one ionizable lipid in the lipoplex has a protonatable group with a pKa in the range of about 5 to about 7.5. In some embodiments, the at least one ionizable lipid in the lipoplex is positively charged at a pH below physiological pH.

In some embodiments, the lipoplex comprises at least one helper lipid. In some embodiments, the lipoplex comprises a helper lipid selected from the group consisting of comprises cholesterol, sterol, dioleoylphosphatidylethanolamine (DOPE), diphytanoylphosphatidylethanolamine (DPhPE), Lyso-PE (1-acyl-2-hydroxy-sn-glycero-3-phosphoethanolamine), Lyso-PC (1-acyl-3-hydroxy-sn-glycero-3-phosphocholine), distearoylphosphatidylcholine (DSPC), dioleoylphosphatidylcholine (DOPC), dipalmitoylphosphatidylcholine (DPPC), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dioleoyl-sn-glycero-3-phophoethanolamine (trans DOPE), or combinations thereof.

In some embodiments, the lipoplex comprises at least two different helper lipids. In some embodiments, at least one of the two helper lipids of the lipoplex is a sterol. In some embodiments, at least one of the two helper lipids of the lipoplex is a phospholipid.

In some embodiments, the lipoplex comprises a second ionizable lipid. In some embodiments, the lipoplex comprises at least one stabilizing agent. In some embodiments, the at least one stabilizing agent of the lipoplex is selected from a surfactant, a polymer conjugated lipid, and polyethylene glycol. In some embodiments, the at least one stabilizing agent of the lipoplex is a pegylated phospholipid. In some embodiments, the lipoplex comprises the at least one ionizable lipid compound having the structure I, at least one helper lipid and at least one stabilizing agent.

In some embodiments, the lipoplex comprise a fusion agent. In some embodiments, the lipoplex comprises a fusion agent which includes a polycationic nucleic acid binding moiety. In some embodiments, the lipoplex comprises a fusion agent which is a peptide. In some embodiments, the lipoplex comprises an endosomal release agent.

In some embodiments, the lipoplex comprises a nuclear localization peptide. In some embodiments, the lipoplex comprises a nuclear localization peptide which includes a polycationic nucleic acid binding moiety. In some embodiments, the lipoplex comprises a cell targeting agent. In some embodiments, the lipoplex comprises a cell targeting agent which includes a polycationic nucleic acid binding moiety. In some embodiments, the lipoplex comprises a cell targeting agent which is a cell surface ligand. In some embodiments, the lipoplex comprises an exosome or a biological material derived from or isolated from an exosome.

In some embodiments, the payload is a nucleic acid molecule, the ionizable lipid comprises a charge N and the nucleic acid molecule comprises a charge P, and the combination of the ionizable lipid and the nucleic acid contacting the cell comprises an N/P ratio from about 5 to 60. In some embodiments, the combination of the ionizable lipid and the nucleic acid payload contacting the cell comprises an N/P ratio from about 1 to 20.

In some embodiments, the at least one ionizable lipid is present in the lipoplex at a compositional molar ratio of about 0.10 to about 0.70. In some embodiments, the at least one helper lipid is present in the lipoplex at a compositional molar ratio of about 0.10 to about 0.90. In some embodiments, the at least one stabilizing agent is present in the lipoplex at a compositional molar ratio of about 0.005 to about 0.10.

In some embodiments of the provided methods, contacting of the immune cell with the payload and lipoplex occurs ex vivo or in vitro. In some embodiments, contacting of the immune cell with the payload and lipoplex occurs in vivo.

In some embodiments of the provided methods, the immune cell is in a population of immune cells during the contacting and the population of immune cells is cultured for a period of time following the contacting, where viability in the population of immune cells remains in excess of 60% for the period of time. In some embodiments, viability in the population of immune cells remains in excess of 70% for the period of time. In some embodiments, viability in the population of immune cells remains in excess of 80% for the period of time. In some embodiments, the population of immune cells is cultured for about 1 day to about 14 days following the contacting.

Other aspects of the invention are disclosed infra.

### BRIEF DESCRIPTION OF THE DRAWINGS

FIGS. 1A-1B are graphs depicting luminescent intensity (in Relative Light Units (RLU)) in activated T cells (FIG. 1A) and relative cell viability (FIG. 1B) following transfection with lipoplex compositions comprising luciferase-encoding mRNA. The T cells were transfected with either 100 ng or 300 ng RNA. Cell viability depicted is relative to that of Hela cell control set at 100%.

FIGS. 2A-2C are graphs depicting transfection efficiency (FIG. 2A), cell viability (FIG. 2B), and GFP fluorescence intensity (in Mean Fluorescence Intensities (MFI), FIG. 2C) following transfection with lipoplex compositions comprising GFP-encoding mRNA.

FIGS. 3A-3B are graphs depicting transfection efficiency (FIG. 3A) and cell viability (FIG. 3B) following transfection with LP1 in the presence or absence of ApoE4.

FIGS. 4A-4B are density plots depicting TCR knock-outs in activated T cells transfected with lipoplex compositions carrying sgRNA and Cas9-encoding mRNA. The cells were variously transfected with 100, 300, 400, or 500 ng RNAs and TCR expression on the cells assayed either 2 days post-transfection (FIG. 4A) or 5 days post-transfection (FIG. 4B). The control density plots were from untransfected T cells.

FIGS. 5A-5B are graphs depicting transfection efficiency (FIG. 5A) and cell viability (FIG. 5B) of mature dendritic cells following transfection with LP1 or MessengerMax compositions comprising GFP-encoding mRNA, compared to non-transfected control cells. Indicated below the x-axis for each is the amount of mRNA (50 or 100 ng) and the amount of lipid-based reagent used in the transfection reaction.

FIGS. 6A-6B are graphs depicting transfection efficiency (FIG. 6A) and cell viability (FIG. 6B) of NK cells following transfection with lipoplex composition LP1 comprising GFP-encoding mRNA (100 ng, 150 ng, or 200 ng).

### DETAILED DESCRIPTION

We have developed methods and compositions effective for delivering a macromolecule payload into immune cells. Surprisingly, it has been found that lipoplexes comprising an ionizable lipid having structure I are particularly effective in introducing a payload into immune cells, including for example primary T cells, dendritic cells, and natural killer (NK) cells. Payload-carrying lipoplexes comprising an ionizable lipid having structure I are also shown to have less cytotoxicity to the immune cells than payload-carrying lipoplexes comprising other cationic or ionizable lipids. The methods include combining at least one ionizable lipid having structure I with a payload and contacting an immune cell with the lipid-payload complex. Such complexes are easily prepared and are stable and therefor suitable for use in in vitro, ex vivo and in vivo applications, for example, delivery of therapeutic payloads (e.g., siRNA therapeutics, mRNA vaccine preparations, and the like), in cell therapy applications (e.g., delivery of chimeric antigen receptor (CAR) encoding nucleic acids or gene editing payloads), or the like.

In one aspect, methods are provided for introducing a payload into an immune cell using a lipoplex comprising at least one ionizable lipid having structure I complexed with the payload.

In another aspect, provided herein is a lipoplex composition for delivery of a payload to an immune cell, the lipoplex comprising at least one ionizable lipid having structure I. In some embodiments, the provided payload-lipoplex compositions are for modifying T cells isolated from patients. In some embodiments, the provided payload-lipoplex compositions are for modulating cells that have been engineered specifically to T cells or allogenic T cells. In some embodiments, the provided payload-lipoplex compositions are for modifying NK cells or modulating NK cells that have been engineered. In some embodiments, the provided payload-lipoplex compositions are for modifying dendritic cells or modulating dendritic cells that have been engineered.

In some embodiments, lipoplex compositions of the invention are used in preparation of a pharmaceutical composition for modifying or modulating immune cells. In some embodiments, lipoplex compositions of the invention are used in preparation of a pharmaceutical composition for modifying human T cells, such as allogenic T cells. In some embodiments, the pharmaceutical composition is used for modifying immune cells, wherein the immune cells are isolated from patients. In some embodiments, the pharmaceutical composition is used for modulating immune cells that have been engineered. In some embodiments, the pharmaceutical composition is used for modulating T cells isolated from patients or T cells that have been engineered specifically to T cells or allogenic T cells. In some embodiments, the pharmaceutical is used for modifying NK cells or modulating NK cells that have been engineered. In some embodiments, the pharmaceutical is used for modifying dendritic cells or modulating dendritic cells that have been engineered.

The following definitions are included for the purpose of understanding the present subject matter and for constructing the appended patent claims. The abbreviations used herein have their conventional meanings within the chemical and biological arts. While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

It is to be understood that the present invention is not limited to particular devices or biological systems, which may, of course, vary. Any methods, devices and materials similar or equivalent to those described herein can be used in the practice of this invention. The following definitions are provided to facilitate understanding of certain terms used frequently herein and are not meant to limit the scope of the present disclosure.

The terms used throughout this specification generally have their ordinary meanings in the art, within the context of the invention, and in the specific context where each term is used. Certain terms are discussed below, or elsewhere in the specification, to provide additional guidance to the practitioner in describing the general embodiments of the invention, as well as how to make and use them. It will be readily appreciated that the same thing can be said in more than one way. Consequently, alternative language and synonyms may be used for any one or more of the terms discussed herein, nor is any special significance to be placed upon whether or not a term is elaborated or discussed in greater detail herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term.

It is to be understood that, as used in this specification and the appended claims, the singular forms “a”, “an”, and “the” include singular and plural referents unless the content clearly dictates otherwise. Thus, for example, reference to “a lipid” includes one or more lipids. It is to be yet further understood that any terminology used herein is for the purpose of describing particular embodiments only, and is not intended to be limiting.

The term “about” when used in reference to numerical ranges, cutoffs, or specific values is used to indicate that the recited values may vary by up to as much as 25% from the listed value. As many of the numerical values used herein are experimentally determined, it should be understood by those skilled in the art that such determinations can, and often times will, vary among different experiments. The values used herein should not be considered unduly limiting by virtue of this inherent variation. The term “about” is used to encompass variations of 25% or less, variations of 20% or less, variations of 10% or less, variations of ±5% or less, variations of ±1% or less, variations of 0.5% or less, or variations of 0.1% or less from the specified value. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

Compounds of the present invention may exist in particular geometric or stereoisomeric forms. The present invention contemplates all such compounds, including cis- and toms-isomers, R- and S-enantiomers, diastereomers, (D)-isomers, (L)-isomers, the racemic mixtures thereof, and other mixtures thereof, as falling within the scope of the invention. Additional asymmetric carbon atoms is present in a substituent such as an alkyl group. All such isomers, as well as mixtures thereof, are intended to be included in this invention.

Isomeric mixtures containing any of a variety of isomer ratios is utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures. If, for instance, a particular enantiomer of a compound of the present invention is desired, it is prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless stated otherwise, the following terms, definitions, and abbreviations as used herein are intended to have the following meanings:

The term “protecting group,” as used herein, refers to a group that temporarily blocks a particular functional moiety, e.g., O, S, or N, is so that a reaction is carried out selectively at another reactive site in a multifunctional compound. A protecting group reacts selectively in good yield to give a protected substrate that is stable to the projected reactions, and the protecting group is selectively removable in good yield by readily available reagents that do not attack the other functional groups; the protecting group forms an easily separable derivative; and the protecting group has a minimum of additional functionality to avoid further sites of reaction.

As detailed herein, oxygen, sulfur, nitrogen, and carbon protecting groups is utilized. Non-limiting examples of exemplary hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl iV-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-niethoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxy acetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

For protecting 1,2- or 1,3-diols, non-limiting examples of exemplary protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

Non-limiting examples of exemplary amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-f-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocimiamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfnylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-l-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1, 4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl] amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1, 1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, NN′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl] amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMB S), benzylsulfonamide, trifluoromethylsulfonamide, and phenacylsulfonamide.

The term “substituted” whether used alone or is preceded by the term “optionally,” and substituents contained in formulae of this invention, refer to the replacement of hydrogen radicals in a given structure with the radical of a specified substituent. When more than one position in any given structure is substituted with more than one substituent selected from a specified group, the substituent is either the same or different at every position. The term “substituted” is inclusive of all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables are those that result in the formation of stable compounds.

The term “stable,” as used herein, refers to compounds which possess stability sufficient to allow manufacture and which maintain the integrity of the compound for a sufficient period of time to be detected and preferably for a sufficient period of time to be useful for the purposes detailed herein.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. The term “aliphatic” is inclusive of, but is not limited to, alkyl, alkenyl, alkynyl, cycloalkyl, cycloalkenyl, and cycloalkynyl moieties.

The term “alkyl” includes straight, branched and cyclic alkyl groups. An analogous convention applies to other generic terms such as “alkenyl” or “alkynyl.” The terms “alkyl,” “alkenyl” and “alkynyl” encompass both substituted and unsubstituted groups. The alkyl, alkenyl, and alkynyl groups employed in the invention contain 1-20 aliphatic carbon atoms. “Lower alkyl” is used to indicate those alkyl groups (cyclic, acyclic, substituted, unsubstituted, branched or unbranched) having 1-6 carbon atoms.

Exemplary aliphatic groups include, but are not limited to, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, and —CH₂-cyclohexyl moieties which are one or more substituents. Exemplary alkenyl groups include, but are not limited to, ethenyl, propenyl, butenyl and 1-methyl-2-buten-1-yl. Representative alkynyl groups include, but are not limited to, ethynyl, 2-propynyl (propargyl) and 1-propynyl.

The term “alkyl,” as used herein, refers to saturated, straight- or branched-chain hydrocarbon radicals derived from a hydrocarbon moiety containing between one and twenty carbon atoms by removal of a single hydrogen atom. Exemplary alkyl radicals include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The term “alkenyl,” as used herein, refers to a monovalent group derived from a hydrocarbon moiety having at least one carbon-carbon double bond by the removal of a single hydrogen atom. Exemplary alkenyl groups include, but are not limited to, ethenyl, propenyl, butenyl and 1-methyl-2-buten-1-yl.

The term “alkynyl,” as used herein refers to a monovalent group derived from a hydrocarbon having at least one carbon-carbon triple bond by the removal of a single hydrogen atom. Exemplary alkynyl groups include, but are not limited to, ethynyl, 2-propynyl (propargyl) and 1-propynyl, and the like.

The terms “alkoxy” and “thioalkyl,” as used herein refer to an alkyl group, as previously defined, attached to the parent molecule through an oxygen atom or through a sulfur atom, respectively. In certain embodiments, the alkyl, alkenyl, and alkynyl groups contain 1-20 alipahtic carbon atoms. Exemplary alkoxy groups, include but are not limited to, methoxy, ethoxy, propoxy, isopropoxy, n-butoxy, tert-butoxy, neopentoxy and n-hexoxy. Exemplary thioalkyl groups include, but are not limited to, methylthio, ethylthio, propylthio, isopropylthio and n-butylthio.

The term “alkylamino,” as used herein, refers to a group having the structure —NHR′, wherein R′ is aliphatic, as defined above, containing 1-20 aliphatic carbon atoms. Exemplary alkylamino groups include, but are not limited to, methylamino, ethylamino, n-propylamino, iso-propylamino, cyclopropylamino, n-butylamino, tert-butylamino, neopentylamino, n-pentylamino, hexylamino and cyclohexylamino.

The term “dialkylamino,” as used herein, refers to a group having the structure —NRR₁, wherein R and R₁ are each an aliphatic group, as defined herein, containing 1-20 aliphatic carbon atoms. R and R₁ is the same or different or is linked to form an aromatic or non-aromatic cyclic structure. Exemplary dialkylamino groups include, but are not limited to, dimethylamino, methyl ethylamino, diethylamino, methylpropylamino, di(n-propyl)amino, di(iso-propyi)amino, di(cyclopropyl)amino, di(n-butyl)amino, di(tert-butyl)amino, di(neopentyl)amino5 di(n-pentyl)amino, di(hexyl)amino and di(cyclohexyl)amino. Exemplary cyclic diaminoalkyl groups include, but are not limited to, aziridinyl, pyrrolidinyl, piperidinyl, morpholinyl, pyrrolyl, imidazolyl, 1,3,4-trianolyl and tetrazolyl.

The term “carboxylic acid,” as used herein, refers to a compound comprising a group of formula —COOH.

Some examples of substituents of the above-described aliphatic and other moieties of compounds of the invention include, but are not limited to aliphatic, heteroaliphatic, aryl, heteroaryl, arylalkyl, heteroarylalkyl, alkoxy, aryloxy, heteroalkoxy, heteroaryloxy, alkylthio, arylthio, heteroalkylthio, heteroarylthio, —F, —Cl, —Br, —I, —OH, —NO₂, —CN, —CF₃, —CH₂CF₃, —CHCl₂, —CH₂OH, —CH₂CH₂OH, —CH₂NH₂, —CH₂SO₂CH₃, —C(O)Rₓ, —CO₂(Rₓ), —CON(Rₓ)₂, —OC(O)Rₓ, —OCO₂Rₓ, —OCON(Rₓ)₂, —N(Rₓ)₂, —S(O)₂Rₓ and —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein is substituted or unsubstituted, branched or unbranched, cyclic or acyclic, and wherein any of the aryl or heteroaryl substituents described above and herein is substituted or unsubstituted.

The terms “aryl” and “heteroaryl,” as used herein, refer to stable mono- or polycyclic, heterocyclic, polycyclic, and polyheterocyclic unsaturated moieties having 3-14 carbon atoms, each of which is substituted or unsubstituted. Substituents include, but are not limited to, any of the substituents recited above for aliphatic moieties. The term “aryl” is inclusive of mono- or bicyclic carbocyclic ring systems having one or two aromatic rings including, but not limited to, phenyl, naphthyl, tetrahydronaphthyl, indanyl and The term “heteroaryl” is inclusive of cyclic aromatic radicals having from five to ten ring atoms, of which 1-3 ring atoms is selected from S, O, and N, and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl or isoquinolinyl.

Aryl and heteroaryl groups is unsubstituted or substituted, wherein substitution includes replacement of one, two, three, or more of the hydrogen atoms thereon independently with any one or more of the following moieties including, but not limited to aliphatic, heteroaliphatic, aryl, heteroaryl, arylalkyl, heteroarylalkyl, alkoxy, aryloxy, heteroalkoxy, heteroaryloxy, alkylthio, arylthio, heteroalkylthio, heteroarylthio, —F, —Cl, —Br, —I, —OH, —NO₂, —CN, —CF₃, —CH₂CF₃, —CHCl₂, —CH₂OH, —CH₂CH₂OH, —CH₂NH₂, —CH₂SO₂CH₃, —C(O)Rₓ, —CO₂(Rₓ), —CON(Rₓ)₂, —OC(O)Rₓ, —OCO₂Rₓ, —OCON(Rₓ)₂, —N(Rₓ)₂, —S(O)₂Rₓ and —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein is substituted or unsubstituted, branched or unbranched, cyclic or acyclic, and wherein any of the aryl or heteroaryl substituents described above and herein is substituted or unsubstituted.

The term “cycloalkyl,” as used herein, refers specifically to groups having three to seven, preferably three to ten carbon atoms. Suitable cycloalkyls include, but are not limited to cyclopropyl, cyclobutyl, cyclopentyl, cyclohexyl and cycloheptyl, which may optionally be substituted with substituents including, but not limited to aliphatic, heteroaliphatic, aryl, heteroaryl, arylalkyl, heteroarylalkyl, alkoxy, aryloxy, heteroalkoxy, heteroaryloxy, alkylthio, arylthio, heteroalkylthio, heteroarylthio, —F, —Cl, —Br, —I, —OH, —NO₂, —CN, —CF₃, —CH₂CF₃, —CHCl₂, —CH₂OH, —CH₂CH₂OH, —CH₂NH₂, —CH₂SO₂CH₃, —C(O)Rₓ, —CO₂(Rₓ), —CON(Rₓ)₂, —OC(O)Rₓ, —OCO₂Rₓ, —OCON(Rₓ)₂, —N(Rₓ)₂, —S(O)₂Rₓ and —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and is substituted or unsubstituted, branched or unbranched, cyclic or acyclic, and wherein any of the aryl or heteroaryl substituents described above and herein is substituted or unsubstituted.

The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties is branched, unbranched, cyclic or acyclic and include saturated and unsaturated heterocycles such as morpholino, pyrrolidinyl, etc. Heteroaliphatic moieties are substituted by independent replacement of one or more of the hydrogen atoms thereon with one or more moieties including, but not limited to aliphatic, heteroaliphatic, aryl, heteroaryl, arylalkyl, heteroarylalkyl, alkoxy, aryloxy, heteroalkoxy, heteroaryloxy, alkylthio, arylthio, heteroalkylthio, heteroarylthio, —F, —Cl, —Br, —I, —OH, —NO₂, —CN, —CF₃, —CH₂CF₃, —CHCl₂, —CH₂OH, —CH₂CH₂OH, —CH₂NH₂, —CH₂SO₂CH₃, —C(O)Rₓ, —CO₂(Rₓ), —CON(Rₓ)₂, —OC(O)Rₓ, —OCO₂Rₓ, —OCON(Rₓ)₂, —N(Rₓ)₂, —S(O)₂Rₓ and —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein is substituted or unsubstituted, branched or unbranched, cyclic or acyclic, and wherein any of the aryl or heteroaryl substituents described above and herein is substituted or unsubstituted.

The terms “halo” and “halogen,” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl and trifluoromethyl.

The term “heterocycloalkyl” or “heterocycle,” as used herein, refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms is optionally be oxidized, (iii) the nitrogen heteroatom may optionally be quaternized, and (iv) any of the above heterocyclic rings is fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl and tetrahydrofuryl. The term a “substituted” heterocycloalkyl or heterocycle group is utilized and as used herein, refers to a heterocycloalkyl or heterocycle group, as defined above, substituted by the independent replacement of one, two or three of the hydrogen atoms thereon with but are not to aliphatic, heteroaliphatic, aryl, heteroaryl, arylalkyl, heteroarylalkyl, alkoxy, aryloxy, heteroalkoxy, heteroaryloxy, alkylthio, arylthio, heteroalkylthio, heteroarylthio, —F, —Cl, —Br, —I, —OH, —NO₂, —CN, —CF₃, —CH₂CF₃, —CHCl₂, —CH₂OH, —CH₂CH₂OH, —CH₂NH₂, —CH₂SO₂CH₃, —C(O)Rₓ, —CO₂(Rₓ), —CON(Rₓ)₂, —OC(O)Rₓ, —OCO₂Rₓ, —OCON(Rₓ)₂, —N(Rₓ)₂, —S(O)₂Rₓ and —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein is substituted or unsubstituted, branched or unbranched, cyclic or acyclic, and wherein any of the aryl or heteroaryl substituents described above and herein is substituted or unsubstituted.

Exemplary non-limiting heterocyclic and aromatic heterocyclic groups that is included in the compounds of the invention include 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, A-(phenylmethyl)piperazine, 4-(1-phenylethyl)piperazine, 4-(1,1-dimethylethoxycarbonyl)piperazine, 4-(2-(bis-(2-propenyl) amino)ethyl)piperazine, A-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl) piperazine, 4-(2-nitrophenyl)piperazine, 4-(2-nitrophenyl)piperazine, 4-(2-phenylethyl)piperazine, A-(2-pyridyl)piperazine, 4-(2-pyrimidinyl)piperazine, 4-(2,3-dimethylphenyl)piperazine, 4-(2,4-difluorophenyl) piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The term “carbocycle,” as used herein, refers to an aromatic or non-aromatic ring in which each atom of the ring is a carbon atom.

The term “independently selected” is used herein to indicate that the groups is identical or different.

As used herein, the term “labeled” is intended to mean that a compound has at least one element, isotope, or chemical compound attached to enable the detection of the compound by using a radioactive or heavy isotope label, or an immune label such as an antibody or antigen or a label derived from a colored, luminescent, phosphorescent, or fluorescent dye. Photoaffinity labeling employing, for example, o-, m- and p-azidobenzoyls, substituted with one or more halogen moieties, including, but not limited to 4-azido-2,3,5,6-tetrafluorobenzoic acid, is utilized for the direct elucidation of intermolecular interactions in biological systems.

The term “animal,” as used herein, may refer to humans as well as non-human animals, including, for example, mammals (e.g., a rodent, a mouse, a rat, a rabbit, a monkey, a dog, a cat, a primate, or a pig), birds, reptiles, amphibians, and fish.

The term “cell” generally refers to eukaryotic cells of any type and from any source. Types of eukaryotic cells include immune, epithelial, fibroblastic, neuronal, hematopoietic cells and the like from primary cells, tumor cells or immortalized cell lines. Sources of such cells include any animal such as human, canine, mouse, hamster, cat, bovine, porcine, monkey, ape, sheep, and fish.

“Delivery” is used to denote a process by which a desired payload compound is transferred to a target cell such that the desired payload compound is ultimately located inside the target cell or in, or on, the target cell membrane. In some uses of the compounds of the invention, the desired payload compound is not readily taken up by the target cell and delivery via lipoplexes or transfection complexes is a means for delivering the desired compound to the appropriate cellular compartment within a cell. In certain uses, including under in vivo conditions, delivery to a specific target cell type is preferable and can be facilitated by lipid compounds of the invention.

As used herein, the term “lipoplex” is a generic term that includes lipid nanoparticles, liposomes of all types, both unilamellar and multilamellar, as well as vesicles, micelles, exosomes, microvesicles and more amorphous aggregates. A lipoplex can form a lipid-payload complex when contacted with a suitable payload agent. The term “lipoplex” is generally used herein to refer to a “naked” delivery or transfection complex, i.e., a delivery or transfection complex that generally lacks a payload agent to be delivered to a cell or to a tissue in vitro, ex vivo, or in vivo.

“Kit” refers to transfection or payload delivery kits which include one or more of the compounds of the present invention or mixtures thereof. Such kits may comprise a carrying means being compartmentalized to receive in close confinement one or more container means such as vials, test tubes and the like. Each of such container means comprises components or a mixture of components needed to perform transfection. Such kits may include one or more components selected from one or more ionizable lipid compounds of the present invention, helper lipids, stabilizing agents, payloads for delivery (for example, nucleic acid, proteins, etc.), cells, lipoplex-forming compounds, transfection enhancers, biologically active substances, etc.

The term “associated with”, when used in the context of molecular interactions, refers to two entities linked by a direct or indirect covalent or non-covalent interaction, such as hydrogen bonding, van der Waals interactions, hydrophobic interactions, magnetic interactions, electrostatic interactions, etc.

The term “biocompatible,” as used herein refers to compounds that are not toxic to cells. Compounds are biocompatible if their addition to cells in vitro results in less than or equal to 20% cell death, and their administration in vivo does not induce inflammation or other such adverse effects.

The term “biodegradable,” as used herein, refers to compounds that, when introduced into cells, are broken down into components that the cells can either reuse or dispose of without significant toxic effect on the cells (i.e., fewer than about 20% of the cells are killed when the components are added to cells in vitro). The components do not induce inflammation or other adverse effects in vivo. The chemical reactions relied upon to break down the biodegradable compounds are typically uncatalyzed.

The term “effective amount,” as used herein with respect to an active agent, refers to the amount necessary to elicit the desired biological response. The effective amount of an agent or device may vary depending on such factors as the desired biological endpoint, the agent to be delivered, the composition of the encapsulating matrix, the target tissue, etc. Delivery of an “effective amount of a molecule” is the delivery of the molecule into a cell in sufficient amounts so that the molecule elicits a biological response, for example, modulating the expression of one or more genes in the cell. In some embodiments, an effective amount of a molecule is delivered to a cell such that an amelioration or improvement in a disease, condition, or disorder related to the cell can be obtained. For example, delivery of an “effective amount of siRNA” or an “effective amount or RNAi” is the delivery of siRNA or other RNAi into a cell in sufficient amounts to cause a reduction in expression of the target gene in the cell.

The terms “biologically active agent”, “bioactive agents” or the like, generally refers to a composition, complex, compound or molecule which has a biological effect or that modifies, causes, promotes, enhances, blocks or reduces a biological effect, or that enhances or limits the production or activity of, reacts with and/or binds to a second molecules which has a biological effect. The second molecule can, but need not be, an endogenous molecule (e.g., a molecule, such as a protein or nucleic acid, normally present in the target cell). A biological effect may be, but is not limited to, one that stimulates or causes an immunoreactive response; one that impacts a biological process in a cell, tissue or organism (e.g., in an animal); one that imparts a biological process in a pathogen or parasite; one that generated or causes to be generated a detectable signal; one that regulates the expression of a protein or polypeptide; one that stops or inhibits the expression of a protein or polypeptide; or one that causes or enhances the expression of a protein or polypeptide. Biologically active compositions, complexes, compounds or molecules may be used in investigative, therapeutic, prophylactic and diagnostic methods and compositions and generally act to cause.

The term “cationic lipid” refers to any cationic lipids which may be used for transfection and which under physiological conditions possess at least one positive charge. While it is to be understood that certain of the amine-containing transfection agents that form the basis of the present disclosure also exist as cations under physiological conditions, the term is also extended without limitation to any cationic helper lipids that may be used to co-formulate transfection complexes as described herein.

The term “polycationic nucleic acid binding moiety” as used herein refers to a moiety containing multiple positive charges at physiological pH that allow the moiety to bind a negatively charged nucleic acid. A polycationic nucleic acid binding moiety may be linked to, for example, a cell surface ligand, a fusion agent, and/or a nuclear localization peptide. The linkage may be covalent. Suitable polycationic nucleic acid binding moieties include polyamines and polybasic peptides containing, for example, multiple lysine, ornithine, or histidine residues.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. “Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long. A “fusion protein” refers to a chimeric protein encoding two or more separate protein sequences that are recombinantly expressed or chemically synthesized as a single moiety.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection.

The term “lysis agent” or “endosomal release agent” as used herein refers to a molecule, compound, protein or peptide which is capable of breaking down an endosomal membrane and freeing the DNA transporter into the cytoplasm of the cell. This term includes but is not limited to viruses, synthetic compounds, lytic peptides, or derivatives thereof. The term “lytic peptide” refers to a chemical grouping which penetrates a membrane such that the structural organization and integrity of the membrane is lost. As a result of the presence of the lysis agent, the membrane undergoes lysis, fusion or both. Examples of lysis agents/endosomal release agents include choroquine, polamines and polyamidoamines. Suitable agents are described in, for example, ei and Buyanova, Bioconjugate Chem, 30:273-283 (2009) and Juliano, Nucleic Acid Therapeutics, 28:166-177 (2018).

The term “surface ligand” or “cell surface ligand” refers to a chemical compound or structure which will bind to a surface receptor of a cell. The term “cell surface receptor” as used herein refers to a specific chemical grouping on the surface of a cell to which the ligand can attach. Cell surface receptors can be specific for a particular cell, i.e., found predominantly in one cell rather than in another type of cell (e.g., T cell receptors are specific for T cells). The receptor facilitates the internalization of the ligand and attached molecules. A cell surface receptor includes but is not limited to a folate receptor, biotin receptor, lipoic acid receptor, low-density lipoprotein receptor, asialoglycoprotein receptor, insulin-like growth factor type II/cation-independent mannose-6-phosphate receptor, calcitonin gene-related peptide receptor, insulin-like growth factor I receptor, nicotinic acetylcholine receptor, hepatocyte growth factor receptor, endothelin receptor, bile acid receptor, bone morphogenetic protein receptor, cartilage induction factor receptor or glycosylphosphatidylinositol (GPI)-anchored proteins (e.g., 0-adrenergic receptor, T-cell activating protein, Thy-1 protein, GPI-anchored 5′ nucleotidase). These are nonlimiting examples.

A “receptor” is a molecule to which a ligand binds specifically and with relatively high affinity. A receptor is usually a protein or a glycoprotein, but may also be a glycolipid, a lipidpolysaccharide, a glycosaminoglycan or a glycocalyx. For purposes of this disclosure, epitopes to which an antibody or its fragments binds is construed as a receptor since the antigen:antibody complex undergoes endocytosis. Furthermore, surface ligand includes anything which is capable of entering the cell through cytosis (e.g. endocytosis, potocytosis, pinocytosis).

As used herein, the term “ligand” refers to a chemical compound or structure which will bind to a receptor. This includes but is not limited to ligands such as asialoorosomucoid, asialoglycoprotein, lipoic acid, biotin, apolipoprotein E sequence, insulin-like growth factor II, calcitonin gene-related peptide, thymopoietin, hepatocyte growth factor, endothelin-1, atrial natriuretic factor, RGD-containing cell adhesion peptides and the like. The ligand may also be a plant virus movement protein or peptide derived from such a protein. Suitable peptides and proteins are described, for example, in U.S. Pat. No. 10,538,784, the contents of which are hereby incorporated by reference in their entirety.

One skilled in the art will readily recognize that a ligand chosen will depend on which receptor is being bound. Since different types of cells have different receptors, this provides one method of targeting nucleic acid to specific cell types, depending on which cell surface ligand is used. Thus, use of a cell surface ligand may depend on the targeted cell type.

Provided herein are lipoplex compositions for delivery of a payload to an immune cell, the lipoplex comprising at least one ionizable lipid having structure I.

As used herein, the term “lipoplex” is a generic term that includes lipid nanoparticles, liposomes of all types, both unilamellar and multilamellar, as well as vesicles, micelles, exosomes, microvesicles and more amorphous aggregates. A lipoplex can form a lipid-payload complex when contacted with a suitable payload agent. The term “lipoplex” is generally used herein to refer to a “naked” delivery or transfection complex, i.e., a delivery or transfection complex that generally lacks a payload agent to be delivered to a cell or to a tissue in vitro or in vivo.

The ionizable lipid compositions provided herein encompass complexes in the form of lipid nanoparticles, liposomes (e.g., lipid vesicles) and lipoplexes. As used herein, the term “liposome” encompasses any compartment enclosed by a lipid bilayer. The term liposome includes unilamellar vesicles which are comprised of a single lipid bilayer and generally have a diameter in the range of about 20 to about 400 nm. Liposomes can also be multilamellar having a diameter in the range of approximately 1 μm to approximately 10 μm. Multilamellar liposomes may consist of several (anywhere from two to hundreds) unilamellar vesicles forming one inside the other in diminishing size, creating a multilamellar structure of concentric phospholipid spheres separated by layers of water. Alternatively, multilamellar liposomes may consist of many smaller nonconcentric spheres of lipid inside a large liposome. In embodiments, liposomes include multilamellar vesicles (MLV), large unilamellar vesicles (LUV), and small unilamellar vesicles (SUV). In some embodiments, the lipoplex compositions include liposomes which contain an ionizable lipid having structure I and helper lipid(s). In some embodiments, the lipoplex compositions include liposomes which contain an ionizable lipid having structure I and helper lipid(s), along with a payload.

In some embodiments, the lipoplex compositions include lipid nanoparticles (LNPs). LNP composition are typically sized on the order of micrometers or small and may include a lipid bilayer. In some embodiments, the lipid nanoparticle composition comprises a lipid formulation including at least one ionizable lipid having structure I, wherein the size is from about 20 nm to about 1 μm. In some embodiments, the lipid nanoparticle composition comprises a lipid formulation including at least one ionizable lipid having structure I, along with a payload.

Exemplary payloads for delivery to an immune cell via the provided lipoplex compositions include nucleic acid molecules, protein molecules and/or other bioactive agents. In some embodiments, the payload for delivery to an immune cell is a therapeutic agent or a diagnostic agent.

Examples of nucleic acid therapeutics include but are not limited to antisense oligonucleotides, ribozymes, microRNA, mRNA, ribozyme, tRNA, tracrRNA, sgRNA, snRNA, siRNA, shRNA, ncRNA, miRNA, mRNA, pre-condensed DNA, plasmid DNA (pDNA) or an aptamer. Exemplary nucleic acid payloads are used to silence genes (with for example siRNA), express genes (with for example mRNA), edit genomes (with for example CRISPR/Cas9), and program or reprogram cells for return to a subject (for example ex vivo cell therapy to program or reprogram immune cells for cancer therapy including autologous transfer or allogenic transfer to a subject in need thereof).

In some embodiments, the nucleic acid payload is an RNA molecule. For example, the RNA molecule comprises mRNA, siRNA, shRNA, miRNA, self-replicating RNA (srRNA), self-amplifying RNA, stRNA, sgRNA, or combinations thereof. In some embodiments, the RNA molecule includes more than one mRNA molecule (e.g., at least 2 mRNA molecules, at least 3 mRNA molecules, at least four mRNA molecules, or at least 5 mRNA molecules). In some embodiments, the RNA molecule includes at least one sgRNA. In still some examples, the payload includes an RNA molecule, and the RNA molecule includes sgRNA, a crRNA, a tracrRNA, or combinations thereof. In other embodiments, the nucleic acid of the lipid complex composition molecule includes an sgRNA molecule and an mRNA molecule.

In some embodiments, the payload includes a gene editing reagent (or a gene editing composition), and the gene editing reagent includes a gene editing protein, an RNA molecule, and/or a ribonucleoprotein (RNP). In various examples, the gene editing protein includes a zinc finger nuclease (ZFN), a transcription activator-like effector nuclease (TALEN), a Cas protein, a MegaTal, a Cre recombinase, a Hin Recombinase, or a Flp recombinase. In some embodiments, the RNA molecule includes sgRNA, a crRNA, and/or a tracrRNA. Accordingly, in some embodiments, the payload for delivery to an immune cell includes an RNP and an sgRNA. In some embodiments, the RNP can include a Cas protein and a sgRNA, a crRNA or a tracrRNA.

In other embodiments, the RNA may encode a gene editing protein (e.g., an RNA encoding a ZFN, TALEN, Cas protein, Cre recombinase, etc). Accordingly, in some embodiments, the payload for delivery to an immune cell includes an RNA encoding a gene editing protein and an sgRNA. In some embodiments, the payload can include an RNA encoding a Cas protein and a sgRNA, a crRNA or a tracrRNA.

In some embodiments, the nucleic acid payload is a single-stranded molecule. In some embodiments, the payload may include DNA. In still other embodiments, the DNA payload may be a plasmid DNA or linear DNA.

In certain embodiments, the gene editing payload induces single-strand or double-strand breaks in DNA within the cells. In some embodiments the gene editing reagent (or gene editing composition) further comprises a repair template polynucleotide. In various embodiments, the repair template comprises (a) a first flanking region comprising nucleotides in a sequence complementary to about 40 to about 90 base pairs on one side of the single or double strand break and a second flanking region comprising nucleotides in a sequence complementary to about 40 to about 90 base pairs on the other side of the single or double strand break; or (b) a first flanking region comprising nucleotides in a sequence complementary to at least about 20, 25, 30, 35, 40, 45, 50, 60, 70, 80, or 90 base pairs on one side of the single or double strand break and a second flanking region comprising nucleotides in a sequence complementary to at least about 20, 25, 30, 35, 40, 45, 50, 60, 70, 80, or 90 base pairs on the other side of the single or double strand break. Non-limiting descriptions relating to gene editing (including repair templates) using the CRISPR-Cas system are discussed in Ran et al. (2013) Nat Protoc. 2013 November; 8(11): 2281-2308, the entire content of which is incorporated herein by reference. Embodiments involving repair templates are not limited to those comprising the CRISPR-Cas system.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In aspects of the invention, nickases may be used for genome editing via homologous recombination.

In certain embodiments, the payload may include a Cas9 nickase used in combination with guide sequence(s), e.g., two guide sequences, which target respectively sense and antisense strands of the DNA target. This combination allows both strands to be nicked and used to induce NHEJ.

In some embodiments, the nucleic acid payload of the lipoplex composition encodes for an immunogen. In examples, the lipoplex composition comprising a nucleic acid payload induces an immune response in a subject to the nucleic acid-encoded protein of the lipoplex.

In some embodiments, a nucleic acid payload encodes a genetically engineered receptor that specifically binds to a ligand, such as a ligand expressed on a cancer cell, a molecule involved in a metabolic pathway, or functional portions thereof. Accordingly, in some embodiments, a nucleic acid payload encodes a chimeric antigen receptor (CAR) for delivery to an immune cell. In some embodiments, a CAR-encoding nucleic acid payload is delivered to an immune cell, such as a T cell or NK cell, via a lipoplex comprising at least one ionizable lipid having structure I. Such transfected cells may be used in CAR-T or CAR-NK cell therapies.

As used herein the term “ionizable lipid” refers to a lipid having one or more functional groups that can reversibly be ionized (protonated or deprotonated) depending on the pH of the medium containing the lipid. The functional group may be basic, such as an amino function, or may be acidic, such as a carboxylic acid moiety. The skilled artisan will be aware that other ionizable functional groups also may be used. An ionizable lipid may contain both basic and acid moieties. Advantageously, an ionizable lipid carries an overall positive charge at physiological pH. Ionizable lipids include, for example, amine-containing lipids that can be readily protonated.

In one aspect, lipoplex compositions provided herein comprise at least one ionizable lipid having the general structure I, or pharmaceutically acceptable salts thereof:

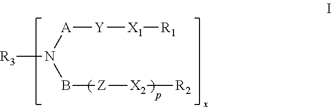

wherein:
   each of X₁ and X₂ is a moiety independently selected from the group consisting of O, S, N—W and C—W, wherein W is selected from the group consisting of hydrogen and a C₁-C₂₀ hydrocarbon chain;
each of Y and Z is a moiety independently selected from the group consisting of CH—OH, C═O, C═S, S═O, and SO₂;
R₁, is selected from substituted or unsubstituted, branched or unbranched alkyl or alkenyl groups having between 3 and about 20 carbon atoms and between 0 and about 4 double bonds;
R₂, is selected from H or substituted or unsubstituted, branched or unbranched alkyl or alkenyl groups having between 3 and about 20 carbon atoms and between 0 and about 4 double bonds;
each of A and B is selected from the group consisting of (CR₄R₅)ₙ, (CR₆R₇)ₘ, branched or unbranched alkyl groups having between 3 and about 20 carbon atoms, and
branched or unbranched alkenyl groups having between 3 and about 20 carbon atoms and having between 1 and about 4 double bonds;
each of R₄, R₅, R₆ and R₇ is a moiety independently selected from the group consisting of hydrogen, a cyclic or an acyclic, substituted or unsubstituted, branched or unbranched aliphatic group;
x is an integer independently having the value between 1 and 10, inclusively;
n is an integer independently having the value between 1 and 3, inclusively;
m is an integer independently having the value between 0 and 20, inclusively;
p is an integer independently having the value of 0 or 1; and
R₃ is selected from

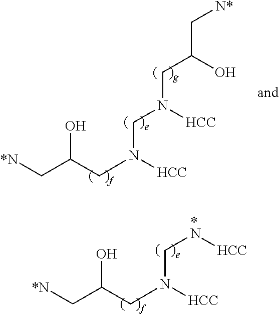

wherein “HCC” symbolizes a straight or branched alkyl, alkenyl or alkynyl hydrocarbon chain having up to about 20 carbon atoms; and each N* indicates the nitrogen atom N which is explicitly present in the above general structure I to which -A-Y—X₁—R₁, and —B—(Z—X₂)ₚ—R₂ are attached; and wherein each g, e and f is independently an integer between 1 and 6.

In some embodiments, each of A and B in structure I is independently branched or unbranched alkenyl groups having between 3 and about 20 carbon atoms and having between 1 and about 4 double bonds.

In some embodiments, each of A and B in structure I is independently propenyl, butenyl or 1-methyl-2-buten-1-yl.

In some embodiments, where m=p=0 in structure I, then R₂ is hydrogen. In some embodiments, each of R1 and R2 in structure I is independently any of substituted or unsubstituted, branched or unbranched alkyl or alkenyl groups having between 8 and about 18 carbon atoms and between 0 and 2 double bonds. In some embodiments, each of R₁ and R₂ in structure I are the same.

In some embodiments, compounds the structure I may have each of R₄, R₅, R₆ and R₇ being hydrogen, each of Y and Z being C═O, and each of X₁ and X₂ being the same. Such compounds are represented by the structure II (which is a sub-genus of the compound the general structure I):

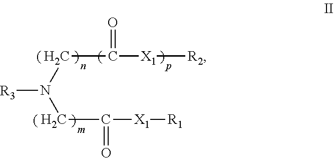

wherein when n=p=0, R₂ is H.
X₁ is a moiety independently selected from the group consisting of O, S, N—W and C—W, wherein W is selected from the group consisting of hydrogen and a C₁-C₂₀ hydrocarbon chain;
R₁, is selected from substituted or unsubstituted, branched or unbranched alkyl or alkenyl groups having between 3 and about 20 carbon atoms and between 0 and about 4 double bonds;
R₂, is selected from H or substituted or unsubstituted, branched or unbranched alkyl or alkenyl groups having between 3 and about 20 carbon atoms and between 0 and about 4 double bonds;
m is an integer independently having the value between 1 and 3, inclusively;
n is an integer independently having the value between 0 and 20, inclusively;
p is an integer independently having the value of 0 or 1, and when n=p=0, R₂ is H; and
R₃ is selected from

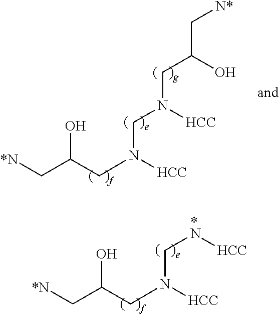

wherein “HCC” symbolizes a straight or branched alkyl, alkenyl or alkynyl hydrocarbon chain having up to about 20 carbon atoms; and each N* indicates the nitrogen atom N which is explicitly present in the above structure II to which —(CH₂)ₘ—CO—X₁—R₁, and —(CH₂)ₙ—(CO—X₁)ₚ—R₂ are attached; and wherein each g, e and f is independently an integer between 1 and 6.

In some embodiments, each of R₁ and R₂ in structure II are the same. In some embodiments, each of R₁ and R₂ in structure II is independently any of substituted or unsubstituted, branched or unbranched alkyl or alkenyl groups having between 8 and about 18 carbon atoms and between 0 and 2 double bonds.

According to some embodiments, lipoplex compositions provided herein comprise one or more specific compounds that are species within either the general structure I or the structure II, or both. Non-limiting examples of such specific compounds are any of the following lipids 1-43, or any isomer of each of compounds 1-43, or any combination of isomers for each of compounds 1-43:

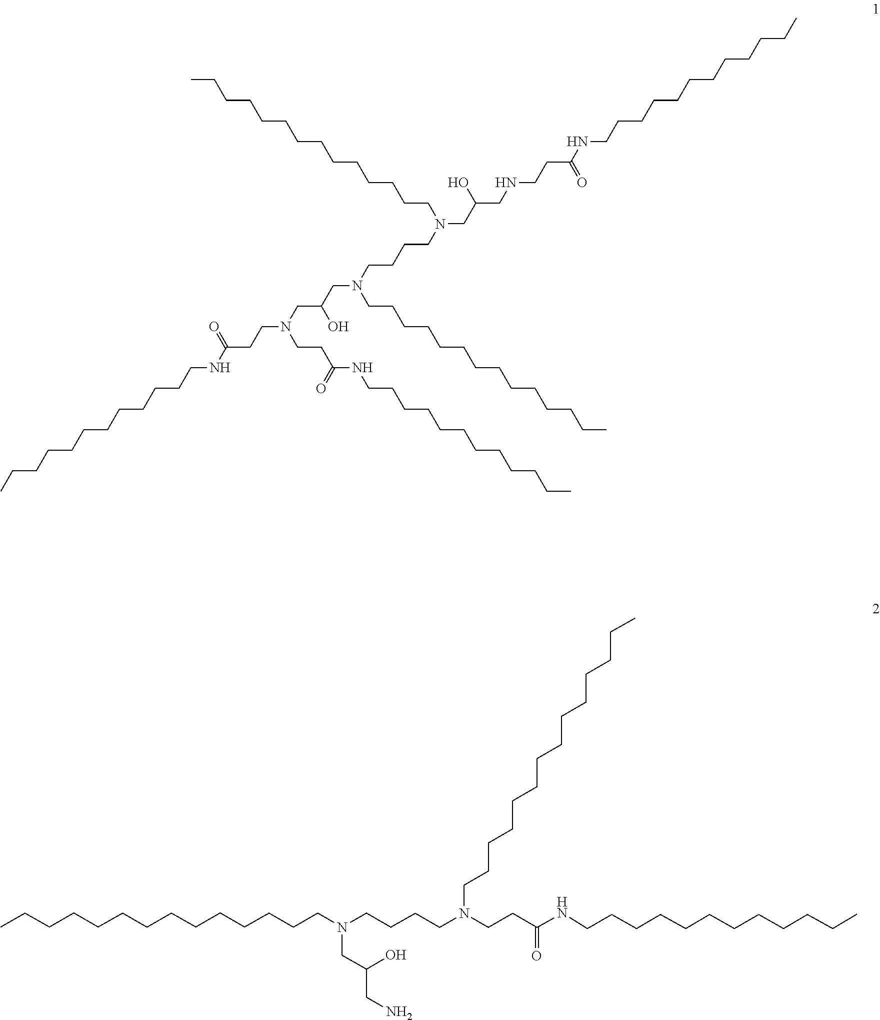

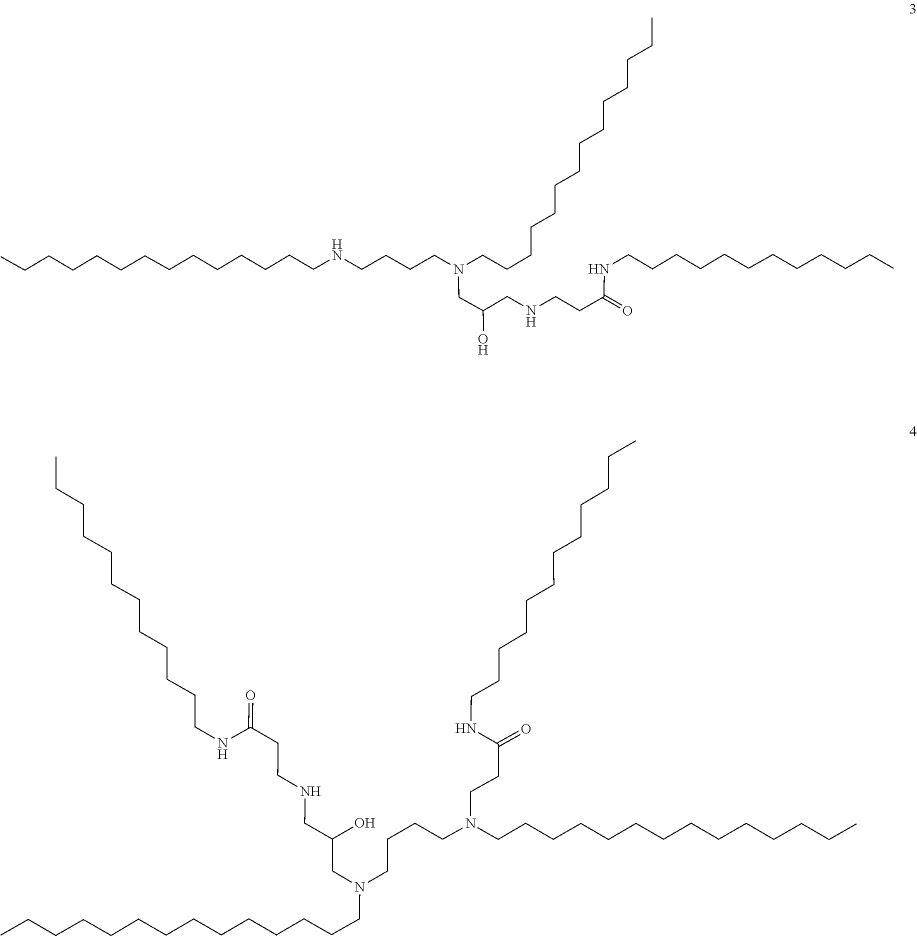

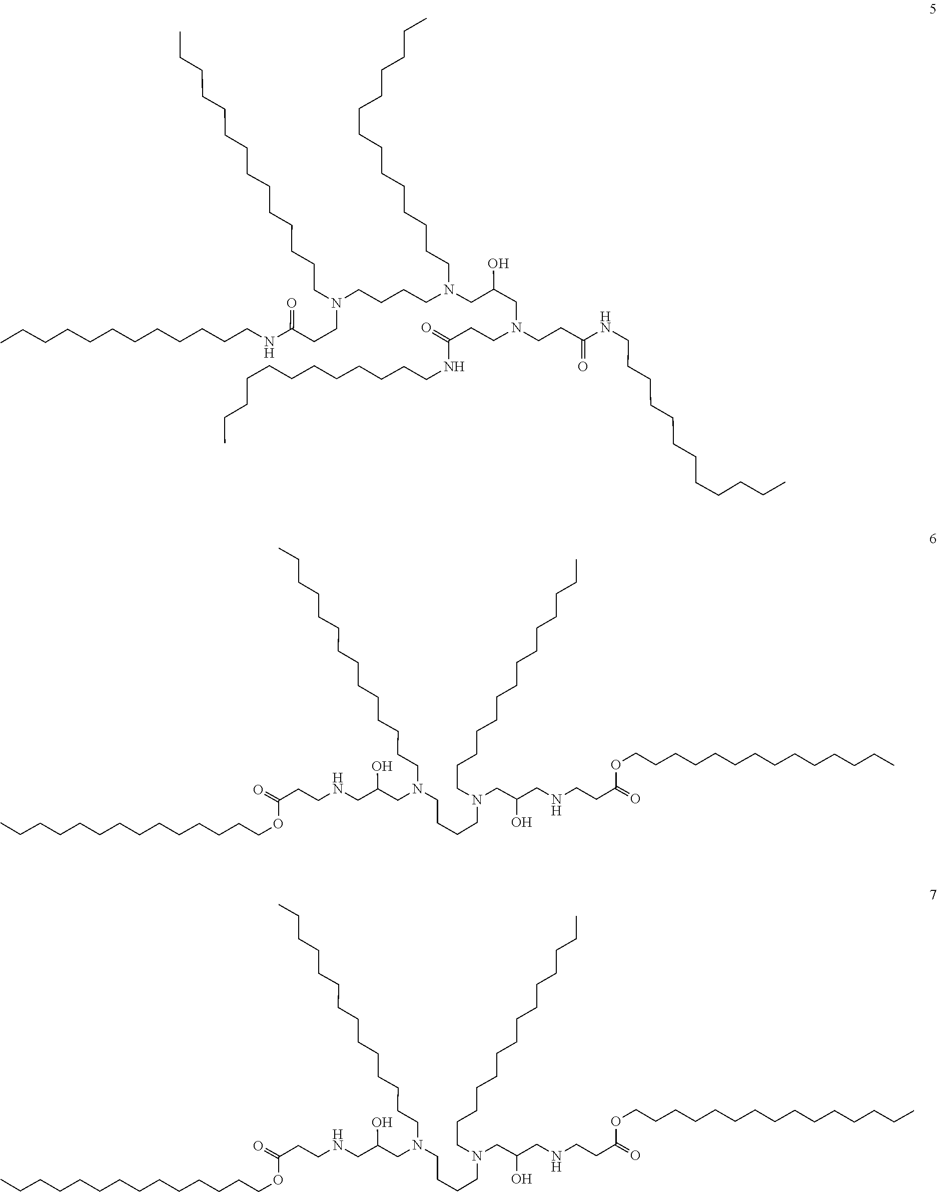

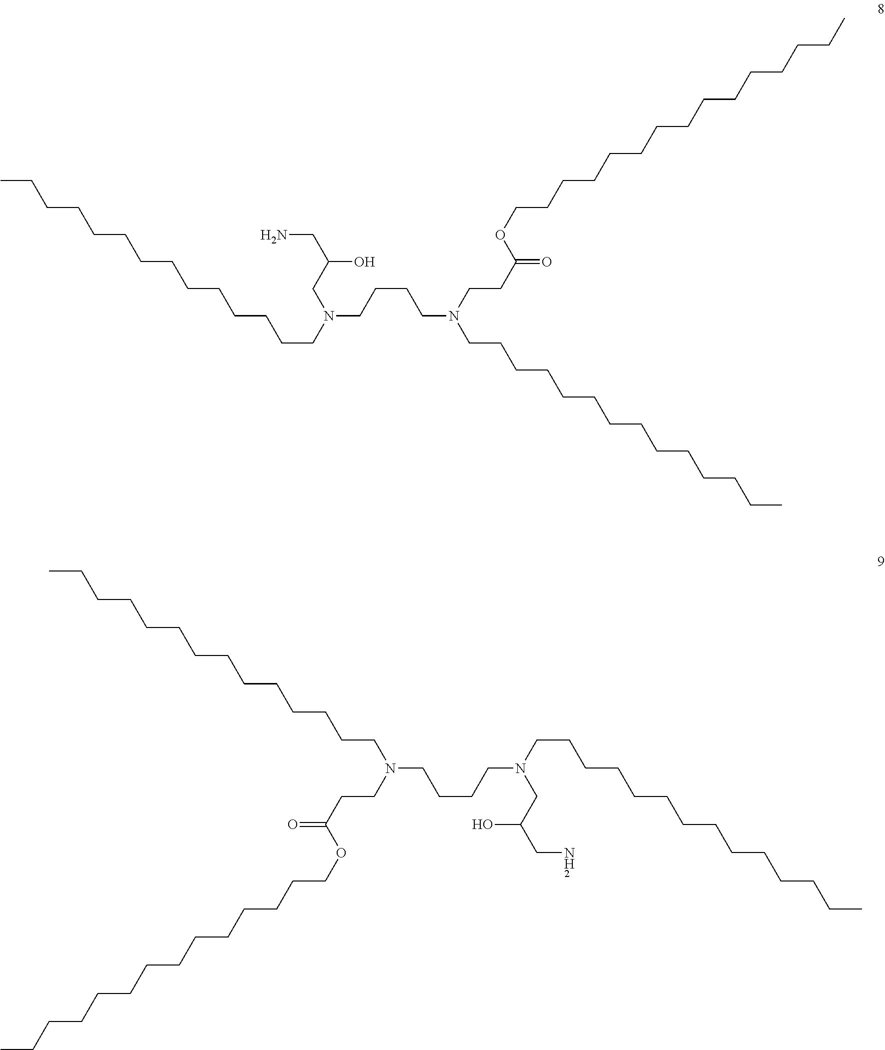

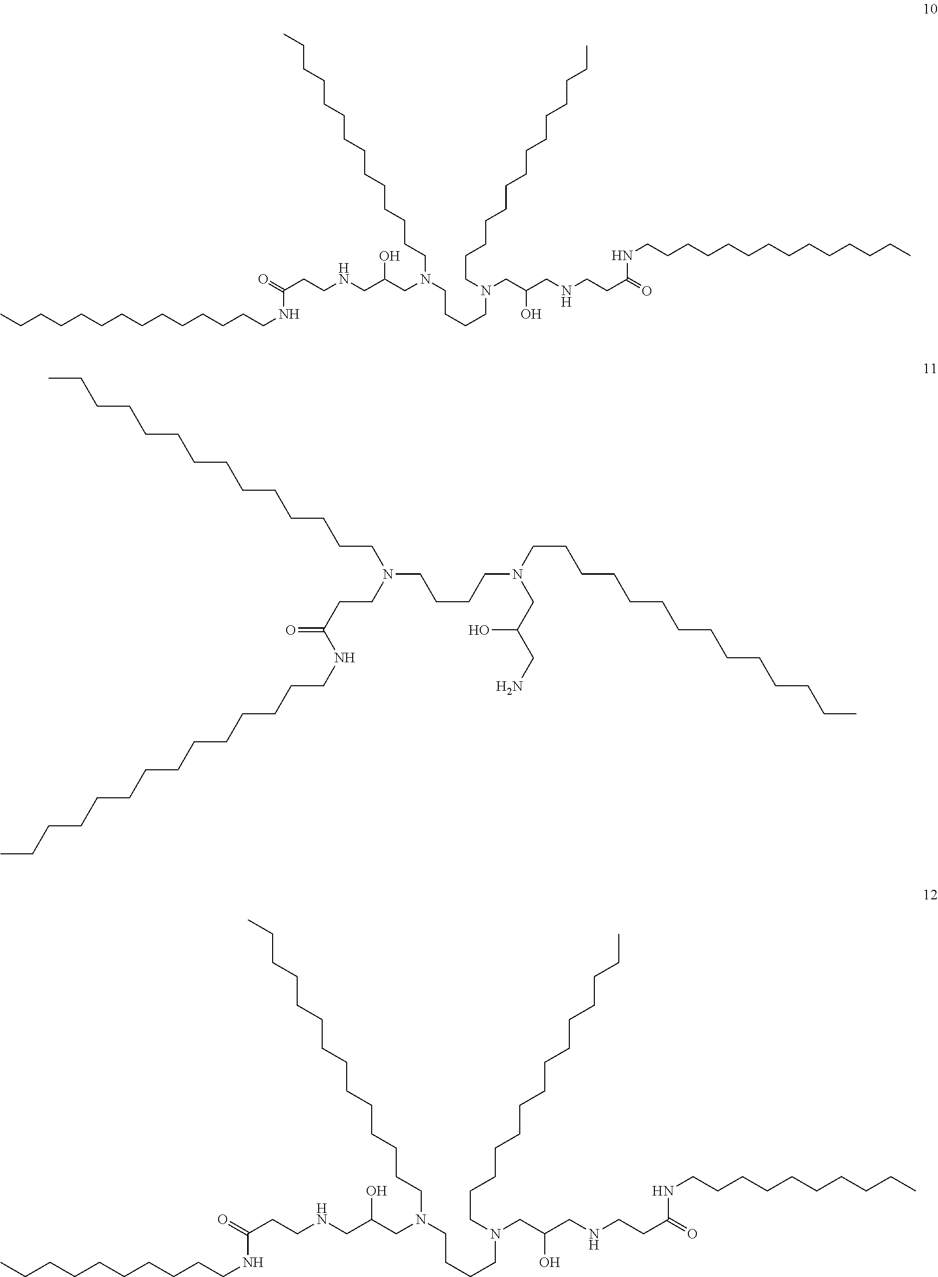

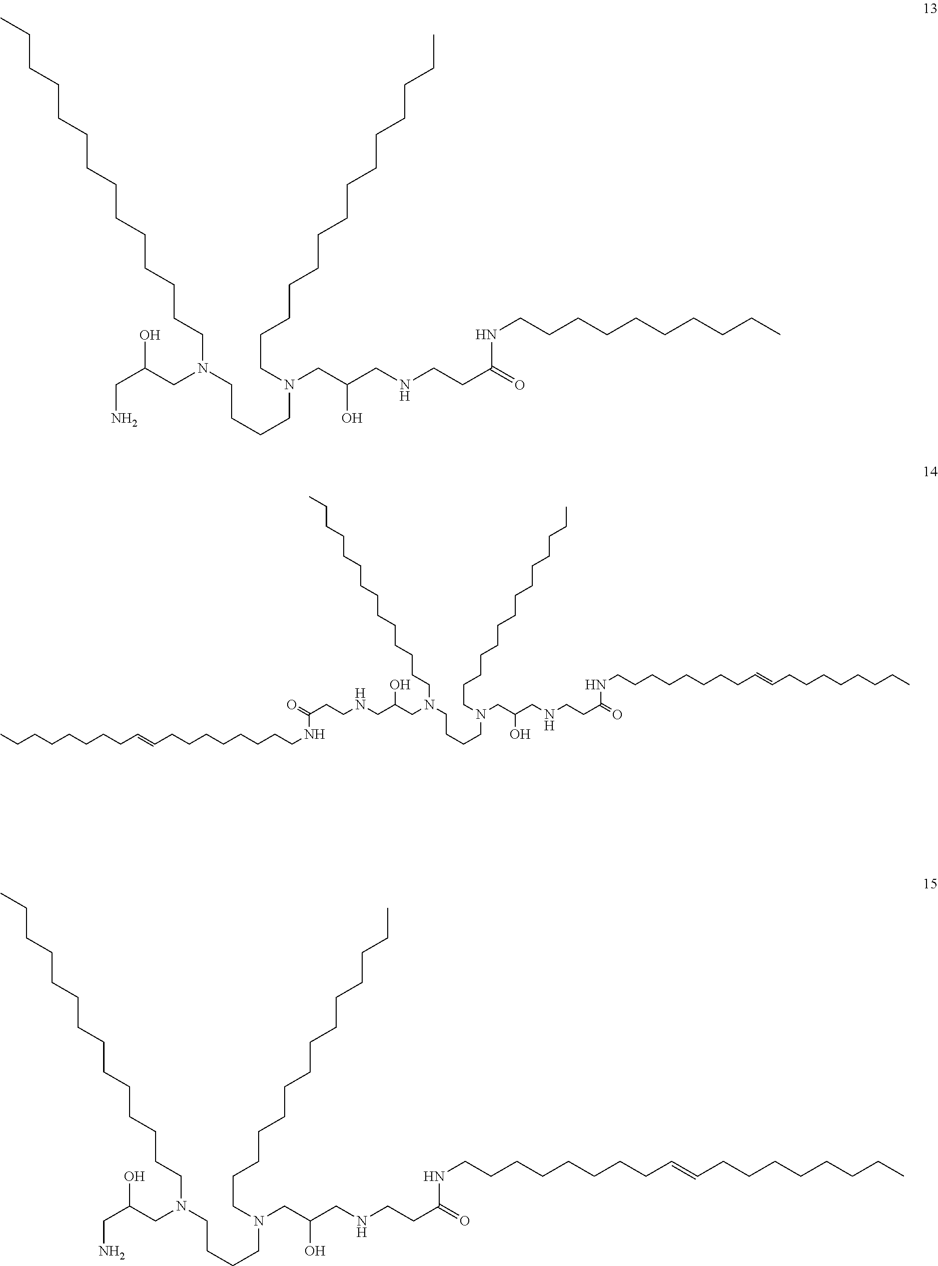

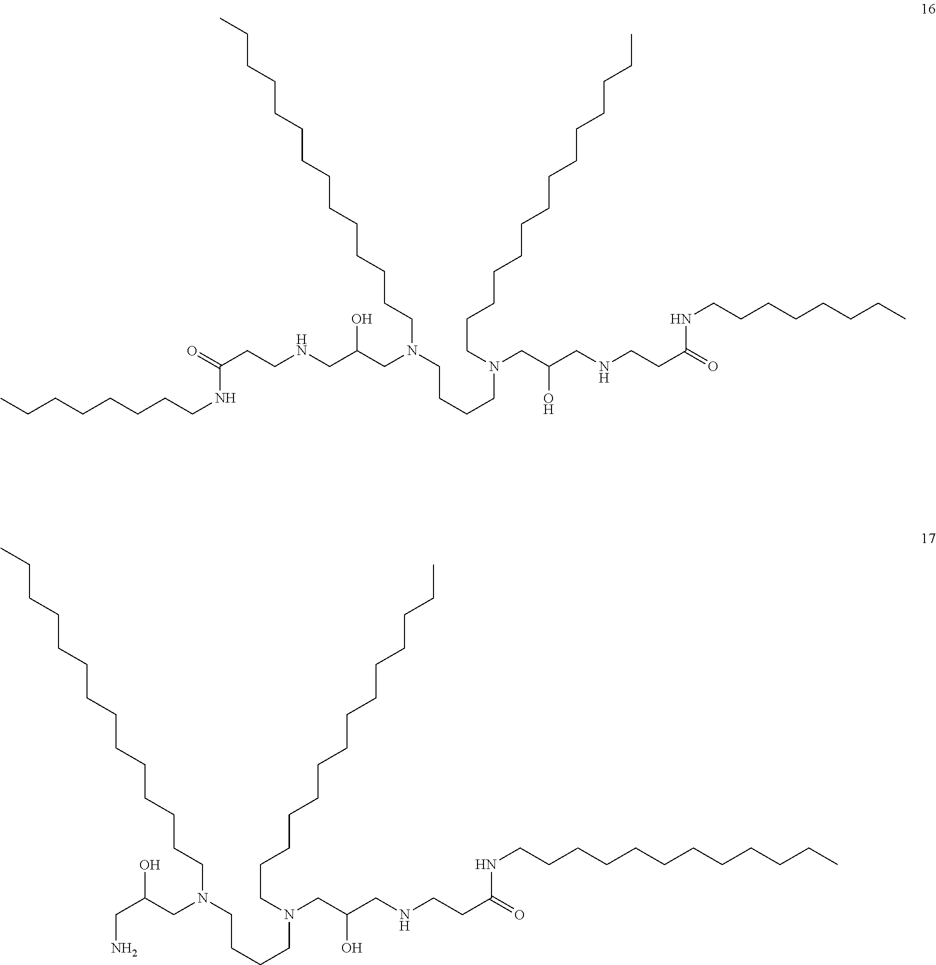

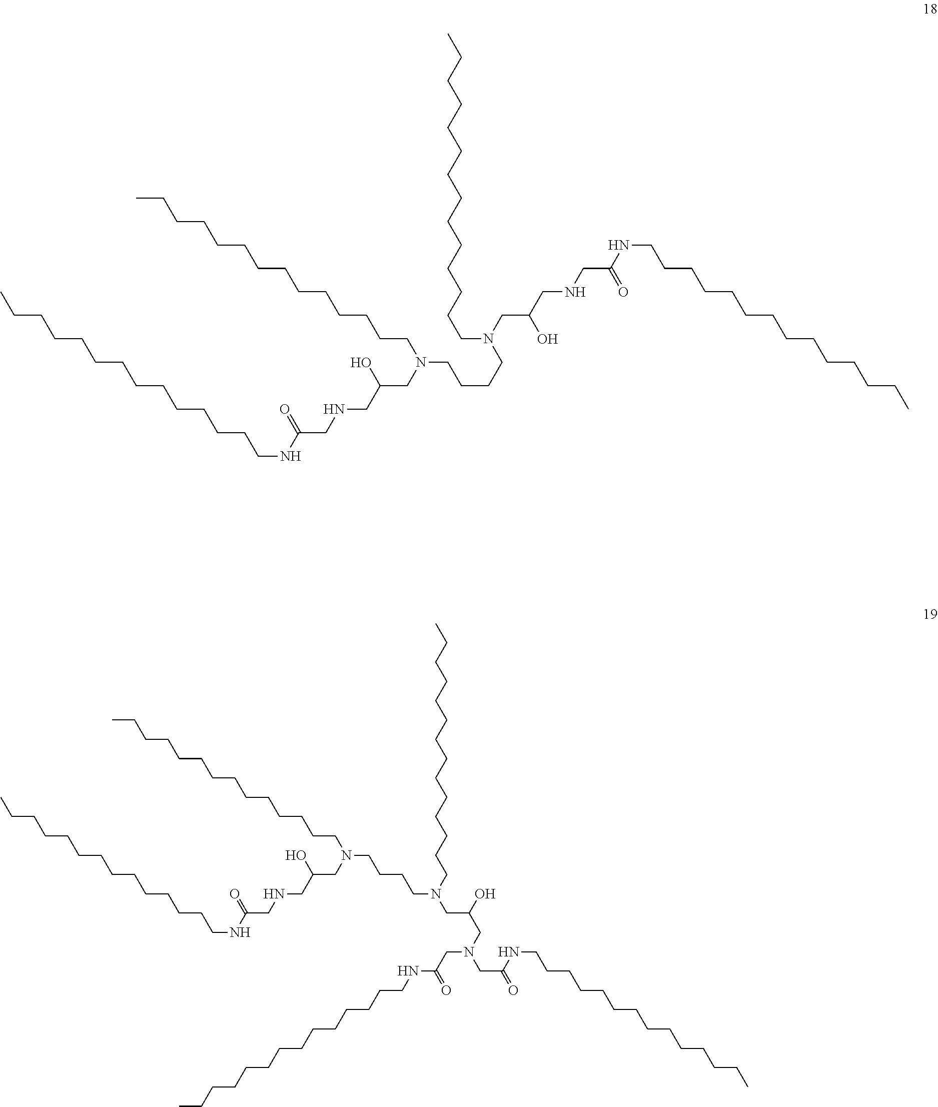

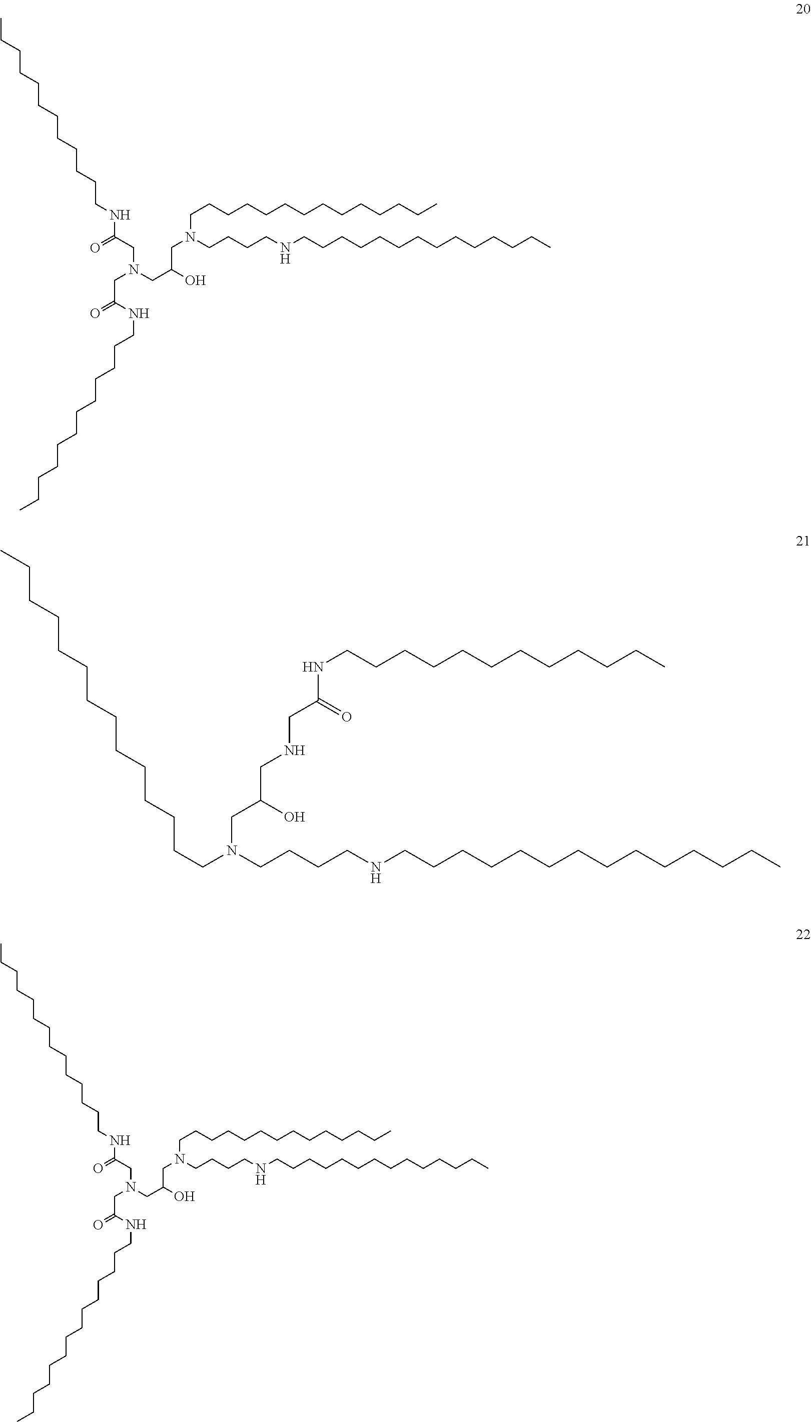

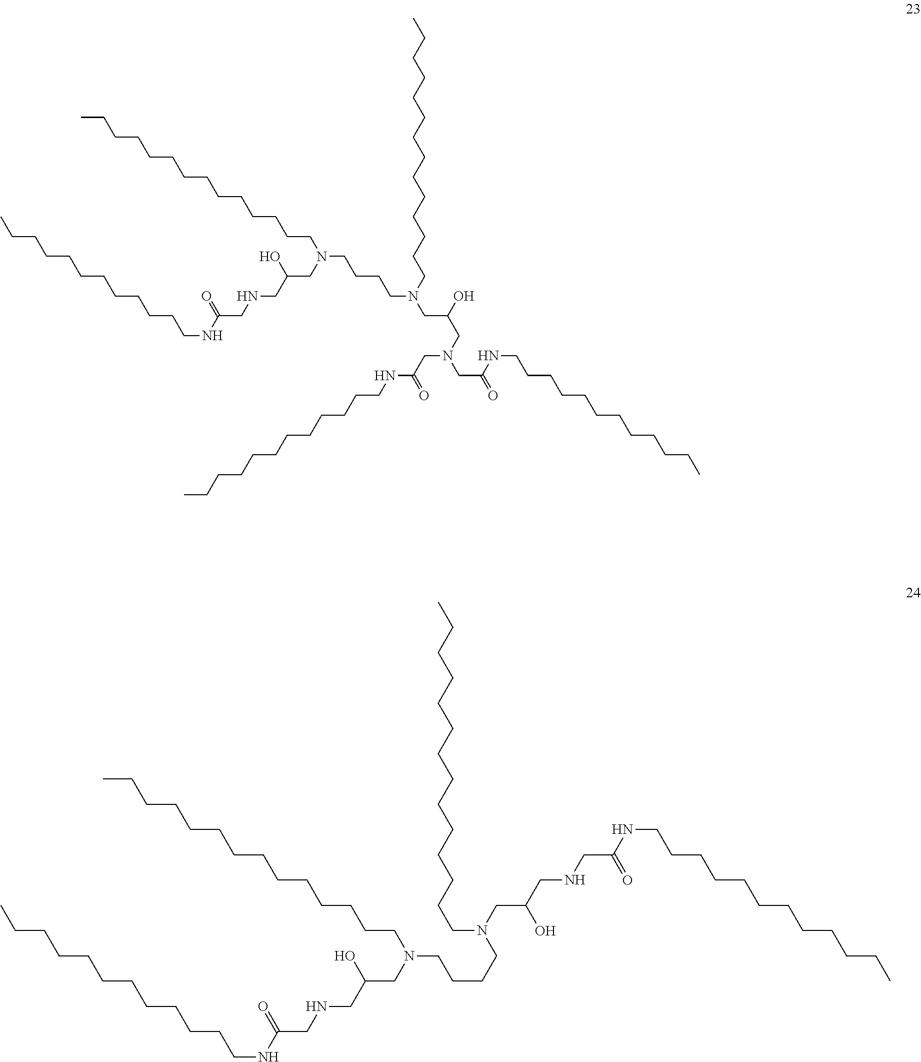

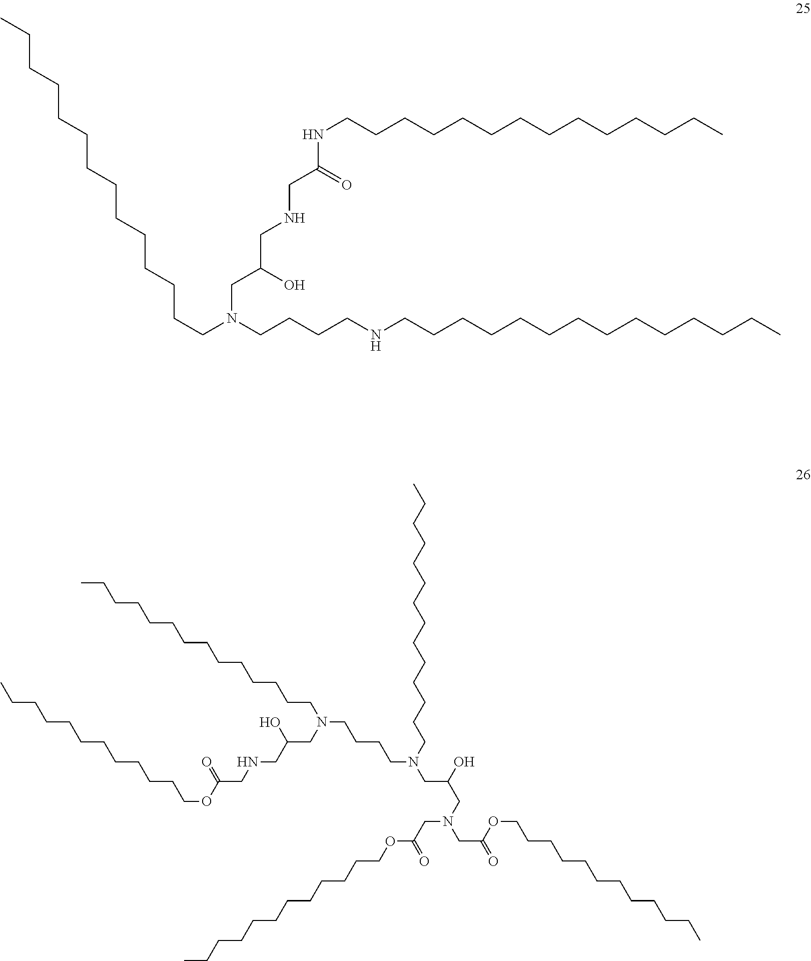

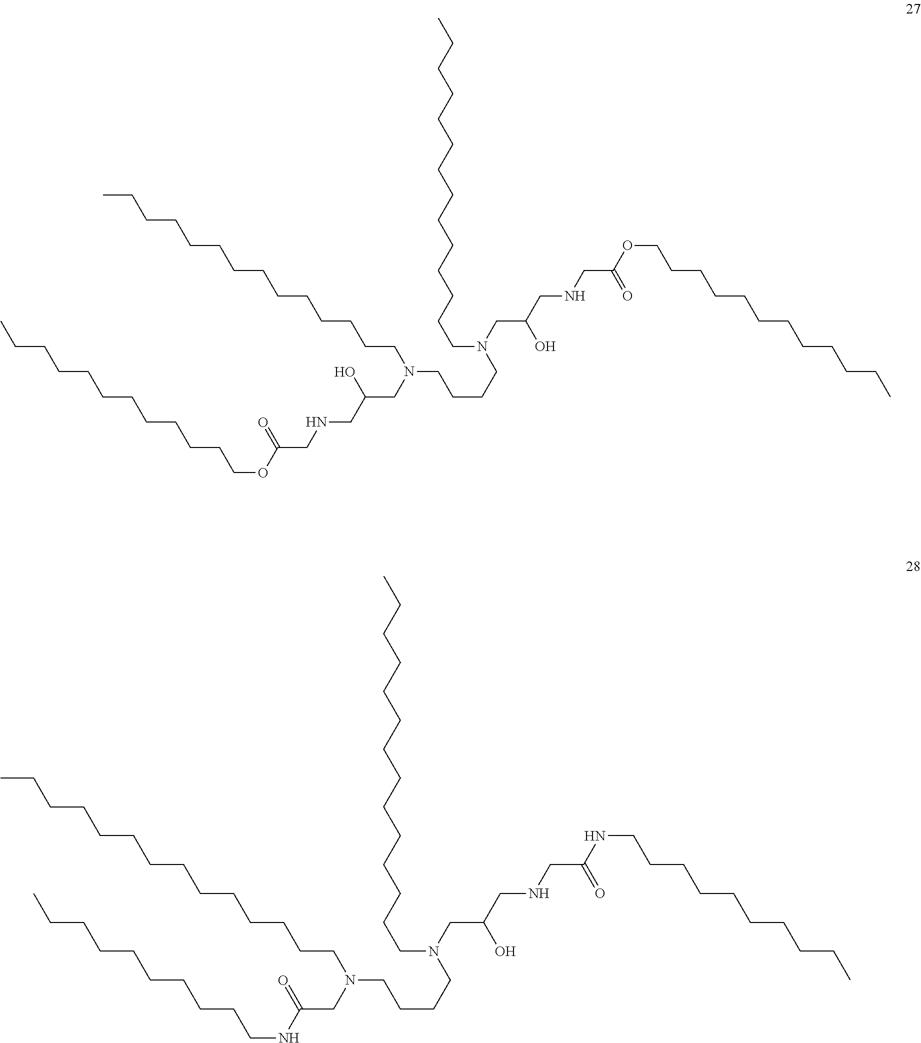

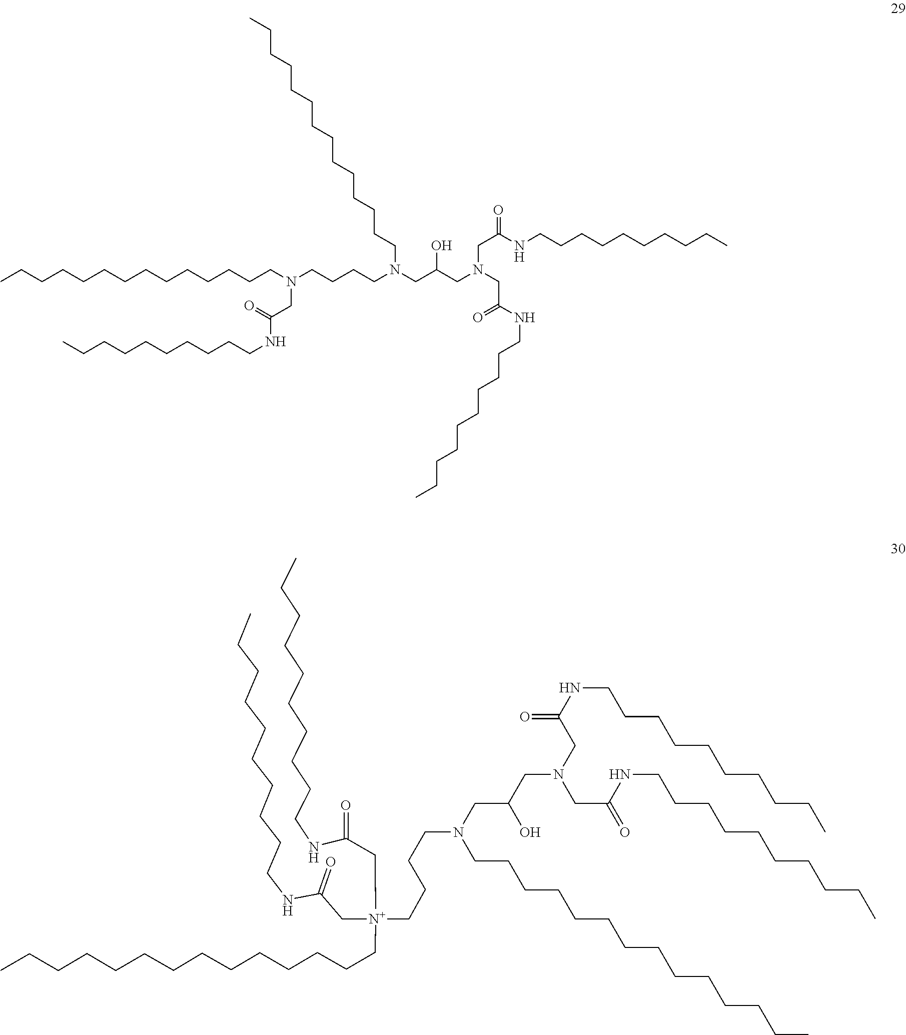

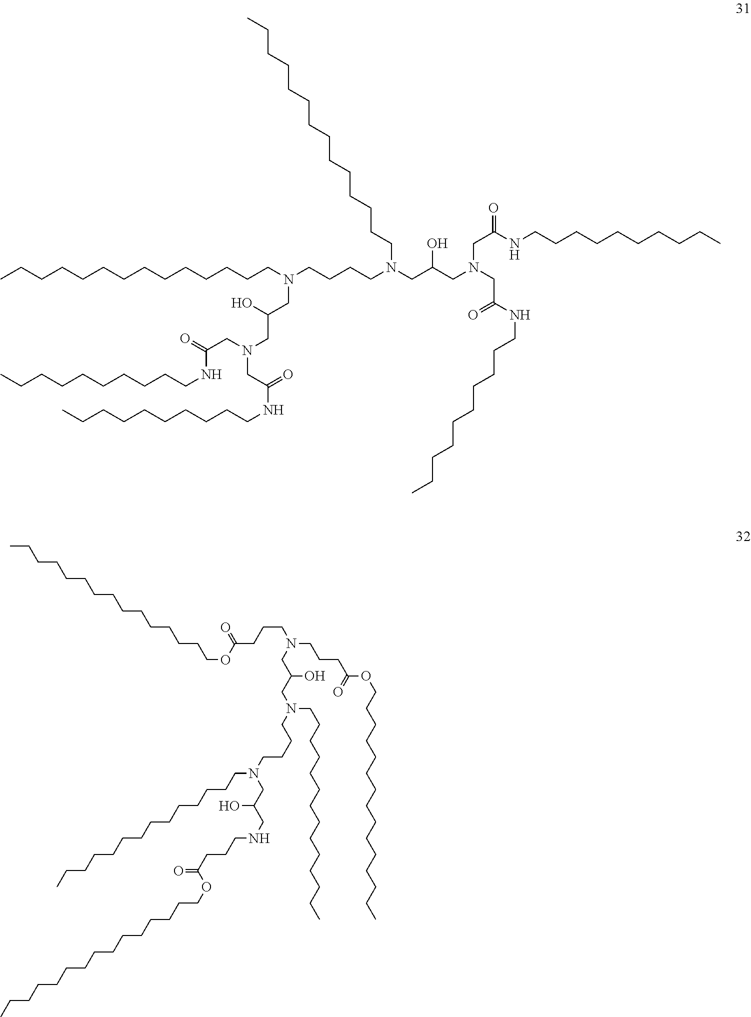

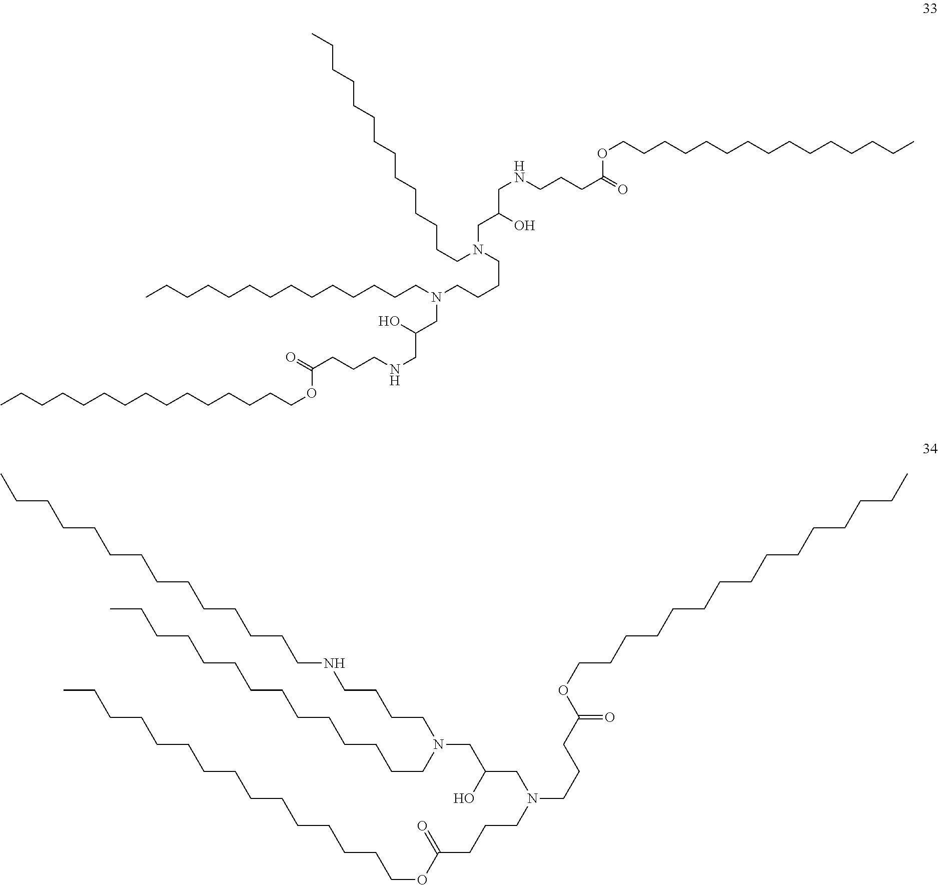

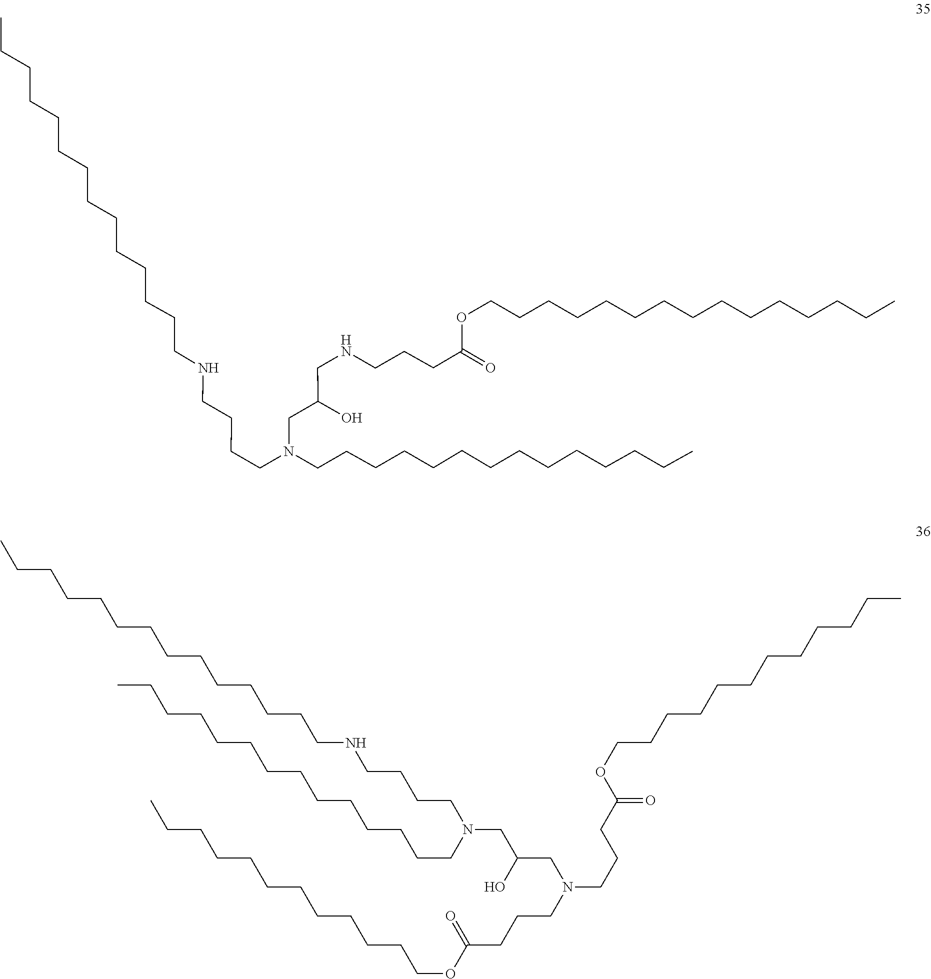

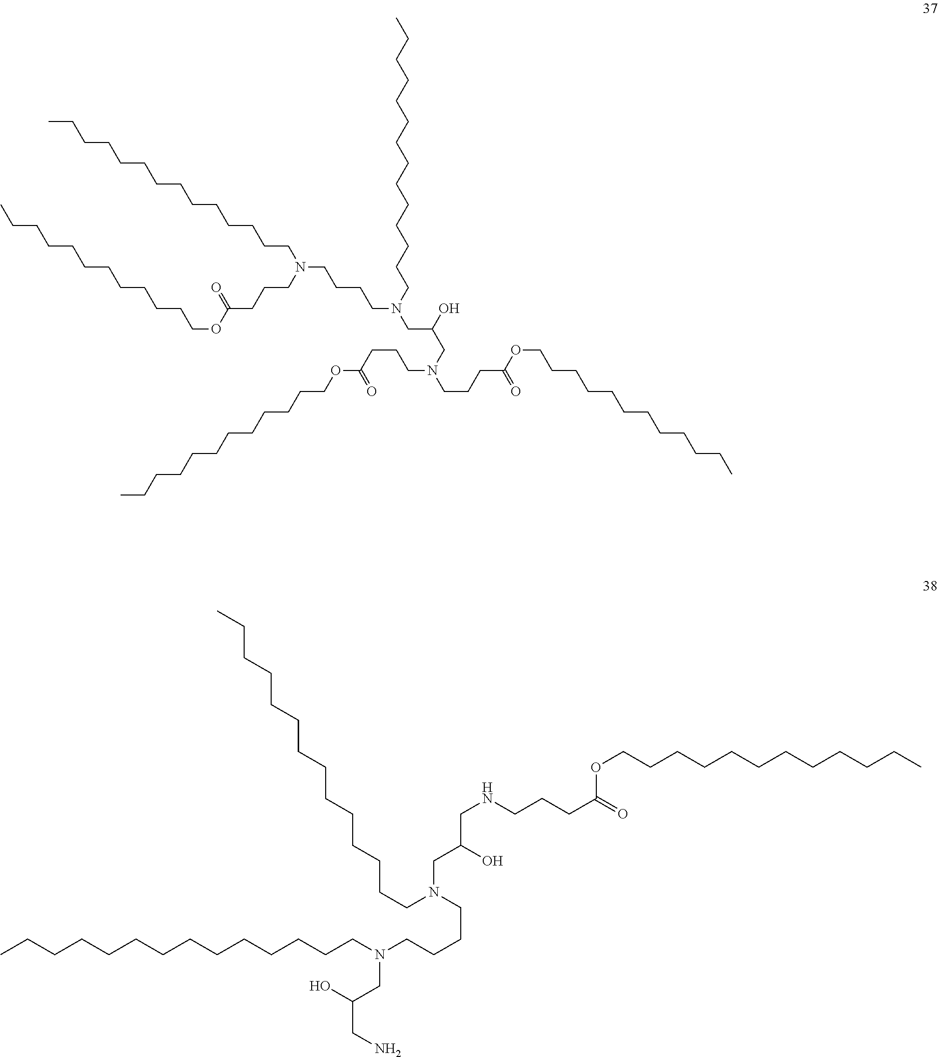

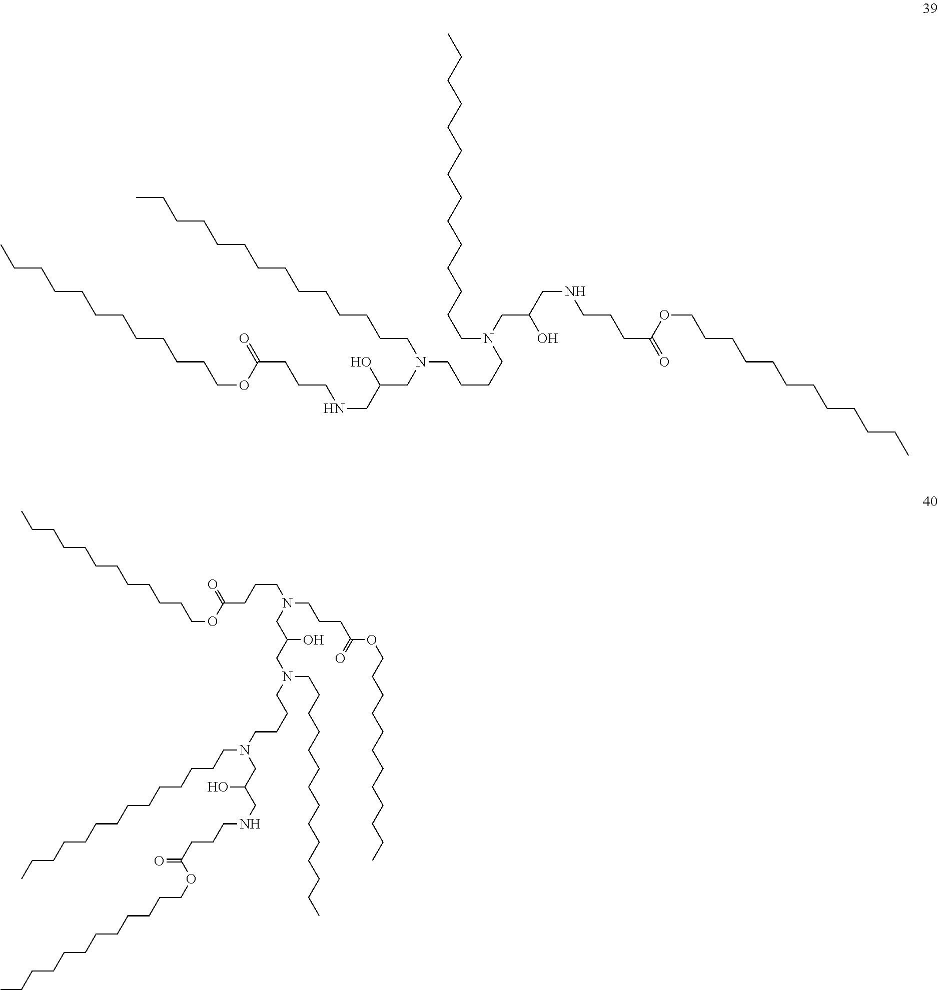

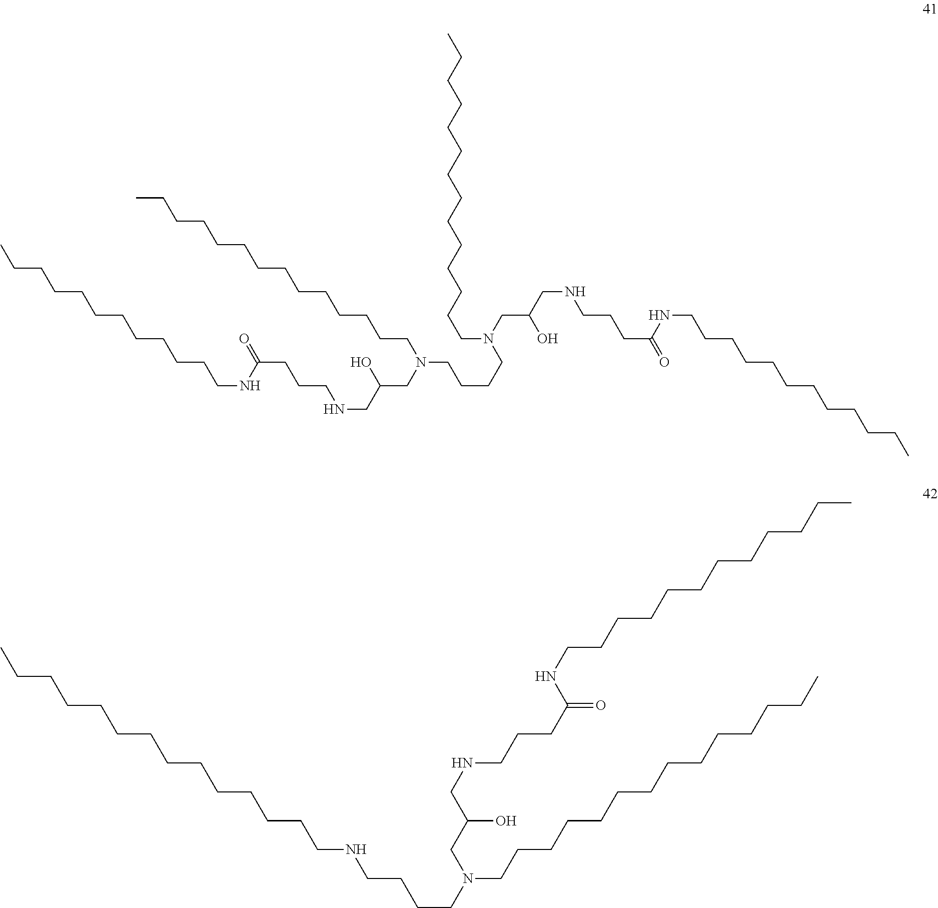

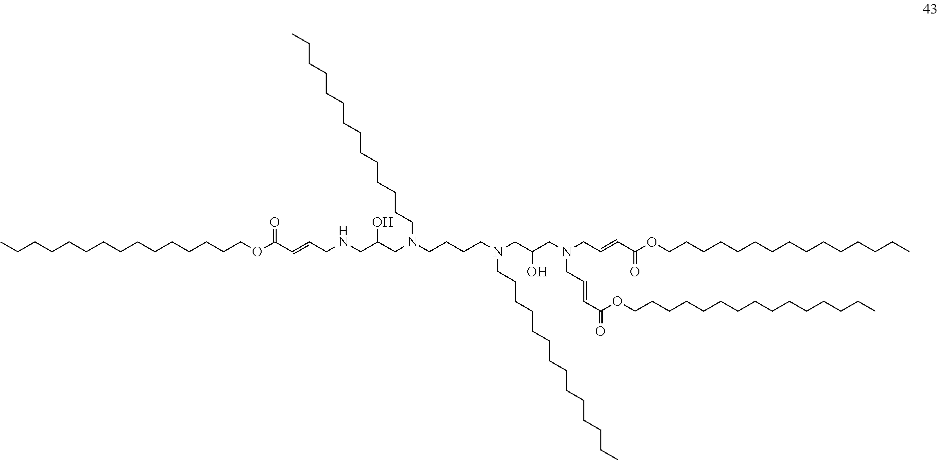 [SMILES: CCCCCCCCCCCCCCCOC(=O)/C=C/CNCC(O)CN(CCCCCCCCCCCCCC)CCCCN(CCCCCCCCCCCCCC)CC(O)CN(C/C=C/C(=O)OCCCCCCCCCCCCCCC)C/C=C/C(=O)OCCCCCCCCCCCCCCC]

The above-described compounds may be synthesized and/or purified by methods described in U.S. Pat. No. 9,901,642 (herein incorporated by reference in its entirety), as well as other methods known in the art. For example, the above-described compounds may be synthesized by reacting an amino component with an unsaturated component, e.g., by the addition of the primary amino group of the amino component to a double bond of the unsaturated component where the double is conjugated with an electrophilic group such as, e.g., carbonyl. In general, the synthetic method includes reacting one equivalent of the amino component with one or more equivalents of the unsaturated component. The amino component comprises a primary amine NH₂—R₃, a diamine, a polyamine or a combination thereof. The unsaturated component comprises of at least one first intermediate having the structure R₁—X₁—Y—(CR₄R₅)ₙ—Br and the second intermediate having the structure R₂—X₂—Z—(CR₆R₇)ₘ—Br, wherein in (CR₄R₅)ₙ and (CR₆R₇)ₘ portions of the structures, each R₄ is the same or different, each R₅ is the same or different, each R₆ is the same or different, and each R₇ is the same or different, wherein the first and the second intermediates are the same or different. In some instances, the first and/or the second intermediate(s) of the unsaturated component can be an acrylate or acrylamide. In certain instances, all the amino groups of the amine NH₂—R₃, a diamine or a polyamine are fully reacted with the unsaturated component to form tertiary amines. In other instances, not all the amino groups are so reacted to form tertiary amines thereby resulting in primary or secondary amines in the lipid molecule.

In some embodiments, the ionizable lipids provided herein include at least one biodegradable group or linkage, such as an ester, amide, anhydride, carbonate, and/or orthoester linkage.

Ionizable lipids described herein refer to lipids that have at least one protonatable or deprotonatable group, such that the lipid is positively charged at a pH at or below physiological pH (e.g., pH 7.4) and neutral at a second pH, preferably at or above physiological pH. Preferably, the ionizable lipids provided herein have a pKa of the protonatable group in the range of about 4 to about 11, e.g., about 4 to about 8, about 4.5 to about 7.5, about 4.5 to about 6, about 5 to about 7.5, such as between about 5.5 and 6.9, between about 6 and about 7.5, and between about 6.5 and about 7.5, when incorporated into the lipoplex compositions, for example, liposomes, lipid nanoparticles or other lipid complexes.

The central amine moieties of an ionizable lipid according to structure I, structure II or the other N-containing lipid structures depicted herein may be protonated at a physiological pH. Thus, the ionizable lipid may have a positive or partial positive charge at physiological pH.

The skilled artisan will recognize that, although some of the ionizable lipid molecules are shown here for convenience in their neutral (unprotonated) forms, these molecules will exist in a partially or fully protonated form in solutions of appropriate pH, and that the present invention encompasses the molecules in all their protonated, unprotonated, ionized and non-ionized forms without limitation, unless specifically indicated otherwise.

In some embodiments, the lipoplex comprises at least one ionizable lipid having structure I and one or more helper lipids. In some embodiments, the one or more helper lipids may be selected from other ionizable lipids, cationic lipids, neutral lipids, phospholipids, sterols, sterol derivatives, or combinations thereof.

In some embodiments, additional ionizable lipids described in U.S. Pat. Nos. 7,173,154, 8,034,977, 9,856,496, and U.S. Publication No. US 2019/0060482 are contemplated for use in the present compositions and methods (wherein the reference is incorporated by reference in their entireties).

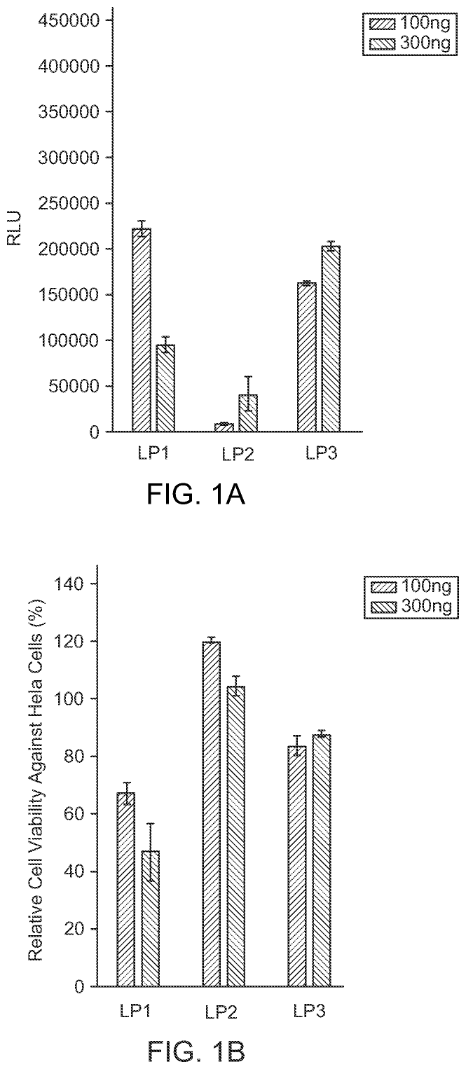

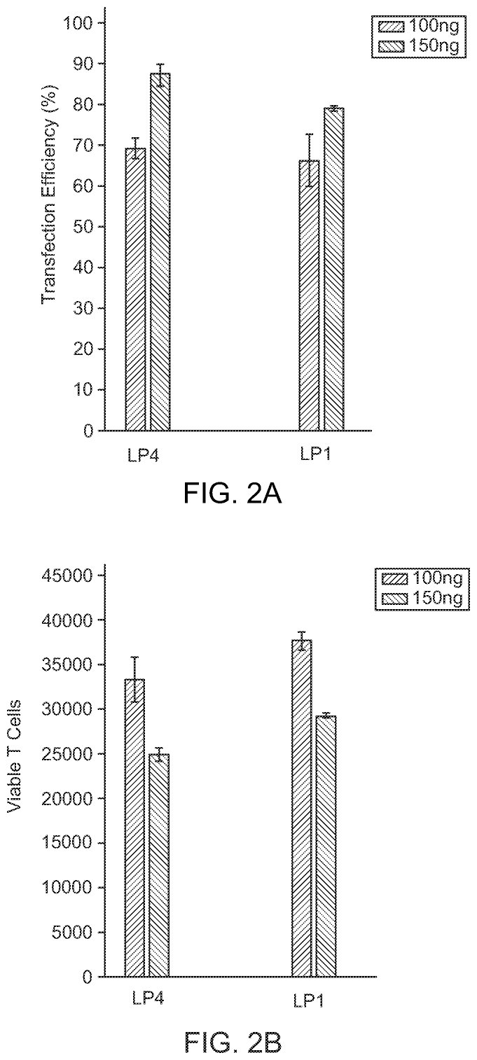

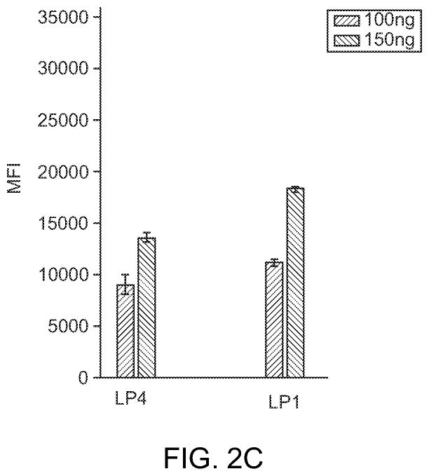

In some embodiments, additional ionizable lipids contemplated for use in the present lipoplex compositions and methods are provided below (compounds X-1 to X-16) and any others from FIGS. 1 and 2 of Han et al (2021); “An ionizable lipid toolbox for RNA delivery; vol. 12, page 7233; incorporated by reference in its entirety.

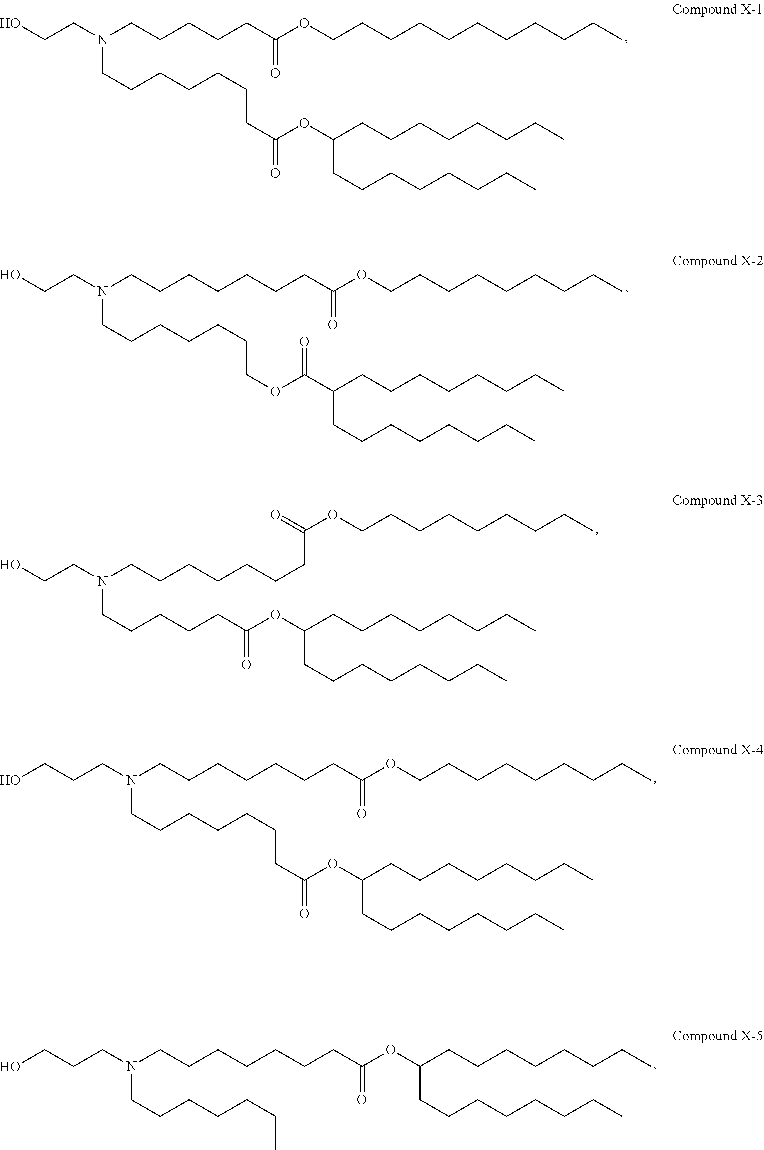 [SMILES: CCCCCCCCCCCOC(=O)CCCCCN(CCO)CCCCCCCC(=O)OC(CCCCCCCC)CCCCCCCC ; CCCCCCCCCOC(=O)CCCCCCCN(CCO)CCCCCCCOC(=O)C(CCCCCCCC)CCCCCCCC ; CCCCCCCCCOC(=O)CCCCCCCN(CCO)CCCCCC(=O)OC(CCCCCCCC)CCCCCCCC ; CCCCCCCCCOC(=O)CCCCCCCN(CCCO)CCCCCCCC(=O)OC(CCCCCCCC)CCCCCCCC ; CCCCCCCCC(CCCCCCCC)OC(=O)CCCCCCCN(CCCO)CCCCCCC]

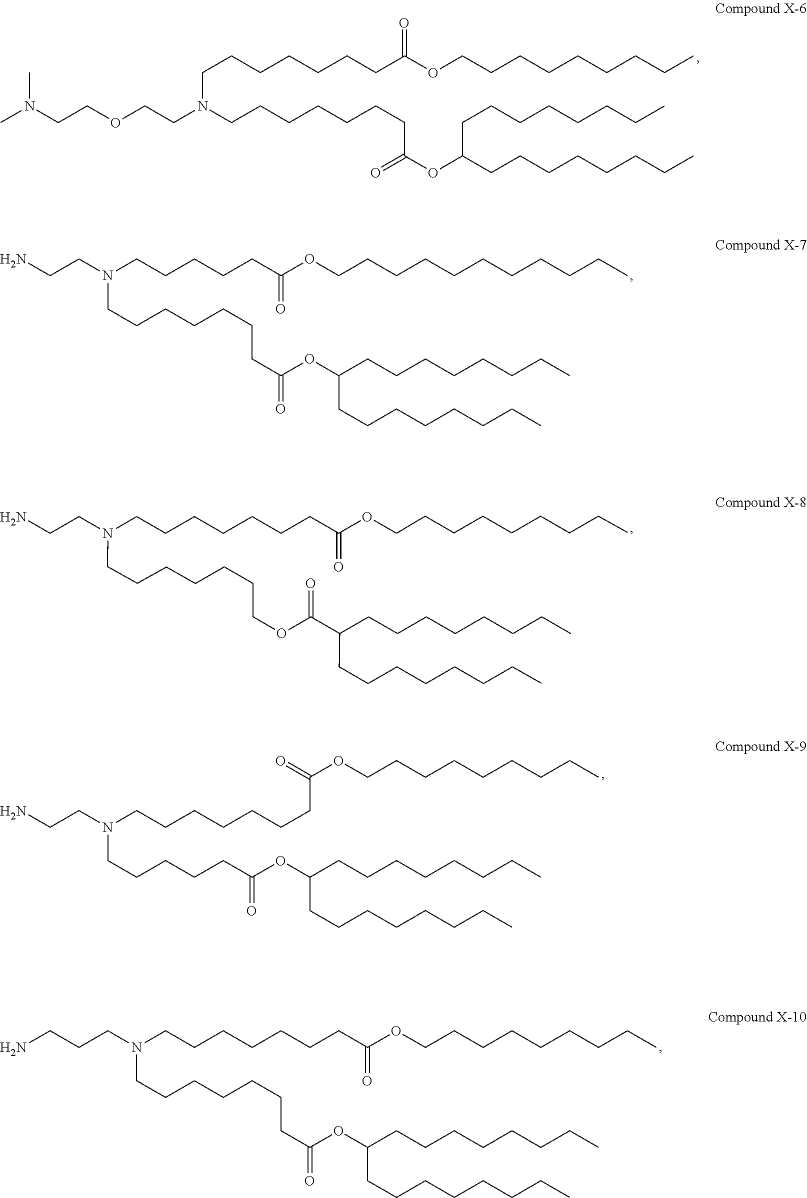 [SMILES: CCCCCCCCCOC(=O)CCCCCCCN(CCCCCCCC(=O)OC(CCCCCCCC)CCCCCCCC)CCOCCN(C)C ; CCCCCCCCCCCOC(=O)CCCCCN(CCN)CCCCCCCC(=O)OC(CCCCCCCC)CCCCCCCC ; CCCCCCCCCOC(=O)CCCCCCCN(CCN)CCCCCCCOC(=O)C(CCCCCCCC)CCCCCCCC ; CCCCCCCCCOC(=O)CCCCCCCN(CCN)CCCCCC(=O)OC(CCCCCCCC)CCCCCCCC ; CCCCCCCCCOC(=O)CCCCCCCN(CCCN)CCCCCCCC(=O)OC(CCCCCCCC)CCCCCCCC]

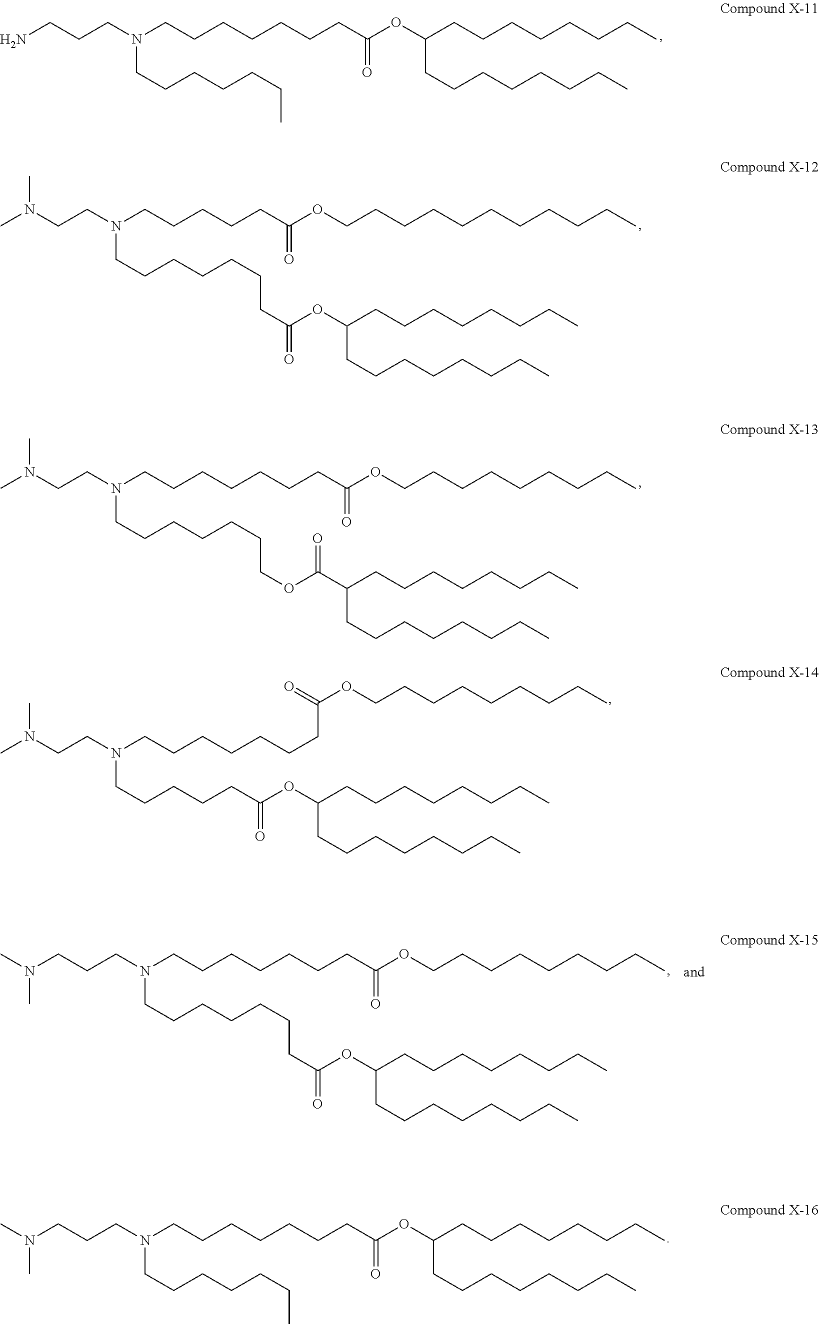

In some embodiments, at least one helper lipid may be selected from, for example, the group consisting of DOTMA, DOTAP, DMRIE, DC-Chol, DDAB, DOSPA, DOSPER, DOGS, TMTPS, TMTOS, TMTLS, TMTMS, TMDOS, N-1-dimethyl-N-1-(2,3-diaoleoyloxypropyl)-2-hydroxypropane-1,3-diamine, N-1-dimethyl-N-1-(2,3-diamyristyloxypropyl)-2-hydroxypropane-1,3-diamine, N-1-dimethyl-N-1-(2,3-diapalmityloxypropyl)-2-hydroxypropane-1,3-diamine, N-1-dimethyl-N-1-(2,3-diaoleoyloxypropyl)-2-(3-amino-2-hydroxypropyloxy)propane-1,3-diamine, N-1-dimethyl-N-1-(2,3-diamyristyloxypropyl)-2-(3-amino-2-hydroxypropyloxy)propane-1,3-diamine, N-1-dimethyl-N-1-(2,3-diapalmityloxypropyl)-2-(3-amino-2-hydroxypropyloxy)propane-1,3-diamine, L-spermine-5-carboxyl-3-(DL-1,2-dipalmitoyl-dimethylaminopropyl-β-hydroxyethylamine, 3,5-(N,N-di-lysyl)-diaminobenzoyl-glycyl-3-(DL-1,2-dipalmitoyl-dimethylaminopropyl-β-hydroxyethylamine), L-Lysine-bis(O,O′-oleoyl-β-hydroxyethyl)amide dihydrochloride, L-Lysine-bis-(O,O′-palmitoyl-β-hydroxyethyl)amide dihydrochloride, 1,4-bis[(3-(3-aminopropyl)-alkylamino)-2-hydroxypropyl)piperazine, L-Lysine-bis-(O,O′-myristoyl-β-hydroxyethyl)amide dihydrochloride, L-Ornithine-bis-(O,O′-myristoyl-β-hydroxyethyl)amide dihydrochloride, L-Ornithine-bis-(O,O′-oleoyl-β-hydroxyethyl)amide dihydrochloride, 1,4-bis[(3-(3-aminopropyl)-oleylamino)-2-hydroxypropyl]piperazine, L-Ornithine-bis-(O,O′-palmitoyl-β-hydroxyethyl)amide dihydrochloride, 1,4,-bis[(3-amino-2-hydroxypropyl)-oleylamino]-butane-2,3-diol, 1,4,-bis[(3-amino-2-hydroxypropyl)-palmitylamino]-butane-2,3-diol, 1,4,-bis[(3-amino-2-hydroxypropyl)-myristylamino]-butane-2,3-diol, 1,4-bis[(3-oleylamino)propyl]piperazine, L-Arginine-bis-(O,O′-oleoyl-β-hydroxyethyl)amide dihydrochloride, bis[(3-(3-aminopropyl)-myristylamino)2-hydroxypropyl]piperazine, L-Arginine-bis-(O,O′-palmitoyl-β-hydroxyethyl)amide dihydrochloride, L-Serine-bis-(O,O′-oleoyl-β-hydroxyethyl)amide dihydrochloride, 1,4-bis[(3-(3-aminopropyl)-palmitylamino)-2-hydroxypropyl]piperazine, Glycine-bis-(O,O′-palmitoyl-β-hydroxyethyl)amide dihydrochloride, Sarcosine-bis-(O,O′-palmitoyl-β-hydroxyethyl)amide dihydrochloride, L-Histidine-bis-(O,O′-palmitoyl-β-hydroxyethyl)amide dihydrochloride, cholesteryl-3β-carboxyl-amidoethylenetrimethylammonium iodide, 1,4-bis[(3-myristylamino)propyl]piperazine, 1-dimethylamino-3-trimethylammonio-DL-2-propyl-cholesteryl carboxylate iodide, cholesteryl-3β-carboxyamidoethyleneamine, cholesteryl-3β-oxysuccinamidoethylenetrimethylammonium iodide, 1-dimethylamino-3-trimethylammonio-DL-2-propyl-cholesteryl-3β-oxysuccinate iodide, 2-[(2-trimethylammonio)-ethylmethylamino] ethyl-cholesteryl-3β-oxysuccinate iodide, 3β[N—(N′,N′-dimethylaminoethane)carbamoyl]cholesterol, and 3p-[N-(polyethyleneimine)-carbamoyl] cholesterol,1,4-bis[(3-palmitylamino)propyl]piperazine, L-Ornithylglycyl-N-(1-heptadecyloctadecyl)glycinamide, N²,N⁵-Bis(3-aminopropyl)-L-ornithylglycyl-N-(1-heptadecyloctadecyl)glycinamide, 1,4-bis[(3-(3-amino-2-hydroxypropyl)-alkylamino)-2-hydroxypropyl]piperazine N²—[N²,N⁵-Bis(3-aminopropyl)-L-ornithyl]-N,N-dioctadecyl-L-glutamine,N²—[N²,N⁵-Bis(aminopropyl)-L-ornithyl]-N—N-dioctadecyl-L-α-glutamine, 1,4-bis[(3-(3-amino-2-hydroxypropyl)-oleylamino)2-hydroxypropyl]piperazine, N²—[N²,N⁵-Bis(aminopropyl)-L-ornithyl]-N—N-dioctadecyl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dioctadecyl-L-glutaminyl]-L-glutamic acid, N²—[N²,N⁵-Bis(3-aminopropyl)-L-ornithyl]-N,N-diolyl-L-glutamine, N²—[N²,N⁵-Bis(aminopropyl)-L-ornithyl]-N—N-dioleyl-L-α-glutamine,4-bis[(3-(3-amino-2-hydoxypropyl)-myristylamino)-2-hydroxypropyl]piperazine, N²—[N²,N⁵-Bis(aminopropyl)-L-ornithyl]-N—N-dioleyl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dioleyl-L-glutaminyl]-L-glutamic acid, 1,4-bis[(3-(3-aminopropyl)-oleylamino)propyl]piperazine, N²—[N²,N⁵-Bis(3-aminopropyl)-L-ornithyl]-N,N-dipalmityl-L-glutamine,N²—[N²,N⁵-Bis(aminopropyl)-L-ornithyl]-N—N-dipalmityl-L-α-glutamine, N²—[N²,N⁵-Bis(aminopropyl)-L-ornithyl]-N—N-dipalmityl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dipalmityl-L-glutaminyl]-L-glutamic acid, N²—[N²,N⁵-Bis(3-aminopropyl)-L-ornithyl]-N,N-dimyristyl-L-glutamine, N²—[N²,N⁵-Bis(aminopropyl)-L-ornithyl]-N—N-dimyristyl-L-α-glutamine, N²—[N²,N⁵-Bis(aminopropyl)-L-ornithyl]-N—N-dimyristyl-L-α-asparagine, 1,4-bis[(3-(3-amino-2-hydroxypropyl)-palmitylamino)-2-hydroxypropyl]piperazine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dimyristyl-L-glutaminyl]-L-glutamic acid, 1,4-bis[(3-(3-aminopropyl)-myristylamino)propyl]piperazine, N²—[N²,N⁵-Bis(3-aminopropyl)-L-ornithyl]-N,N-dilaureyl-L-glutamine, N²—[N²,N⁵-Bis(aminopropyl)-L-ornithyl]-N—N-dilaureyl-L-α-glutamine, N²—[N²,N⁵-Bis(aminopropyl)-L-ornithyl]-N—N-dilaureyl-L-α-asparagine, N—[N²—[N²,N⁵-Bis[(1,1-dimethylethoxy)carbonyl]-N²,N⁵-bis[3-[(1,1-dimethylethoxy)carbonyl]aminopropyl]-L-ornithyl-N—N-dilaureyl-L-glutaminyl]-L-glutamic acid, 3-[N′,N″-bis(2-tertbutyloxycarbonylaminoethyl)guanidino]-N,N-dioctadec-9-enylpropionamide, 3-[N′,N″-bis(2-tertbutyloxycarbonylaminoethyl)guanidino]-N,N-dipalmitylpropionamide, 3-[N′,N″-bis(2-tertbutyloxycarbonylaminoethyl)guanidino]-N,N-dimyristylpropionamide, 1,4-bis[(3-(3-aminopropyl)-palmitylamino)propyl]piperazine, 1,4-bis[(3-(3-amino-2-hydroxypropyl)-oleylamino)propyl]piperazine, N,N-(2-hydroxy-3-aminopropyl)-N-2-hydroxypropyl-3-N,N-diolylaminopropane, N,N-(2-hydroxy-3-aminopropyl)-N-2-hydroxypropyl-3-N,N-dipalmitylaminopropane, N,N-(2-hydroxy-3-aminopropyl)-N-2-hydroxypropyl-3-N,N-dimyristylaminopropane, 1,4-bis[(3-(3-amino-2-hydoxypropyl)-myristylamino)propyl]piperazine, [(3-aminopropyl)-bis-(2-tetradecyloxyethyl)]methyl ammonium bromide, [(3-aminopropyl)-bis-(2-oleyloxyethyl)]methyl ammonium bromide, [(3-aminopropyl)-bis-(2-palmityloxyethyl)]methyl ammonium bromide, Oleoyl-2-hydroxy-3-N,N-dimethyamino propane, 2-didecanoyl-1-N,N-dimethylaminopropane, palmitoyl-2-hydroxy-3-N,N-dimethyamino propane, 1,2-dipalmitoyl-1-N,N-dimethylaminopropane, myristoyl-2-hydroxy-3-N,N-dimethyamino propane, 1,2-dimyristoyl-1-N,N-dimethylaminopropane, (3-Amino-propyl)->4-(3-amino-propylamino)-4-tetradecylcarbamoyl-butylcarbamic acid cholesteryl ester, (3-Amino-propyl)->4-(3-amino-propylamino-4-carbamoylbutylcarbamic acid cholesteryl ester, (3-Amino-propyl)->4-(3-amino-propylamino)-4-(2-dimethylamino-ethylcarbamoy 1)-butylcarbamic acid cholesteryl ester, Spermine-5-carboxyglycine (N′-stearyl-N′-oleyl) amide tetratrifluoroacetic acid salt, Spermine-5-carboxyglycine (N′-stearyl-N′-elaidyl) amide tetratrifluoroacetic acid salt, Agmatinyl carboxycholesterol acetic acid salt, Spermine-5-carboxy-β-alanine cholesteryl ester tetratrifluoroacetic acid salt, 2,6-Diaminohexanoeyl β-alanine cholesteryl ester bistrifluoroacetic acid salt, 2,4-Diaminobutyroyl β-alanine cholesteryl ester bistrifluoroacetic acid salt, N,N-Bis (3-aminopropyl)-3-aminopropionyl β-alanine cholesteryl ester tristrifluoroacetic acid salt, [N,N-Bis(2-hydroxyethyl)-2-aminoethyl]aminocarboxy cholesteryl ester, Stearyl carnitine ester, Palmityl carnitine ester, Myristyl camitine ester, Stearyl stearoyl carnitine ester chloride salt, L-Stearyl Stearoyl Carnitine Ester, Stearyl oleoyl carnitine ester chloride, Palmityl palmitoyl carnitine ester chloride, Myristyl myristoyl carnitine ester chloride, L-Myristyl myristoyl carnitine ester chloride, 1,4-bis[(3-(3-amino-2-hydroxypropyl)-palmitylamino)propyl]piperazine, N-(3-aminopropyl)-N,N′-bis-(dodecyloxyethyl)-piperazinium bromide, N-(3-aminopropyl)-N,N′-bis-(oleyloxyethyl)-piperazinium bromide, N-(3-aminopropyl)-N,N′-bis-(palmityloxyethyl)-piperazinium bromide, N-(3-aminopropyl)-N,N′-bis-(myristyloxyethyl)-piperazinium bromide, N-(3-aminopropyl)-N′-methyl-N,N′-(bis-2-dodecyloxyethyl)-piperazinium bromide, N-(3-aminopropyl)-N′-methyl-N,N′-(bis-2-oleyloxyethyl)-piperazinium bromide, N-(3-aminopropyl)-N′-methyl-N,N′-(bis-2-palmityloxyethyl)-piperazinium bromide, N-(3-aminopropyl)-N′-methyl-N,N′-(bis-2-myristyloxyethyl)-piperazinium bromide, 1,4-bis[(3-(3-aminopropyl)-oleylamino)-2-hydroxy-propyl]piperazine, 1,4-bis[(3-(3-aminopropyl)-myristylamino)-2-hydroxy-propyl]piperazine, and 1,4-bis[(3-(3-aminopropyl)-palmitylamino)-2-hydroxy-propyl]piperazine, KL22, KL25, 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA or MC3), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), and (2S)-2-({8-[(3)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

In some embodiments, phospholipids useful in the lipoplex compositions disclosed herein include, but are not limited to, dioleoylphosphoethanolamine (DOPE), diphytanolphosphatidylethanolamine (DPhPE), Lyso-PE (1-acyl-2-hydroxy-sn-glycero-3-phosphoethanolamine), Lyso-PC (1-acyl-3-hydroxy-sn-glycero-3-phosphocholine), distearoylphosphatidylcholine (DSPC), dioleoylphosphatidylcholine (DOPC), dipalmitoylphosphatidylcholine (DPPC), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), 1,2-dioleoyl-sn-glycero-3-phophoethanolamine (trans DOPE), 1-stearoyl-2-oleoyl-phosphatidylcholine (SOPC), dilinoleoylphosphocholine (DLPC), dimyristoylphosphocholine (DMPC), diundecanoylphosphocholine (DUPC), dioleoylphosphatidylglycerol (DOPG), dipalmitoylphosphatidylglycerol (DPPG), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, and lysophosphatidylethanolamine (LPE), or any combination thereof.

In some embodiments, the lipoplex comprises a sterol or a lipid containing sterol moieties (“sterol derivatives”). As defined herein, “sterols” are a subgroup of steroids consisting of steroid alcohols. Exemplary sterols and lipids containing sterol moieties useful in the lipoplex formulations provided herein include, but are not limited to cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In some embodiments, the lipoplex formulation comprises cholesterol.

In some embodiments, the lipoplex formulation further comprises a stabilizing agent, such as one or more surfactants and/or polymer conjugated lipids. Stabilizing agents are a class of molecules which disrupt or help form the hydrophobic-hydrophilic interactions among molecules. Stabilizing agents can be non-ionic or they can be charged. Stabilizing agents that can advantageously be used in the lipoplex formulations provided herein include, but are not limited to, polyethylene glycol (PEG)-modified lipids. Non-limiting examples of PEG-lipids include PEG-modified phosphatidylethanolamine and phosphatidic acid, PEG-ceramide conjugates (e.g., PEG-CerC14, PEG-CerC16, PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid. Other stabilizing agents useful in the compositions disclosed herein include, e.g., polyglycol lipids, polyoxyethylene alkyl ethers, diblock polyoxyethylene ether co-polymers, triblock polyoxyethylene alkyl ethers co-polymers, and amphiphilic branched polymers. In embodiments, a stabilizing agent can be polyoxyethylene (20) oleyl ether, polyoxyethylene (23) lauryl ether, polyoxyethylene (40) stearate (“Myrj52”), poly(propylene glycol)11-block-poly(ethylene glycol)16-block-poly(propylene glycol)11, poly(propylene glycol)12-block-poly(ethylene glycol)28-block-poly(propylene glycol)12, polysorbate 80 (also known as Tween 80, IUPAC name 2-[2-[3,4-bis(2-hydroxyethoxy)oxolan-2-yl]-2-(2-hydroxyethoxy)ethoxy]ethyl octadec-9-enoate), BRIJ™ S10 (Polyoxyethylene (10) stearyl ether), BRIJ™ L4=Polyoxyethylene (4) lauryl ether; BRIJ™ S20=Polyoxyethylene (20) stearyl ether; BRIJ™ S35=Polyoxyethylene (23) lauryl ether; TPGS 1000=D-α-Tocopherol polyethylene glycol 1000 succinate; Tween 20/Polysorbate 80/Tridecyl-D-maltoside in equal ratios, and combinations thereof. The stabilizing agent may be used alone or in combinations with each other.

The lipoplex compositions provided herein can also be combined with one or more exosomes, or biological materials (e.g., lipids, proteins, nucleic acids, or the like) derived or purified from exosomes.

The term “exosome” refers to the small membrane vesicles secreted by most cells that contain cell specific payloads of proteins, lipids and, genetic material and other biomolecules that are transported to other cells in different location of the tissue. Exosomes can be considered liposomal particles. Exosomes or lipid mixtures obtained therefrom, can be used in combination with other transfection agents or helper lipid mixtures. Exosomes are also referred to as microvesicles, epididimosomes, argosomes, exosome-like vesicles, microparticles, promininosomes, prostasomes, dexosomes, texosomes, archeosomes and oncosomes.

Examples of lipid constituents isolated from exosomes include, but are not limited to, Lyso-PC (non-limiting examples of which C-18, C-16, C-14 and mixture), Lyso-bisphospahtidic acid (non-limiting example of which is C-18, C-16 and C-14), sphingomyelin, ceramides (non-limiting examples C-8-C-24), disaturated PC (non-limiting examples DSPC, DPPC, DMPC and others where Cn (n=8-25)), diunsaturated PC-MIX (non-limiting examples of which are DOPC, DP(db)PC) phosphatidyl serine (PS), phosphatidyl inositol (PI)), disaturated PE non-limiting example, DSPE, DPPE, DMPE), di-unsaturated PE-MIX (non-limiting example DOPE DP(db)PE), phosphatidyl glycerol (PG) (non-limiting examples of which are C-18-C-22), cholesterol, and diglycerides, such as cardiolipin.

Also contemplated is the use of any mixtures of combination of the above listed ionizable lipids, helper lipids, exosomes, lipid mixtures isolated from exosomes, and peptides for delivery of a payload into immune cells.

Still other lipoplex formulations may include transfection enhancing agents such as a fusion agent (such as an endosomal release agent), a cell surface ligand and/or a nuclear localization agent such as a nuclear receptor ligand peptide. Examples of transfection enhancing agents include, but are not limited to, reovirus-related fusogenic peptides (e.g., as in WO07/130073, which is hereby incorporated by reference in its entirety), insulin, a transferrin, epidermal growth factor, fibroblast growth factor, a cell targeting antibody, a lactoferrin, a fibronectin, an adenovirus penton base, Knob, a hexon protein, a vesicular stomatitis virus glycoprotein, a Semliki Forest Virus core protein, a influenza hemagglutinin, a hepatitis B core protein, an HIV Tat protein, a herpes simplex virus VP22 protein, a histone protein, a arginine rich cell permeability protein, a high mobility group protein, and invasin protein, and internalin protein, an endotoxin, a diphtheria toxin, a shigella toxin, a melittin, a magainin, a gramicidin, a cecrophin, a defensin, a protegrin, a tachyplesin, a thionin, a indolicidin, a bactenecin, a drosomycin, an apidaecin, a cathelicidin, a bactericidal-permeability-increasing protein, a nisin, a buforin, and fragments thereof and those disclosed in US Application Publication No. 2018/0340188 (which is hereby incorporated by reference in its entirety).

Exemplary transfection enhancing peptides useful in the lipoplex formulations provided herein include, without limitation, those provided in Table 1.

TABLE 1

| SEQ | |
|---|---|
| ID | |
| NO. | Sequence |
| 1 | GYSTPPKKKRKVEDP |
| 2 | GYSTPPKTRRRP |
| 3 | GYSTPGRKKR |
| 4 | GYSTPRRNRRRRW |
| 5 | PDEVKRKKKPPTSYG |
| 6 | PRRRTKPPTSYG |
| 7 | RKKRGPTSYG |
| 8 | WRRRRNRRPTSYG |
| 9 | GYGPPKKKRKVEAPYKA |
| 10 | PAAKRVKLD |
| 11 | RQRRNELKRSP |
| 12 | KRPAATKKAGQAKKKK |
| 13 | VRKKRKTEEESPLKDKDAKKSKQE |
| 14 | RLRRDAGGRGGVYEHLGGAPRRRK |
| 15 | KRKGDEVDGVDECAKKSKK |
| 16 | NQSSNFGPMKGGNFGGRSSGPYGGGGQYFAKPRNQGGY |
| 17 | GGKRTADGSEFESPKKARKVEAYPKAW |
| 18 | GGKRTADGSEFESPKKKRAVEAYPKAW |
| 19 | GGKRTADGSEFESPKKKAKVEAYPKAW |
| 20 | GGKRTADGSEFESPKKKRKVEAPYKAWK |
| 21 | GGKRTADGSEFESPKKKRKVEYKAWK |
| 22 | GYGPAAKRVKLDEAYPKAWK |
| 23 | GGKRTADGSEFEPAAKRVKLDEAYPKAWK |
| 24 | GTGPKKKRKVGGGGYGPKKKRLVG |
| 25 | KRPAATKKAGQAKKKKLEAYPKAWK |
| 26 | ATKGTKRSYEQMETGE |
| 27 | GKWERKPIRCAS |
| 28 | GYGKRTADSQHSTPPKKKRKVEAPYKAWK |
| 29 | KRTADSQHSTPPKKKRKVEAPYKAWK |
| 30 | GYGPPKKKRKVEAPYKAWKWAKYPAMRRA |
| | HHRRRRASHRRRTTTGT |
| 31 | GYGPPKKKRKVEAPYKAWKRGARRYSKMK |
| | RRRRRVARRHRRRP |
| 32 | FWGYGYGPPKKKRKVEAPYKAWK |
| 33 | GKPSSDDEATADSQHSTPPKKKERKVED |
| 34 | GKPTADDQHSTPPKKKRKVED |
| 35 | GGKRTADGSEFESPKKARKVEAYPKAK |
| 36 | EKIRLRPGRKKRYRLKHL |
| 37 | PEGTRQARRNRRRRWRKR |
| 38 | PEGTRQPRRNRRRRWRKR |
| 39 | GVKRSYGAARGDDRRRPNVVAPYKAW |
| 40 | KSVPNRTRTYIKLKRLRFKGAPYKAW |
| 41 | EMRRRREEEGLQLRKQKREEQLFKRRN |
| 42 | FEAALAEALAEALA |
| 43 | Ac-LARLLPRLLARL-NHCH3 |
| 44 | GLLEELLELLEELWEELLEG |
| 45 | GWEGLIEGIEGGWEGLIEG |
| 46 | GLFEALAEFIEGGWEGLIEG |
| 47 | GLFEALLELLESLWELLLEA |
| 48 | GGYCLEKWMIVASELKCFGNTA |
| 49 | GGYCLTRWMLIEAELKCFGNTAV |
| 50 | WEAALAEALAEALAEHLAEALAEALEALAA |
| 51 | GLFGAIAGFIENGWEGMIDGWYG |
| 52 | GIGAVLKVLTTGLPALISWIKRKRQQ |
| 53 | GRKKRRQRRRPPQ |
| 54 | RQIKIWFQNRRMKWKK |
| 55 | GWTLNSAGYLLGKINLKALAALAKKIL |
| 56 | WEAKLAKALAKALAKHLAKALAKALKACEA |
| 57 | GLFKALLKLLKSLWKLLLKA |
| 58 | GLFRALLRLLRSLWRLLLRA |
| 59 | GLFEALLELLESLYELLLEA |
| 60 | GLFEALEELWEA |
| 61 | GLFLLEEWLE |
| 62 | GLFLLEEWLEK |
| 63 | GLFEALLELLESLWELLLEAK |
| 64 | Suc-GLFKLLEEWLE |
| 65 | Suc-GLFKLLEEWLEK |
| 66 | GLFEAIAEFIEGGWEGLIEG |
| 67 | GLFKAIAKFIKGGWKGLIKG |
| 68 | IRFKKTKLIASIAMALC |
| 69 | ALAGTIIAGASLTFQVLDKVIEELGKVSRK |
| 70 | GLFEAIEGFIENGWEGMIDGWYG |
| 71 | GYICRRARGDNPDDRCT |
| 72 | GLFEAIAEFIEGGWEGLIEGCA |
| 73 | GLFHAIAHFIHGGWHGLIHGWWYG |
| 74 | RRRQRRKKRGGDIMGEWGNEIFGAIAGFLG |
| 75 | GLFEAIADFIENGWEGMIDGGG |
| 76 | ALAGTIIAGASLTFQVLDKVIEELGKVSRKK |
| 77 | IRFKKTKLIASIAMA |
| 78 | GLWHLLLHLWRRLLRLLR |
| 79 | KKIMLLLMTLLLVSLPLAQEQ |
| 80 | GLFEALLELLESLWELLLEAWYG |
| 81 | RLLRLLLRLWRRLLRLLR |
| 82 | LLELELLELELLLELELLELELLLEL |
| 83 | GLFEALLELLESLWELLLEARRRRRRRR |
| 84 | GLFEALLELLESLWELLLEARRRRRR |
| 85 | GLFEALLELLESLWELLLEAKKKKKKKK |
| 86 | GLFEALLELLESLWELLLEAKKKKKK |
| 87 | GLFEALLELLESLWELLLEAKK |
| 88 | GLFEALLELLESLWELLLEAKKKK |
| 89 | GLFEALLELLESLWELLLEAEE |
| 90 | GLFEALLELLESLWELLLEAEEEE |
| 91 | GLFEALLELLESLWELLLEAEEEEEE |
| 92 | GLFEALLELLESLWELLL |
| 93 | PLSSIFSRIGDPRGARRYAKMKRRR |
| | RRVARRHRRRP |
| 94 | GPFHYFQFLFPPV |
| 95 | GSSSWWQRWWPPW |
| 96 | RRRQRRKKR |
| 97 | KKKK |
| 98 | KKKKKK |
| 99 | KKKKKKKK |
| 100 | KKKKKKKKKK |
| 101 | KKKKKKKKKKKK |
| 102 | KKKKKKKKKKKKKKKK |
| 103 | KKKKKKKKKKKKKKKKKKKK |
| 104 | KKKKKKKKKKKK |
| 105 | RRRR |
| 106 | RRRRRR |
| 107 | RRRRRRRR |
| 108 | RRRRRRRRRR |
| 109 | RRRRRRRRRRRR |
| 110 | RRRRRRRRRRRRRRRR |
| 111 | RRRRRRRRRRRRRRRRRRRR |
| 112 | RRRRRRRRRRRRRRRRRRRRRRRR |
| 113 | YKA |
| 114 | KKKKKKKKWKGGGGACYGLPHLFCG |
| 115 | YKAKKKKKKKKWK |
| 116 | KTPKKAKKPKTPKKAKKP |
| 117 | KKAKKPAATRKSSKNPKKPKTVKPKKVAK |
| 118 | RGARRYSKMKRRRRRVARRHRRRP |
| 119 | TRQARRNRRRRWRERQRGSGSG |
| 120 | KRPRGRPKGSKKNWRRRKRRASRRSPRRR |
| 121 | KRGRGRPRKQPPKEPSEVPTPKRPRGRPKGSKNK |
| 122 | KEKYEKDIAAYRAKGKPAAKKGVVKAEKSKKKK |
| 123 | YKAKKKKKKKKKKWK |
| 124 | KKKKKKKGGC |
| 125 | YRARRRRRRRRWR |
| 126 | YRARRRRRRRRRRWR |
| 127 | KGDPKKPRGKMSSYAFFVQTCREEHKKKHPD |
| | ASVNFSEFSKK |
| 128 | KKQLKKQLKKQLKQWK |
| 129 | KKSPKKSPKKSPKKSK |
| 130 | KLSKLEKKSKLEK |
| 131 | KLSKLEKKLSKLEKKSKLEK |
| 132 | KSLKKSLKKSLKKSK |
| 133 | KIRRRGKNKVAARTCRQRRTDR |
| 134 | KIRRRGKNKVAAQNCRKRKLET |
| 135 | KRRIRREKNKMAAAKCRNRRRELT |
| 136 | KDRSNLLERHTR |
| 137 | KRPAATKKAGQAKKKL |
| 138 | RRRRRREEEE |
| 139 | RRRRRREEEEEE |
| 140 | RRRRRREEEEEEEE |
| 141 | RRRRRRRREEEE |
| 142 | RRRRRRRREEEEEE |
| 143 | RRRRRRRREEEEEEEE |
| 144 | RRRRRRRRRRRREEEE |
| 145 | RRRRRRRRRRRREEEEEE |
| 146 | RRRRRRRRRRRREEEEEE |
| 147 | KLSKLEKK |
| 148 | SKLEK |
| 149 | KLSKLEKKLSKLEKK |
| 150 | PKKKRKVGGGRGDSP |
| 151 | LPHKSMPCG |
| 152 | GACLQHKSMPCG |
| 153 | YGLPHLFCG |
| 154 | SERSMNFCG |
| 155 | DHYSLYEDLERGTDK |
| 156 | ISLPRTSGAQRASTTR |
| 157 | EKLQTKYGLPHKVEFCG |
| 158 | TRISESQAKPGD |
| 159 | LVFFDY |
| 160 | WGGNGPTTFDCSGYTKYVFAK |
| 161 | INIGTTGWGDHYSLY |
| 162 | YDNIHG |
| 163 | AGWGKFLVGFGRV |
| 164 | SIGYPLP |
| 165 | TTHWGFTL |
| 166 | HLQIQPYPQISG |
| 167 | KLNIVSVNG |
| 168 | RGH |
| 169 | DNRIRLQAKAA |
| 170 | KIKMVISWKG |
| 171 | LPWYSYLYAVSA |
| 172 | WNLPWYYSVSPT |
| 173 | WNL |
| 174 | PWYYSVSPT |
| 175 | SSWESYKSGGGTRL |
| 176 | RDWSSQHPGRCNGETHLK |
| 177 | SLPTLTL |
| 178 | VICTGGDYSFALPVGQWPVMT |
| 179 | DKPSYQFGGHNSVDFEEDTLPKV |
| 180 | RARRRKRASATQLYQTCKASGTCPPD |
| 181 | SGDYSFALPVGQWPWMTG |
| 182 | CTGGDYSFALPVGQWPW |
| 183 | FYYDYDFFFDYWGQG |
| 184 | HLRRLRRRLLREAEG |
| 185 | DYYCAAWDDSLNGYSVF |
| 186 | YYCLQSMEDPYTFGG |
| 187 | YYCARSDGNYGYYYALDYDY |
| 188 | AARSPSYYRYDY |
| 189 | GPYYAMDYD |
| 190 | YYCQQRSSYPYTEGGAYPKAWK |
| 191 | YYCQRYDSDWSFGQGTKL |
| 192 | YYCARSGYYAMDYWGQGT |
| 193 | RVRRGACRGDCLG |
| 194 | RVRRGACRYDCLG |
| 195 | YYCAKGTHWGFWSGYFDYWGQGT |
| 196 | GRENYHGCTTHWGFTLC |
| 197 | VQATQSNQHTPRGGGSK |
| 198 | DPRAPGS |
| 199 | YYCQQRSSYPYTFGG |
| 200 | AARSPSYYRYDYGPYYAMDYD |
| 201 | GPKLTGILISILSLFVES |
| 202 | KYILRWRPKNS |
| 203 | IKVAV |
| 204 | WTPPRAQITGYRLTVGLTRR |
| 205 | AASIKVAVSADR |
| 206 | KLDAPT |
| 207 | NRWHSIYITRFG |
| 208 | PHSRN |
| 209 | SSFHFDGSGYAM |
| 210 | RGDS |
| 211 | IAFQRN |
| 212 | GRGDSP |
| 213 | TWYKIAFQRRK |
| 214 | EDGIHEL |
| 215 | SLVRNRRVITIQ |
| 216 | YRVRVTPKEKTGPMKE |
| 217 | LQVQLSR |
| 218 | SPPRRARVT |
| 219 | RKRLQVQLSIRT |
| 220 | ATETTITIS |
| 221 | NAPFPKLSWTIQ |
| 222 | VSPPRRARVTDATETTITISWRTKTETITGG |
| 223 | WTIQTTVDRGLL |
| 224 | KPDVRSYTITG |
| 225 | DTINNGRDHMILI |
| 226 | ANGQTPIQRYIK |
| 227 | MILISIGKSQKRM |
| 228 | PRARITGYIIKYEKPGSPPREVVPRPRPGV |
| 229 | PPFLMLLKGSTR |
| 230 | WQPPRARI |
| 231 | NQRLASFSNAQQS |
| 232 | WQPPRARITGYIIKYEKPG |
| 233 | ISNVFVQRMSQSPEVLD |
| 234 | YEKPGSPPREVVPRPRPGV |
| 235 | KARSFNVNQLLQD |
| 236 | KNNQKSEPLIGRKKT |
| 237 | KNSFMALYLSKG |
| 238 | EILDVPST |
| 239 | KNSFMALYLSKGRLVFALG |
| 240 | IDAPS |
| 241 | RDSFVALYLSEGHVIFAGLG |
| 242 | VVIDASTAIDAPSNL |
| 243 | KPRLQFSLDIQT |
| 244 | LDVPS |
| 245 | DGQWHSVTVSIK |
| 246 | REDV |
| 247 | FVLYLGSKNAKK |
| 248 | PHSRNRGDSP |
| 249 | LAIKNDNLVYVY |
| 250 | LWVTVRSQQRGLF |
| 251 | AYFSIVKIERVG |
| 252 | GTNNWWQSPSIQN |
| 253 | DVISLYNFKHIY |
| 254 | WVTVTLDLRQVFQ |
| 255 | FFDGSSYAVVRD |
| 256 | RQVFQVAYIIIKA |
| 257 | LHVFYDFGFGFSNG |
| 258 | LTRYKITPRRGPPT |
| 259 | LKKAQINDAKYREISIIYHN |
| 260 | LLEFTSARYIRL |
| 261 | RAYFNGQSFIAS |
| 262 | YIRLRLQRIRTL |
| 263 | SRLRGKNPTKGK |
| 264 | RRYYYSIKDISV |
| 265 | LHKKGKNSSKPK |
| 266 | SINNTAVNQRLT |
| 267 | RLKTRSSHGMIF |
| 268 | GGFLKYTVSYDI |
| 269 | GEKSQFSIRLKT |
| 270 | RDQLMTVLANVT |
| 271 | TLFLAHGRLVFM |
| 272 | ANVTHLLIRANY |
| 273 | LVFMFNVGHKKL |
| 274 | AGTFALRGDNPQG |
| 275 | TLFLAHGRLVFMFNVGHKKL |
| 276 | VLIKGGRARKHV |
| 277 | DFMTLFLAHGRLVFMGNVG |
| 278 | LSNIDYLIKAS |
| 279 | HKKLKIRSQEKY |
| 280 | LQQSRIANISME |
| 281 | GAAWKIKGPIYL |
| 282 | NLLLLLVKANLK |
| 283 | VIRDSNVVQLDV |
| 284 | HRDELLLWARKI |
| 285 | GLIYYVAHQNQM |
| 286 | KRRARDLVHRAE |
| 287 | DYATLQLQEGRLHFMFDLG |
| 288 | SQFQESVDNITK |
| 289 | KKGSYNNIVVHV |
| 290 | PGGMREKGRKAR |
| 291 | ADNLLFYLGSAK |
| 292 | MEMQANLLLDRL |
| 293 | GSAKFIDFLAIE |
| 294 | LSEIKLLISAR |
| 295 | KVSFLWWVGSGV |
| 296 | RDFTKATNIRLRFLR |
| 297 | SYWYRIEASRTG |
| 298 | ISTVMFKFRTFS |
| 299 | YFDGTGFAKAVG |
| 300 | KQANISIVDIDSN |
| 301 | NGQWHKVTAKKI |
| 302 | FSTRNESGIILL |
| 303 | AKKIKNRLELVV |
| 304 | RRQTTQAYYAIF |
| 305 | GFPGGLNQFGLTTN |
| 306 | YAIFLNKGRLEV |
| 307 | NQFGLTTNIRFRG |
| 308 | KNRLTIELEVRT |
| 309 | IRSLKLTKGTGKP |
| 310 | GLLFYMARINHA |
| 311 | AKALELRGVQPVS |
| 312 | VQLRNGFPYFSY |
| 313 | GQLFHVAYILIKF |
| 314 | HKIKIVRVKQEG |
| 315 | NVLSLYNFKTTF |
| 316 | DFGTVQLRNGFPFFSYDLG |
| 317 | SQRIYQFAKLNYT |
| 318 | NIRLRFLRTNTL |
| 319 | EVNVTLDLGQVFH |
| 320 | GKNTGDHFVLYM |
| 321 | GQVFHVAYVLIKF |
| 322 | VVSLYNFEQTFML |
| 323 | HQQDLGTAGSCLRKFSTMFLF |
| 324 | RFDQELRLVSYN |
| 325 | HQQDLGTAGSCLRKFSTMFLFCNI |
| 326 | RLVSYSGVLFFLK |
| 327 | VAEIDGIEL |
| 328 | NWRHISYITRFG |
| 329 | GIIFFL |
| 330 | KRLQVQLRSIRT |
| 331 | ASKAIQVFLLGG |
| 332 | TWYKIAFQRNRK |
| 333 | VLVRVERATVFS |
| 334 | QVFQVAYIIIKA |
| 335 | TVFSVDQDNMLE |
| 336 | GEFYFDLRLKGDK |
| 337 | RLRGPQRVFDLH |
| 338 | GTPGPQGIA |
| 339 | FDLHQNMGSVN |
| 340 | GQRDVV |
| 341 | LRAHAVDVNG |
| 342 | TAGSCLRKFSTM |
| 343 | LFSHAVSSNG |
| 344 | KGHRGF |
| 345 | TAGSCLRKFSTMFLF |
| 346 | TAGSCLRKFSTMFLFCNI |
| 347 | DLGTAGSCLRKFSTM |
| 348 | HQQDLGTAGSCLRKFSTM |
| 349 | RNIAEIIKDI |
| 350 | SIGFRGDGQTC |
| 351 | LNRQELFPFG |
| 352 | RIQNLLKITNLRIKFVK |
| 353 | KKQRFRHRNRKGYRSQ |
| 354 | SINNTAVMQRLT |
| 355 | FRHRNRKGY |
| 356 | RYRVRVTPKEKTGPMKE |
| 357 | SETTVKYIFRLHE |
| 358 | GHRGPTGRPGKRGKQGQKGDS |
| 359 | KAFDITYVRLKF |
| 360 | GDLGRPGRKGRPGPP |
| 361 | YIGSR |
| 362 | RGEFYFDLRLKGDK |
| 363 | LAGSCLARFSTM |
| 364 | LALFLSNGHFVA |
| 365 | ISRCQVCMKKRH |
| 366 | PGRWHKVSVRWE |
| 367 | TDIPPCPHGWISLWK |
| 368 | VRWGMQQIQLVV |
| 369 | TAIPSCPEGTVPLYS |
| 370 | KMPYVSLELEMR |
| 371 | GPAGKDGEAGAQG |
| 372 | VLLQANDGAGEF |
| 373 | GLPGER |
| 374 | DGRWHRVAVIMG |
| 375 | LAGSCLPVFSTL |
| 376 | APVNVTASVQIQ |
| 377 | TAGSCLRRFSTM |
| 378 | KQGKALTQRHAK |
| 379 | TAGSCLRKF |
| 380 | RYVVLPR |
| 381 | TAGSCL |
| 382 | SPYTFIDSLVLMPY |
| 383 | TAG |
| 384 | PDSGR |
| 385 | QQNLGSVNVSTG |
| 386 | SRATAQKVSRRS |
| 387 | DPGYIGSR |
| 388 | GSLSSHLEFVGI |
| 389 | VILQQSAADIAR |
| 390 | RNRLHLSMLVRP |
| 391 | KDISEKVAVYST |
| 392 | APMSGRSPSLVLK |
| 393 | LGTIPG |
| 394 | AFGVLALWGTRV |
| 395 | TDIRVTLNRLNTF |
| 396 | IENVVTTFAPNR |
| 397 | AFSTLEGRPSAY |
| 398 | LEAEFHFTHLIM |
| 399 | TSAEAYNLLLRT |
| 400 | HLIMTFKTFRPA |
| 401 | LNRRYEQARNIS |
| 402 | KTWGVYRYFAYD |
| 403 | SLLSQLNNLLDQ |
| 404 | TNLRIKFVKLHT |
| 405 | RDIAEIIKDI |
| 406 | KRLVTGQR |
| 407 | SHAVSS |
| 408 | GPGVVVVERQYI |
| 409 | ADTPPV |
| 410 | NEPKVLKSYYYAI |
| 411 | LRAHAVDING |
| 412 | YYAISDFAVGGR |
| 413 | DSITKYFQMSLE |
| 414 | LPFFNDRPWRRAT |
| 415 | YTALIIATDN |
| 416 | FDPELYRSTGHGGH |
| 417 | VITVKDINDN |
| 418 | TNAVGYSVYDIS |
| 419 | GLDRESYPYY |
| 420 | APVKFLGNQVLSY |
| 421 | MKVSATDADD |
| 422 | SFSFRVDRRDTR |
| 423 | PQVTRGDVFTMP |
| 424 | TWSKVGGHLRPGIVQSG |
| 425 | KEAEREVTDLLR |
| 426 | RGDV |
| 427 | AAEPLKNIGILF |
| 428 | FALWDAIIGEL |
| 429 | VGVAPG |
| 430 | LWPLLAVLAAVA |
| 431 | PGVGV |
| 432 | VFDNFVLK |
| 433 | TSIKIRGTYSER |
| 434 | TTSWSQCSKS |
| 435 | DPETGV |
| 436 | KRSR |
| 437 | QGADTPPVGV |
| 438 | SVVYGLR |
| 439 | PLDREAIAKY |
| 440 | DGRGDSVAYG |
| 441 | HAVDI |
| 442 | LALERKDHSG |
| 443 | DQNDN |
| 444 | YSMKKTTMKIIPFNRLTIG |
| 445 | QDPELPDKNM |
| 446 | RGDF |
| 447 | LVVQAADLQG |
| 448 | GVYYQGGTYSKAS |
| 449 | NDDGGQFVVT |
| 450 | TAGSCLRKFSCL |
| 451 | YILHVAVTN |
| 452 | CNYYSNSYSFWLASLNPER |
| 453 | TYRIWRDTAN |
| 454 | TGLSCLQRFTTM |
| 455 | GFTCECSIGFRGDGQTCYGIVFWSEV |
| 456 | HHLGGAKQAGDV |
| 457 | SCLPGFSGDGRACRDVDECGH |
| 458 | MAPRPSLAKKQRFRHRNRKGYRSQRGHSRG |
| 459 | KKQKFRHRNRKGYRSQ |
| 460 | KKQKFKHRNRKGYRS |
| 461 | KKQKFRRRNRKGYRSH |
| 462 | TAIPPCPHGWISLWK |
| 463 | KKQKSRHRSRKRYRS |
| 464 | KKQKSRRRSRKGYRS |
| 465 | ISRCTVC |
| 466 | ISRCQVCMKRRH |
| 467 | VSRCTVC |
| 468 | TDIPPCPQGWISLWK |
| 469 | TVKAGELEKIISRCQVMKKRH |
| 470 | TDIPSCPHGWISLWK |
| 471 | TDIPPCPAGWISLWK |
| 472 | TEIPPCPQGWISLWK |
| 473 | TDVPPCPQGWISLWK |
| 474 | RLVSYNGILFFLK |
| 475 | RLVSYSGVIFFLK |
| 476 | RLVSYNGILFFL |
| 477 | RLVSYSGIIFFLK |
| 478 | RFEQELRLVSYSGVLFFLKQ |
| 479 | RLVSYNGIIFFLK |
| 480 | DPAFKIEDPYSPRIQNLLKITNLRIKFVKL |
| 48 | TKRFEQELRLVSYSGVLFFL |
| 482 | GGRLKYSVAF |
| 483 | GGFLRYTVSYDI |
| 484 | GGFLKYTVSYDV |
| 485 | LGNKLTAFGGFLKYTVSYDIPV |
| 486 | GGYLKYTVSYDI |
| 487 | GEIFFDMRLKGDK |
| 488 | GEIYFDLRLKGDK |
| 489 | GEIYLDMRLKGDK |
| 490 | IGQPGAKGEPGEFYFDLRLKGDKGDPGFPG |
| 49 | GEVFFDMRLKGDK |
| 492 | LAGSCLPIFSTL |
| 493 | AHNQDLGLAGSCLARFSTMPFLYCNPGDIC |
| 494 | QEKAHNQDLGLAGSCLPVFSTLPFAYCNIH |
| 495 | LAGSCLPVFSTM |
| 496 | GNKRAHGQDLGTAGSCLRRFSTMPFMFCNI |
| 497 | RAHGQDLGTAGSCLRRFSTMP |
| 498 | RKRLQVQLNIRT |
| 499 | HLVLPLQQSDVRKRLQVQLSIRTFASSGLI |
| 500 | RKRLSVQLRIRT |
| 501 | DLGTAGSCLRRFSTM |
| 502 | RNIAEIIKDI |
| 503 | TAGSCLRKFSTMRRRRRRRRRRRR |
| 504 | FTLTGLLGTLVTMGLLT |
| 505 | APYKAWK |
| 506 | STSKTNRGDDSNWSKRVTNNKPS |
| 507 | STSKRKRGDDSNWSKRVTKKKPS |
| 508 | STSKRKRGDDSNWSKRVSKKKPS |
| 509 | STSKRKRGDDANWSKRVTKKKPS |
| 510 | PLAGSKRKRADEVAWSKRGTKKKPER |
| 511 | PLAGSKRKRADEVAWSKRGTKKKPER |
| | TSAARAGPSRRIR |
| 512 | STSKRKRGDDANWSKRTTKKKPSS |
| 513 | STSKRKRGDDANWSKRTTKKKPSSAG |
| | LKRAGSKADRPSL |
| 514 | PTTAGKRKRSDDAAWSKRARPKAGRT |
| 515 | PTTAGKRKRSDDAAWSKRARPKAGRT |
| | SAARPGTSVRRIR |
| 516 | SSSLGKRKRSDEGAWSKGKSKKKAMR |
| 517 | SSSLGKRKRSDEGAWSKGKSKKKAMR |
| | GSSSRRPGPVRGP |
| 518 | PTTAGKRKRTDDAAWSKRARPKAGR |
| 519 | PTTAGKRKRTDDAAWSKRARPKAGRT |
| | SAARPGTAVRRVR |
| 520 | PATAGKRKRSDDAAWSKRARPKAGRT |
| | SAAR |
| 521 | PATAGKRKRSDDAAWSKRARPKAGRT |
| | SAARPGTSVRRIR |
| 522 | SSSLGKRKRSNGGDWSKRSAKKKPA |
| 523 | SSSLGKRKRSNGGDWSKRSAKKKPAG |
| | TPSRRAGPGRGPR |
| 524 | SSSLGKRKRSDEGAWSKGKSKKKAMR |
| 525 | SSSLGKRKRSDEGAWSKGKSKKKAMRG |
| | SSSRRPGPVRGP |
| 526 | STSKRKRGDDANWNKRPTKKKPSS |
| 527 | STSKRKRGDDANWNKRPTKKKPSSAG |
| | LKKAGSKAERPSL |
| 528 | SGALKRKRSDEVAWSRRRPVKKPV |
| 529 | SGALKRKRSDEVAWSRRRPVKKPVRR |
| | APPPRAGPSVRRG |
| 530 | SGALKRKRSDEVAWSRRKPAKKPAR |
| 531 | SGALKRKRSDEVAWSRRKPAKKPARQ |
| | PPPPRAGPSVRRG |
| 532 | AGALKRKRSDEVAWSRRKPAKKPAR |
| 533 | AGALKRKRSDEVAWSRRKPAKKPARAPPPR |
| | AGPSVRRGL |
| 534 | STSKRKRGDDSNWSKRVTKKKPSSAGLKRA |
| | GSKADRPSLQIQTLQHAGTTMITVPSGGVC |
| | DLINTYARGSDEGNRHTSETLTYKIAIDYH |
| | FVADAAACRYSNTGTGVMWLVYDTTPGGQA |
| | PTPQTIFSYPDTLKAWPATWKVSRELCHRF |
| | VVKRRWLFNMETDGRIGSDIPPSNASWKPC |
| | KRNIYFHKFTSGLGVRTQWKNVTDGGVGAI |
| | QRGALYMVIAPGNGLTFTAHGQTRLYFKSV |
| | GNQ |
| 535 | DPQNALYYQPRVPTAAPTSGGVPWSRVGEV |
| | AILSFVALICFYLLYLWVLRDLILVLKARQ |
| | GRSTEELIFGGQAVDRSNPIPNIPAPPSQG |
| | NPGPFVPGTG |
| 536 | GSQLVPPPSAFNYIESQRDEFQLSHDLTEI |
| | VLQFPSTASQITARLSRSCMKIDHCVIEYR |
| | QQVPINASGTVIVEIHDKRMTDNESLQASW |
| | TFPIRCNIDLHYFSSSFFSLKDPIPWKLYY |
| | RVSDSNVHQMTHFAKFKGKLKLSSAKHSVD |
| | IPFRAPTVKILAKQFSEKDIDFWHVGYGKW |
| | ERRLVKSASSSRFGLRGPIEINPGESWATK |
| | SAIVTPNRNADLDIEEELLPYRELNRLGTN |
| | ILDPGESASIVGIQRSQSNITMSMSQLNEL |
| | VRSTVHECIKTSCIPSTPKSLS |
| 537 | RTGVKRSYGAARGDDRRRPNVV |
| 538 | SYVKTVPNRTRTYIKLRVR |
| 539 | MYSTSNRRGRSQTQRGSHVRRTGVKRSYGA |
| | ARGDDRRRPNVVSKTQVEPRMTIQRVQENQ |
| | FGPEFVLSQNSALSTFVTYPSYVKTVPNRT |
| | RTYIKLKRVRFKGTLKIERGQGDTIMDGPS |
| | SNIEGVFSMVIVVDRKPHVSQSGRLHTFDE |
| | LFGARIHCHGNLSVVPALKDRYYIRHVTKR |
| | VVSLEKDTLLIDLHGTTQLSNKRYNCWASF |
| | SDLERDCNGVYGNITKNALLVYYCWLSDAQ |
| | SKASTYVSFELDYLG |
| 540 | RRRRRRRRRRRRVDYGKWERKPIRCASMSR |
| 541 | RRRRRRRRRRRRGKWERKPIRCAS |
| 542 | KKKKKKKKKKKKKKKKGKWERKPIRCAS |
| 543 | RRRRRRRRRRRRVDFSHVDYGKWERKPIR |
| | CASMSRLGLRG |
| 544 | GVKRSYGAARGDDRRRPNVVAPYKAWRRR |
| | RRRRRRRRR |
| 545 | KSVPNRTRTYIKLKRLRFKGAPYKAWRRR |
| | RRRRRRRRR |
| 546 | RTGVKRSYGAARGDDRRRPNVVRRRRRRR |
| | RRRRR₂ |
| 547 | SYVKTVPNRTRTYIKGGGGGRRRRRRRRR |
| | RRR₂ |
| 548 | VDIPFRAPTIKILSKQFTEDDIDFWHVGY |
| | GKWERKLVRPASLSGRRGLRR |
| 549 | IDFWHVGYGKWERKLVRPASLSGRRGLRR |
| 550 | IDFWSVEKGETRRRLLNPTPHAHSPRPIA |
| | HR |
| 551 | IDFSHVGYGKWERKMIRSASISRLGLHN |
| 552 | VDFSHVGYGKWERKLIRSASTVKYGLPS |
| 553 | IDFSHVDYGKVERKLVKCESSSRLGLHS |
| 554 | IDFWSVGRKAQQRKLVQGPSLIGSRSMRY |
| 555 | IDFWSVGSKPQTRRLVDGSRLIGHSSRSLRV |
| 556 | IDFWSVERGETRRRLLNPTPSAGSNRALSKR |
| 557 | VDFWSVGKPKPIRRLIQNDPGTDYDTGPKYR |
| 558 | VDFWSVEKPKPIRRLLNPGPNQGPYPNTGHR |
| 559 | VDFSHVDYGKWERKLIRSASTSRYGLRS |
| 560 | VDFSHVDYGKWERKTLRSRSLSRIGLTG |
| 561 | IDFWHVGYGKWERRLVKSASSSRFGIRG |
| 562 | VDFFHVDYGRWERKHIRCASMSRVGLRG |
| 563 | GTFQHVDYGKWERKPIRCQSMSRVGYRR |
| 564 | VGYGKWERKLVRPASLS |
| 565 | VEKGETRRRLLNPTPHA |
| 566 | VGYGKWERKLIRSASTV |
| 567 | VEKPKPIRRLLNPGPNQ |
| 568 | VDYGKWERKLIRSASTS |
| 569 | VDYGKWERKTLRSRSLS |
| 570 | VGYGKWERRLVKSASSS |
| 571 | VDYGRWERKHIRCASMS |
| 572 | VERPKPIRRLLTPTPGC |
| 573 | PFRAPTIKILSKQFTEDDIDFWHVGYGKWE |
| | RKLVRPASLSGRRGLRR |
| 574 | PFRAPTVKILSKQFTDKDIDFSHVGYGKWE |
| | RKMIRSASISRLGL |
| 575 | DIAFRAPTVKILSKQFTDRDVDFSHVGYGK |
| | WERKLIRSASTVKYGL |
| 576 | DIRFKPPTINILSKDYTADCVDFWSVEKPK |
| | PIRRLLNPGPNQGPYPNTG |
| 577 | DIPFRAPTVKIHSKQFSHRDVDFSHVDYGK |
| | WERKTLRSRSLSRIGL |
| 578 | DIPFRAPTVKILAKQFSEKDIDFWHVGYGK |
| | WERRLVKSASSSRFGI |
| 579 | DIPFRAPTVKILSKQFTDKDVDFFHVDYGR |
| | WERKHIRCASMSRVGL |
| 580 | DIKYKPPTIKILSKDYTADCVDFWSVERPK |
| | PIRRLLTPTPGCG |
| 581 | ARTKQTARKSTGGKAPRKQLATKAARKSAP |
| | ATGGVKKPHRYRPGTVA |
| 582 | SGRGKGGKGLGKGGAKRHRKVLRDNIQGIT |
| | KPAI |
| 583 | GRKKRRQRRR |
| 584 | GYGPPKKKRKVEAPYKA(K)₂₀₋₄₀K |

In some examples, the transfection enhancing peptide has at least 80%, at least 85%, or least 90% sequence identity to any one of SEQ ID NOs of Table 1. In other examples, the transfection enhancing peptide has at least 9500 sequence identity to any one of SEQ ID NO: 1-584. In other examples, the transfection enhancing peptide has 100% sequence identity to any one of SEQ ID NO: 1-584. In some embodiments, any of the peptides of Table 1 may further comprise a polycationic nucleic acid binding sequence at the N or/and C termini. In some embodiments, any of the peptides of Table 1 may further comprise additional arginine residues (e.g., R2, R4, R8, R12) or lysine residues (e.g., K4, K6, K8, K12) at the C terminus.

In lipoplex formulations provided herein comprising at least one transfection enhancing peptide, the peptide(s) can be present in the formulation at a concentration from about 0.001 to about 0.5 mg/ml. In embodiments, the peptide is at a concentration from about 0.001 to about 0.05 mg/ml, from about 0.01 to about 0.1 mg/mL, or from about 0.05 to about 0.5 mg/mL.

In some embodiments, the lipoplex comprises at least one ionizable lipid having structure I and one or more helper lipids. As described herein, the one or more helper lipids may be selected from other ionizable lipids, cationic lipids, neutral lipids, phospholipids, sterols, sterol derivatives, or combinations thereof. In certain embodiments, the lipoplex comprises at least one ionizable lipid having structure I, one or more helper lipids, and a stabilizing agent.

In lipoplex formulations provided for delivering a payload into an immune cell, the ionizable lipid can be present at about 10 to about 70 mol % of the overall lipid formulation. In some embodiments, ionizable lipid(s) are present from about 15-50 mol %, e.g., about 20-40 mol %. In some embodiments, ionizable lipid(s) are present from about 30-70 mol %, e.g., about 40-60 mol %. In certain embodiments, the amount of the ionizable lipid in the lipoplex composition disclosed herein is at least about 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 mol % of the overall lipid formulation.

In lipoplex formulations provided herein comprising at least one helper lipid, the helper lipid(s) can be present at about 30-90 mol % of the overall lipid formulation. In some embodiments, the helper lipid(s) are present from about 40-70 mol %, e.g., about 50-70 mol %. In some embodiments, the helper lipid(s) is present from about 60-90 mol %, e.g., about 70-90 mol %. In certain embodiments, the amount of helper lipid in the lipoplex composition disclosed herein is at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 mol % of the overall lipid formulation.

In some embodiments, the helper lipid(s) present at about 30-90 mol % of the overall lipid content of the lipoplex formulation is a neutral lipid(s). In some embodiments, the helper lipid(s) present at about 30-90 mol % of the overall lipid formulation is a phospholipid(s). In some embodiments, the helper lipid(s) present at about 30-90 mol % of the overall lipid formulation is a sterol(s) or sterol derivative(s). In some embodiments, the helper lipids present at about 30-90 mol % of the overall lipid formulation is a combination of phospholipid(s) and sterol(s) or sterol derivative(s). Some lipoplex formulations provided herein do not include a sterol or sterol derivative.

In lipoplex formulations provided herein comprising at least one stabilizing agent, the stabilizing agent(s) can be present at about 0.1-10 mol % of the overall lipid formulation.

In certain compositions, the stabilizing agent(s) is present at about 0.1-5 mol % of the lipid composition. In some compositions, the stabilizing agent(s) is present at about 1.0-10 mol % of the lipid composition. For example, in some lipoplex compositions, the stabilizing agent is present at about 0.1, 0.25, 0.5, 1.0, 1.5, 2.0, 2.5, 3.0, 3.5, 4.0, 4.5, 5.0, 6.0, 7.0, 8.0, 9.0, 10 mol %, or any value in between, of the overall lipid formulation. In some examples, the stabilizing agent is present at about 0.5-5 mol % of the overall lipid formulation. In other examples, the stabilizing agent is present at about 1.0-6.0 mol % of the overall lipid formulation.

Accordingly, some lipid complex formulations include a lipid of structure I, or a combination of structure I and one or more cationic/ionizable lipids at 15-60 mol %, a sterol at 20-60 mol %, a stabilizing agent at 0.5-5 mol %, and a phospholipid at 20-60 mol % of the lipid complex formulation. An exemplary lipid complex formulation can include about 30-70 mol % structure I lipid or combination of structure I lipid and one or more additional cationic/ionizable lipids, about 10-30 mol % phospholipid, about 10-30 mol % sterol or sterol derivative; and about 0.5-10 mol % stabilizing agent. Another exemplary lipid complex formulation includes a about 15-50 mol % lipid of structure I or combination of lipid of structure I and one or more cationic/ionizable lipids, about 1-10 mol % stabilizing agent, about 10-30 mol % sterol or sterol derivative, and about 20-60 mol % phospholipid.

In some embodiments of the lipoplex compositions, the payload is one or more nucleic acids (e.g. mRNAs, siRNAs, sgRNAs) and amounts thereof may be selected to provide a specific N/P ratio. The N/P ratio of the composition refers to the molar ratio of ionizable (in physiological pH) nitrogen atoms in one or more lipids to the number of phosphate groups in a nucleic acid (e.g., an RNA). For example, Schoenmaker et al (International Journal of Pharmaceutics; 601 (2021), incorporated herein by reference in its entirety) discusses RNA-lipid nanoparticle N/P ratios (eg, mRNA and siRNA payloads) for example at Table 1 and p. 4.

In certain embodiments, the one or more nucleic acids (e.g. mRNAs, siRNAs, sgRNAs), lipids, and amounts thereof may be selected to provide an N/P ratio from about 2.0 to about 8.0, such as 2, 3, 4, 5, 6, 7, and 8. In some embodiments, the N/P ratio is from 0.05 to 2.0. In other embodiments, the one or more nucleic acids, lipids, and amounts thereof may be selected to provide an N/P ratio from about 5 to about 60, such as 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, or 60. In certain embodiments, the N/P ratio may be from about 5 to about 10. In other embodiments, the N/P ratio is from about 5 to about 20. In other embodiments, the N/P ratio may be from about 10 to about 20, about 10 to about 30, about 15 to about 30, about 15 to about 40, about 20 to about 30, about 20 to about 40, about 20 to about 50, about 30 to about 50, about 30 to about 40, or about 35 to about 50.

Lipoplex formulations for use in the provided methods may be prepared by various methods used in transfection and in in vivo administration. One of the simplest methods for formulation is reverse evaporation, as described in U.S. Pat. No. 9,259,475, which is hereby incorporated by reference in its entirety. In some embodiments, the lipid film can hydrated with water, the hydrated lipid film and payload diluted in buffer, and mechanically mixed by pipetting and/or vortexing to form a liposome population. Other methods for formulation that can be used are sonication and microfluidization. Advantageously, the lipids are formulated as lipid nanoparticles using microfluidic mixing as described, for example, in Roces et al., Pharmaceutics, 12:1095 (2020). Suitable microfluidic mixing devices are commercially available from, for example, Precision Nanosystems (Vancouver, BC). Typically, microfluidic mixing combines two fluid streams, one containing the payload(s) and one containing the ionizable lipid of structure I and optionally other components, such as one or more helper lipids, one or more stabilizing agents and one or more peptides as described herein.

In some embodiments, preparation of the lipoplex formulations for introducing a payload to immune cells may comprise the step of contacting the at least one ionizable lipid of structure I with one or more helper lipids and one or more stabilizing agents before or at the same time as contacting the payload with the at least one ionizable lipid of structure I to form lipoplexes encapsulating the payload. The preparation methods also optionally comprise forming the lipid formulation into lipoplexes prior to contact with the payload. In some embodiments, the lipoplexes are formed by microfluidization, extrusion or other means known in the art.

In some embodiments, methods for preparing a population of lipid complex formulations containing a payload, such as a nucleic acid molecule, comprise: (a) transferring to a mixing container an aqueous solution comprising a buffer and the payload molecule; optionally adding other components such as stabilizing agent(s), peptide(s) or ligand(s) as described herein, (b) injecting a lipid solution comprising the ionizable lipid and optionally other lipid components as described herein into the aqueous solution, wherein the injecting comprises extrusion, in-line mixing, microfluidic mixing, evaporation, or vortexing; and (c) producing the population of lipid formulations complexed with a payload for delivery to an immune cells.

In some embodiments, method for preparing lipoplex formulations containing a payload molecule are provided, which include (a) transferring to a mixing container an aqueous solution comprising a buffer and the payload molecule; (b) injecting a lipid solution comprising an ionizable lipid having structure I and at least one helper lipid into the aqueous solution, wherein the injecting comprises extrusion, in-line mixing, microfluidic mixing, evaporation, or vortexing; and (c) producing the population of lipid formulations complexed with a payload molecule. In some embodiments, methods for preparing lipoplex formulations containing a payload molecule are provided, which include (a) transferring an ionizable lipid having structure I and at least one helper lipid in an aqueous solution to a mixing container comprising a buffer and the payload molecule; (c) mixing the components of step by extrusion, in-line mixing, microfluidic mixing, evaporation, or vortexing; and (d) producing the population of lipid formulations complexed with a payload molecule. In some embodiments, the payload molecule is a nucleic acid molecule.

In some embodiments, the preparation methods produce a population of lipid nanoparticles with the payload molecule encapsulated therein. In other embodiments, the preparation methods produce a population of liposomes with the payload encapsulated therein. In some embodiments, the preparation methods produce a population of lipid nanoparticles with the payload molecule complexed to the exterior of the lipid nanoparticle. In other embodiments, the preparation methods produce a population of liposomes with the payload molecule complexed to the exterior of the liposome. In some of these embodiments, the payload molecule is a nucleic acid molecule.

As described herein, methods are provided for introducing a payload into an immune cell using a lipoplex comprising at least one ionizable lipid having structure I complexed with the payload. Accordingly, provided are methods for transfecting an immune cell with the lipoplex-payload compositions as described herein.

In some embodiments, the cell transfection methods using the lipoplex compositions provided herein are Apolipoprotein E (ApoE) independent. In some embodiments, no ApoE is present in the payload-lipoplex formulation or the transfection reaction. In some embodiments, no ApoE is present in the culture medium before and/or after transfection. In some embodiments, the cells are ApoE negative.

In some embodiments, provided herein are methods for expressing a protein in an immune cell, the methods comprising contacting an immune cell with a nucleic acid payload encoding the protein and a lipoplex comprising at least one ionizable lipid having structure I, or pharmaceutically acceptable salts thereof, under conditions that permit expression of the protein.

In some embodiments, provided are methods for expression of a protein involved in genome editing in an immune cell, the methods comprising contacting an immune cell with a nucleic acid payload encoding a protein involved with genome editing and a lipoplex comprising at least one ionizable lipid having structure I, or pharmaceutically acceptable salts thereof, under conditions that permit expression of the protein for genome editing. In some embodiments, provided are methods for delivery of a nucleic acid payload involved in genome editing into an immune cell, the methods comprising contacting an immune cell with a nucleic acid payload involved with genome editing and a lipoplex comprising at least one ionizable lipid having structure I, or pharmaceutically acceptable salts thereof, under conditions that permit delivery of the nucleic acid for genome editing to the interior of the cell. Examples of such proteins and nucleic acids involved in genome editing are described herein.

In some embodiments, provided are methods for genome editing, the methods comprising contacting an immune cell with a payload involved with genome editing and a lipoplex comprising at least one ionizable lipid having structure I, or pharmaceutically acceptable salts thereof, under conditions that permit delivery of the payload for genome editing to the interior of the cell. In some embodiments, expression of the payload in the cell results in genome editing. In some embodiments, the payload involved in genome editing is a nucleic acid which contributes to genome editing.

In some embodiments of the provided methods, contacting of the immune cell with the payload and lipoplex occurs ex vivo, in vitro, or in vivo.

In some embodiments of the provided methods, the immune cell is cultured in vitro or ex vivo for about 1 day to about 20 days following the contacting. In some embodiments of the provided methods, the immune cell is cultured in vitro or ex vivo for about 1 day to about 14 days following the contacting. In some embodiments, the immune cell is in a population of immune cells during the contacting and the population of immune cells is cultured in vitro or ex vivo for a period of time following the contacting, where viability in the population of immune cells remains in excess of 60% for the period of time. In some embodiments, viability in the population of immune cells remains in excess of 70% for the period of time. In some embodiments, viability in the population of immune cells remains in excess of 80% for the period of time. In some embodiments, the period of time in culture following the contacting is cultured for about 1 day to about 20 days.

In some embodiments of the provided methods, the payload-lipoplex is delivered to the immune cell via in vivo administration. For in vivo administration, the pharmaceutical compositions are preferably administered parenterally (e.g., intraarticularly, intravenously, intraperitoneally, subcutaneously, intrathecally, intradermally, intratracheally, intraosseous, intramuscularly or intratumorally). In particular embodiments, the pharmaceutical compositions are administered intravenously, intrathecally, or intraperitoneally by a bolus injection. Other routes of administration include topical (skin, eyes, mucus membranes), oral, pulmonary, intranasal, sublingual, rectal, and vaginal administration.

In aspects, a kit comprising a lipoplex composition for delivering a payload into a immune cell is provided. In embodiments, the kit comprises the at least one ionizable lipid having a structure I compound and at least one helper lipid, and reagents. In some embodiments, the kit further comprises one or more of a stabilizing agent, a fusion agent, an endosomal release agent, a nuclear localization peptide, a cell targeting agent, and an exosome or a material derived or isolated from an exosome. In embodiments, a lipoplex composition in the kit is suitable for delivery (e.g., via local injection) to a subject.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

### EXAMPLES

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

Based on a systematic Design of Experiment (DOE) approach, compositions including at least one exemplary ionizable lipid of structure I and one or more helper lipids were made and complexed with mRNA using previously developed protocols.

Lipoplex compositions examined included lipid nanoparticle complexes formulated with at least one exemplary ionizable lipid of structure I, DOPE, cholesterol, and 1,2-dimyristoyl-rac-glycero-3-methoxypolyethylene glycol-2000 (DMG-PEG 2000). The lipids were weighed, dissolved and the RNA payload was encapsulated in lipid complexes using microfluidic instrumentation or reverse evaporation and mechanically mixed by pipetting and/or vortexing.

In exemplary formulations analyzed, the ionizable lipid of structure I varied from about 10% to about 60% molar ratio, DOPE varied from about 20% to about 60% molar ratio, cholesterol varied from about 20% to about 60% molar ratio, and DMG-PEG2000 ranged from about 0.5% to about 5.0% molar ratio. When combined with the mRNA, N/P ratios of the lipoplex-mRNA composition varied from 2 to 50. In some formulations, DSPC was used instead of DOPE.

Performance and transfection efficiency of the lipoplex formulations in primary T cells included analysis of payload delivery and expression of the payload encoded protein, and post-transfection cell viability. This was compared to lipoplexes having other ionizable/cationic lipids.

Primary human peripheral blood mononuclear cells were thawed from liquid nitrogen storage and activated with the Dynabeads™ Human T-Activator CD3/CD28 (Thermo Fisher Scientific) (3:1 beads:cell) in CTS™ OpTimizer™ T-Cell Expansion medium (Thermo Fisher Scientific) supplemented with 2% human serum and with 200 U ml-1 IL-2 (Thermo Fisher Scientific) at a ratio of 3:1 beads:cell. The final cell density for activation was 1×10⁶/ml for 3 days activation and 1.5×10⁶/ml for 2 days activation.

The activated T cells were de-beaded and pelleted. The cells were resuspended in the T cell expansion medium without human serum and seeded into wells of a 96-well plate at 200,000 cells/well. The cells were transfected with the lipoplex formulations containing firefly luciferase-encoding mRNA at either 100 ng RNA/well or 300 ng RNA/well. The cells were incubated at 37° C. at 5% CO₂ for 48 hours. At about 16 hours post-transfection, 2% human serum was added back to the cells. The Firefly Luciferase Glow Assay Kit (Pierce, Thermo Scientific) was used determine luciferase activity in the transfected cells. For this, the cells were lysed and cell lysate was added to D-luciferin to measure luciferase activity according to manufacturer instructions. Plates were read on a luminometer plate reader to determined luminescence. To measure cell viability of the transfected cell cultures, PrestoBlue™ HS Cell Viability Reagent (Thermo Fisher Scientific) was used according to manufacturer instructions and the plates were read on a Varioskan LUX plate reader to determine fluorescence intensity. Viability was calculated based on untransfected cells.

Exemplary transfection results are shown in FIGS. 1A-1B and 2A-2C. Lipoplex compositions LP1, LP2 and LP3 are three different formulations containing Compound 32 (as provided herein), DOPE, cholesterol and DMG-PEG2000. As shown, these exemplary lipoplex compositions transfected T cells with high efficiency (FIG. 1A) while also maintaining cell viability (FIG. 1B).

Lipoplex composition LP4 is a formulation containing Compound 43 (as provided herein), DOPE, cholesterol and DMG-PEG2000. The LP1 and LP4 lipoplex formulations containing a GFP-encoding mRNA were used to transfect activated T cells at either 100 ng RNA/well or 150 ng/RNA/well as described above. After 48 hours post-transfection, the plates were read on a Perkin-Elmer fluorescent plate reader to determined GFP intensity. Similar to LP1, this lipoplex composition also transfected activated T cells with high efficiency (FIG. 2A and FIG. 2C) while also maintaining high cell viability (FIG. 2B). In comparison, transfection of activated primary T cells with a lipoplex formulation containing the cationic lipid DMRIE, DOPE, cholesterol and DMG-PEG2000 resulted in an average RLU of 1595 and cell viability of 69% for 100 ng luciferase RNA and an average RLU of 4538 and cell viability of 34% for 300 ng luciferase RNA.

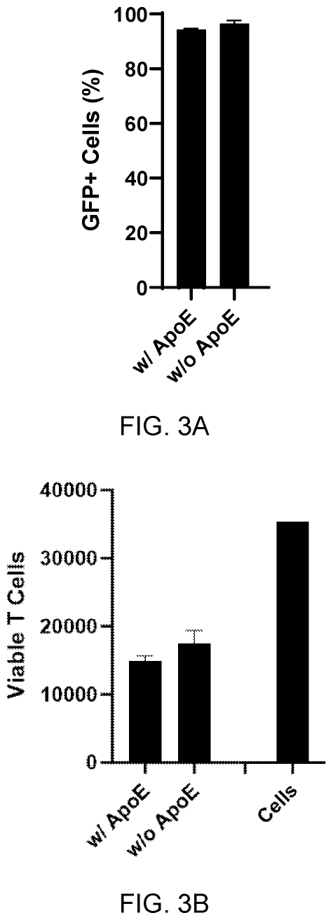

Cell transfection using the lipoplex compositions provided herein is ApoE independent. LP1 lipoplex formulation containing a GFP-encoding mRNA was used to transfect activated T cells at 100 ng RNA/well either with or without 1 μg/ml ApoE4 in the culture medium. After 48 hours post-transfection, the plates were read on a fluorescent plate reader to determine GFP intensity. As shown in FIGS. 3A-3B, use of the LP1 lipoplex composition resulted in greater than 95% transfection efficiency for activated T cells without ApoE4 while also maintaining cell viability. The ApoE independence of the lipoplex compositions provided herein contrasts with ApoE-dependent lipid-based transfection systems.

The results show that the lipid compounds described herein provided effective transfection efficiency, as measured by GFP expression, in activated primary T cells.

### Example 2

TCR α/β knockout was performed using CRISPR/Cas9 technology delivered via lipoplex compositions provided herein to primary activated human T cells. Lipid nanoparticles were prepared by diluting TCR targeting sgRNA/Cas9 mRNA at a 1:1 weight ratio in 100 mM NaOAc (pH 5.2) at 1 μg/90 μL and adding 15 μL formulated lipids. The mixture was immediately vortexed 3 times at high speed. The LNP preparations were concentrated using Amicon centrifugation unit, typically concentrated about 5-fold.

Human T cells were activated as described above, de-beaded and pelleted. The cells were resuspended in the T cell expansion medium without human serum and seeded into wells at 200,000 cells/well. The cells were transfected with 100-500 ng LNP/well of the LNP formulations containing TCR targeting sgRNA/Cas9 mRNA. The cells were incubated for at 37° C. at 5% CO2 for 48 hours. At one day post-transfection, 2% human serum was added back to the cells. After 2-5 days post-transfection, the TCR α/β knockout frequency was tested by flow cytometry. T cell control (no transfection) and post-transfection TCR expression was characterized by flow cytometry staining with PE conjugated TCR alpha/beta antibody (Thermo Fisher Scientific), and analyzed with an Attune N×T flow cytometer (Thermo Fisher Scientific). All data were analyzed with the Flow Jo_V10 software (Tree Star Inc.).

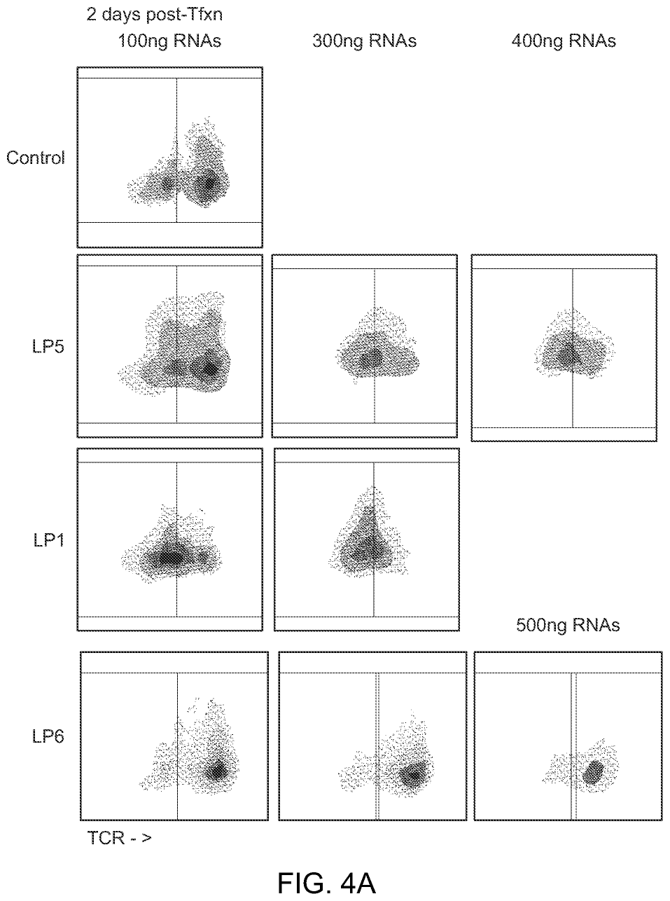

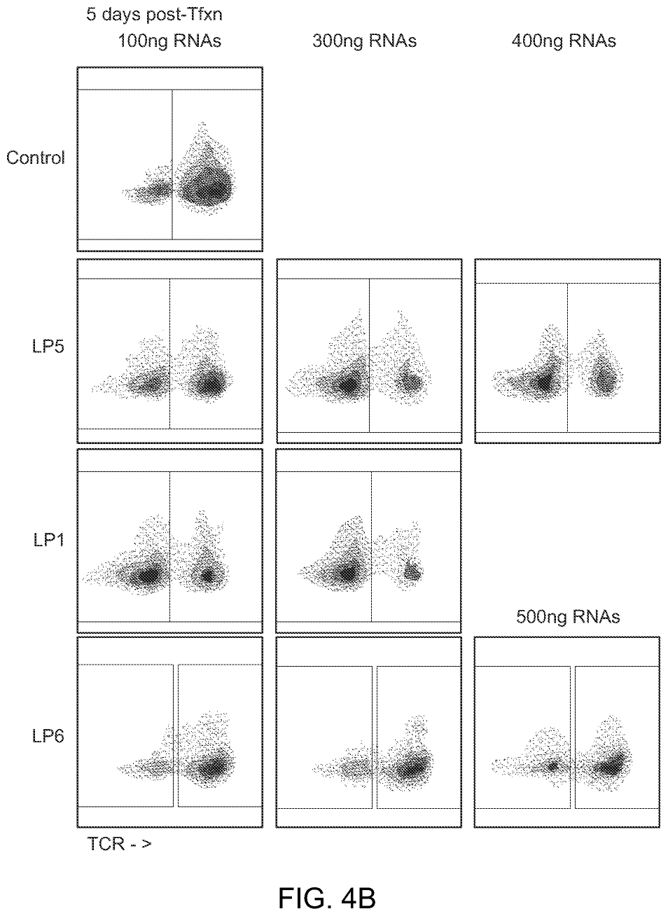

Exemplary gene editing results are shown in FIGS. 4A-4B. Lipoplex composition LP1 is as described above, LP5 contains Compound 32 (as provided herein), DOPE, cholesterol and C16-PEG5000, and LP6 contains Compound 32, DSPC, cholesterol and DMG-PEG2000. The N/P ratios of these three formulations ranges from 10-50. These results demonstrate the high efficiency of gene editing. For example, at day 5 post-transfection with LP1, the final gene editing efficiency reached 85% (FIG. 4B).

### Example 3

Performance and transfection efficiency of the lipoplex formulations in primary mature dendritic cells and primary NK cells included analysis of transfection efficiency as evidenced by expression of the payload encoded protein and post-transfection cell viability. This was compared to lipoplexes having other ionizable/cationic lipids.

Primary human peripheral blood mononuclear cells were thawed from liquid nitrogen storage and human CD14+ monocytes were isolated with Dynabeads™ Untouched™ Human Monocytes Kit (Thermo Fisher Scientific). Human CD14+ monocytes were differentiated for 6 days in CTS AIM-V medium supplemented with 5% ICSR (Immune Cell Serum Replacement, Thermo Fisher Scientific), GM-CSF 0.1 ng/ml, IL-4 0.2 ng/ml. The final cell density was 0.5-1.0×10⁶/ml. At day 6, the medium was replaced with maturation medium, AIM-V medium supplemented with 5% ICSR, 0.1 ng/ml GM-CSF, 0.2 ng/ml IL-4, 10 ng/ml TNF alpha, 10 ng/ml IL-1 beta, 15 ng/ml IL-6, and 1 μg/ml prostaglandin-E2, and the cells were cultured 2 days for maturation.

The mature dendritic cells were dissociated with TrypLE reagent (Thermo Fisher Scientific), pelleted and resuspended in maturation medium and seeded into wells of a 96-well plate at 200,000 cells/well. The cells were transfected with lipoplex formulation LP1 containing GFP-encoding mRNA at either 50 ng RNA/well or 100 ng RNA/well. For comparison, mature dendritic cells were also transfected with MessengerMax transfection reagent (Thermo Fisher Scientific) containing GFP-encoding mRNA at either 50 ng RNA/well or 100 ng RNA/well according to manufacturer's instructions. The cells were incubated at 37° C. at 5% CO₂ for 48 hours. To measure cell viability, SYTOX™ Red Dead Cell Stain or SYTOX™ Green Dead Cell Stain was used as a viability marker (Thermo Fisher Scientific) and samples were analyzed with an Attune N×T flow cytometer (Thermo Fisher Scientific). All data were analyzed with the Flow Jo_V10 software (Tree Star Inc.).

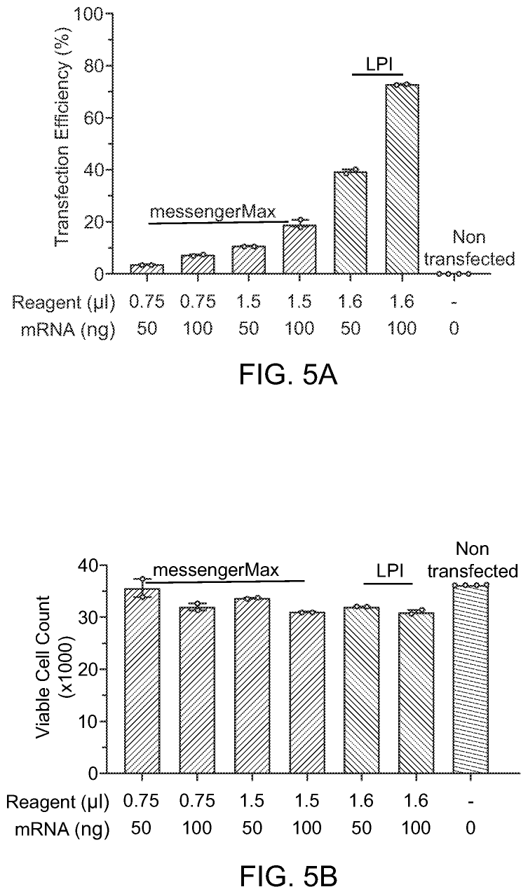

Exemplary transfection results are shown in FIGS. 5A-5B. Lipoplex composition LP1 transfected mature dendritic cells with high efficiency (FIG. 5A) while maintaining cell viability (FIG. 5B). For primary mature dendritic cells, LP1 resulted in about 70% transfection efficiency with 100 ng mRNA payload, significantly exceeding that of commercially available MessengerMax transfection reagent (used according to manufacturer's recommendations).

Primary human peripheral blood mononuclear cells were thawed from liquid nitrogen storage and NK cells were isolated with the Dynabeads™ Human CD3/CD28 (Thermo Fisher Scientific) (10:1 beads:cell) in DPBS −/− supplemented with 2% HSA and 2 mM EDTA, pH7.4 to remove T cells. NK cells were cultured in complete CTS NK-Xpander medium (Thermo Fisher Scientific) supplemented with 5% human serum and with 500 U ml-1 IL-2 (Thermo Fisher Scientific). The final cell density for NK cells was 1.25×10⁵/ml for 5 days. At day 5, the viable cell density (VCD) was checked and NK-Xpander complete medium was added so that the final density of cells was between 4-5×10⁵/ml.

The NK cells were pelleted and resuspended in the CTS NK-Xpander medium without human serum and seeded into wells of a 96-well plate at 200,000 cells/well. The cells were transfected with the lipoplex formulation LP1 containing GFP-encoding mRNA at either 100 ng RNA/well, 150 ng RNA/well or 200 ng RNA/well. The cells were incubated at 37° C. at 5% CO₂ for 48 hours. At about 16 hours post-transfection, 5% human serum was added back to the cells. To measure cell viability, SYTOX™ Red Dead Cell Stain or SYTOX™ Green Dead Cell Stain was used as a viability marker (Thermo Fisher Scientific) and samples were analyzed with an Attune N×T flow cytometer (Thermo Fisher Scientific). All data were analyzed with the Flow Jo_V10 software (Tree Star Inc.).

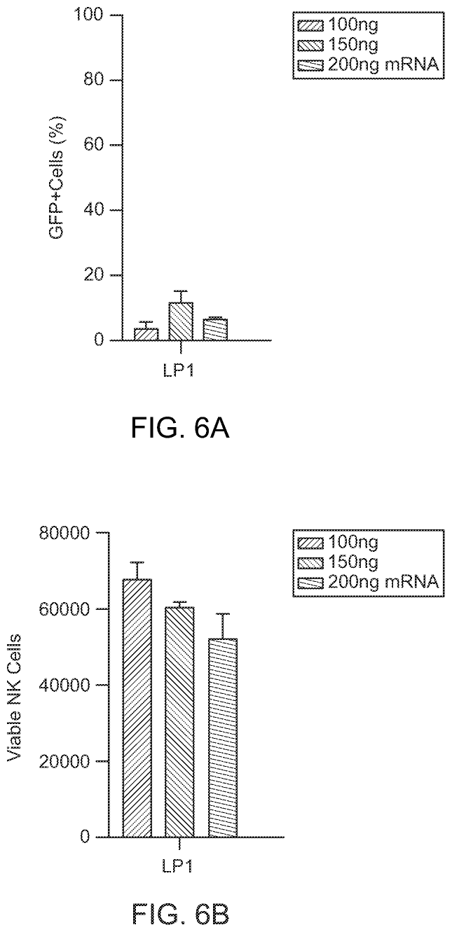

As shown in FIGS. 6A-6B, this lipoplex composition transfected primary NK cells with about 10% transfection efficiency while maintaining high cell viability.

1. A method for introducing a payload into an immune cell, comprising:
contacting an immune cell with a payload and a lipoplex comprising at least one ionizable lipid compound, thereby introducing the payload into the immune cell;
the at least one ionizable lipid compound having the structure I, or pharmaceutically acceptable salts thereof, 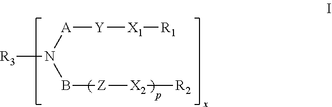
wherein:
each of X₁ and X₂ is a moiety independently selected from the group consisting of O, S, N—W and C—W, wherein W is selected from the group consisting of hydrogen and a C₁-C₂₀ hydrocarbon chain;
each of Y and Z is a moiety independently selected from the group consisting of CH—OH, C═O, C═S, S═O, and SO₂;
R₁, is selected from substituted or unsubstituted, branched or unbranched alkyl or alkenyl groups having between 3 and about 20 carbon atoms and between 0 and about 4 double bonds;
R₂, is selected from H or substituted or unsubstituted, branched or unbranched alkyl or alkenyl groups having between 3 and about 20 carbon atoms and between 0 and about 4 double bonds;
each of A and B is selected from the group consisting of (CR₄R₅)ₙ, (CR₆R₇)ₘ, branched or unbranched alkyl groups having between 3 and about 20 carbon atoms, and
branched or unbranched alkenyl groups having between 3 and about 20 carbon atoms and having between 1 and about 4 double bonds;
each of R₄, R₅, R₆ and R₇ is a moiety independently selected from the group consisting of hydrogen, a cyclic or an acyclic, substituted or unsubstituted, branched or unbranched aliphatic group;
x is an integer independently having the value between 1 and 10, inclusively;
n is an integer independently having the value between 1 and 3, inclusively;
m is an integer independently having the value between 0 and 20, inclusively;
p is an integer independently having the value of 0 or 1; and
R₃ is selected from 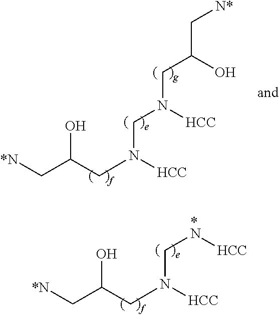
wherein “HCC” symbolizes a straight or branched alkyl, alkenyl or alkynyl hydrocarbon chain having up to about 20 carbon atoms; and each N* indicates the nitrogen atom N which is explicitly present in the above general structure I to which -A-Y—X₁—R₁, and —B—(Z—X₂)ₚ—R₂ are attached; and wherein each g, e and f is independently an integer between 1 and 6.

2. The method of claim 1, wherein the at least one ionizable lipid compound has the structure II, or pharmaceutically acceptable salts thereof, 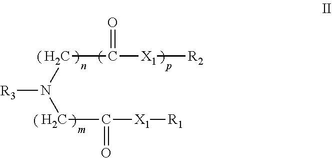
wherein:
each X₁ is independently selected from O or NH;
n is 0, 1, 2, or 3;
m is 1, 2, or 3;
p is 0 or 1, and when n=p=0, R₂ is H.

3-6. (canceled)

7. The method of claim 1, wherein the at least one ionizable lipid is selected from the group consisting of compounds 1-43.

8. The method of claim 1, wherein the immune cell is a T cell, a B cell, natural killer (NK) cell, a dendritic cell, or macrophage.

9-11. (canceled)

12. The method of claim 1, wherein the payload comprises at least one nucleic acid.

13. The method of claim 12, wherein the at least one nucleic acid comprises an RNA molecule.

14-15. (canceled)

16. The method of claim 12, wherein the nucleic acid encodes a chimeric antigen receptor (CAR).

17. (canceled)

18. The method of claim 13, wherein the RNA molecule encodes a gene editing protein.

19-24. (canceled)

25. The method of claim 1, wherein the at least one ionizable lipid comprises a biodegradable linkage.

26. The method of claim 1, wherein the at least one ionizable lipid in the lipoplex has a protonatable group with a pKa in the range of about 4 to about 8.

27-28. (canceled)

29. The method of claim 1, wherein the lipoplex comprises at least one helper lipid.

30-32. (canceled)

33. The method of claim 1, wherein the lipoplex comprises at least one stabilizing agent is selected from a surfactant, a polymer conjugated lipid, and polyethylene glycol.

34. (canceled)

35. The method of claim 1, wherein the lipoplex further comprises a fusion agent, wherein said agent optionally comprises a polycationic nucleic acid binding moiety.

36-44.

45. The method of claim 1, wherein the lipoplex comprises the at least one ionizable lipid, at least one helper lipid, and at least one stabilizing agent.

46. The method of claim 45, wherein the at least one ionizable lipid is present in the lipoplex at a compositional molar ratio of about 0.10 to about 0.70.

47. The method of claim 45, wherein the at least one helper lipid is present in the lipoplex at a compositional molar ratio of about 0.10 to about 0.90.

48. The method of claim 45, wherein the at least one stabilizing agent is present in the lipoplex at a compositional molar ratio of about 0.005 to about 0.10.

49-58. (canceled)

59. A lipoplex for delivering a payload into an immune cell comprising:
the at least one ionizable lipid compound having the structure I, or pharmaceutically acceptable salts thereof, 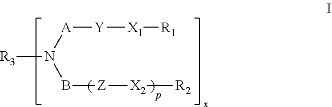
wherein:
each of X₁ and X₂ is a moiety independently selected from the group consisting of O, S, N—W and C—W, wherein W is selected from the group consisting of hydrogen and a C₁-C₂₀ hydrocarbon chain;
each of Y and Z is a moiety independently selected from the group consisting of CH—OH, C═O, C═S, S═O, and SO₂;
R₁, is selected from substituted or unsubstituted, branched or unbranched alkyl or alkenyl groups having between 3 and about 20 carbon atoms and between 0 and about 4 double bonds;
R₂, is selected from H or substituted or unsubstituted, branched or unbranched alkyl or alkenyl groups having between 3 and about 20 carbon atoms and between 0 and about 4 double bonds;
each of A and B is selected from the group consisting of (CR₄R₅)ₙ, (CR₆R₇)ₘ, branched or unbranched alkyl groups having between 3 and about 20 carbon atoms, and
branched or unbranched alkenyl groups having between 3 and about 20 carbon atoms and having between 1 and about 4 double bonds;
each of R₄, R₅, R₆ and R₇ is a moiety independently selected from the group consisting of hydrogen, a cyclic or an acyclic, substituted or unsubstituted, branched or unbranched aliphatic group;
x is an integer independently having the value between 1 and 10, inclusively;
n is an integer independently having the value between 1 and 3, inclusively;
m is an integer independently having the value between 0 and 20, inclusively;
p is an integer independently having the value of 0 or 1; and
R₃ is selected from 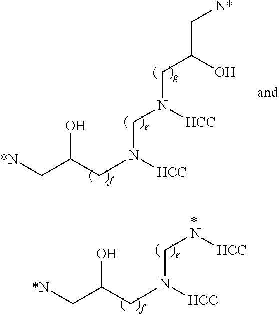
wherein “HCC” symbolizes a straight or branched alkyl, alkenyl or alkynyl hydrocarbon chain having up to about 20 carbon atoms; and each N* indicates the nitrogen atom N which is explicitly present in the above general structure I to which -A-Y—X₁—R₁, and —B—(Z—X₂)ₚ—R₂ are attached; and
wherein each g, e and f is independently an integer between 1 and 6.

60. The lipoplex of claim 59, further comprising a payload.

61. The lipoplex of claim 59, wherein the lipoplex comprises the at least one ionizable lipid, at least one helper lipid, and at least one stabilizing agent.


SEQUENCE LISTING (584 sequences)

SEQ ID NO: 1 - protein, 15 aa, synthetic construct
  REGION 1..15: note = Description of Artificial Sequence: Synthetic peptide
GYSTPPKKKRKVEDP

SEQ ID NO: 2 - protein, 12 aa, synthetic construct
  REGION 1..12: note = Description of Artificial Sequence: Synthetic peptide
GYSTPPKTRRRP

SEQ ID NO: 3 - protein, 10 aa, synthetic construct
  REGION 1..10: note = Description of Artificial Sequence: Synthetic peptide
GYSTPGRKKR

SEQ ID NO: 4 - protein, 13 aa, synthetic construct
  REGION 1..13: note = Description of Artificial Sequence: Synthetic peptide
GYSTPRRNRRRRW

SEQ ID NO: 5 - protein, 15 aa, synthetic construct
  REGION 1..15: note = Description of Artificial Sequence: Synthetic peptide
PDEVKRKKKPPTSYG

SEQ ID NO: 6 - protein, 12 aa, synthetic construct
  REGION 1..12: note = Description of Artificial Sequence: Synthetic peptide
PRRRTKPPTSYG

SEQ ID NO: 7 - protein, 10 aa, synthetic construct
  REGION 1..10: note = Description of Artificial Sequence: Synthetic peptide
RKKRGPTSYG

SEQ ID NO: 8 - protein, 13 aa, synthetic construct
  REGION 1..13: note = Description of Artificial Sequence: Synthetic peptide
WRRRRNRRPTSYG

SEQ ID NO: 9 - protein, 17 aa, synthetic construct
  REGION 1..17: note = Description of Artificial Sequence: Synthetic peptide
GYGPPKKKRKVEAPYKA

SEQ ID NO: 10 - protein, 9 aa, synthetic construct
  REGION 1..9: note = Description of Artificial Sequence: Synthetic peptide
PAAKRVKLD

SEQ ID NO: 11 - protein, 11 aa, synthetic construct
  REGION 1..11: note = Description of Artificial Sequence: Synthetic peptide
RQRRNELKRSP

SEQ ID NO: 12 - protein, 16 aa, synthetic construct
  REGION 1..16: note = Description of Artificial Sequence: Synthetic peptide
KRPAATKKAGQAKKKK

SEQ ID NO: 13 - protein, 24 aa, synthetic construct
  REGION 1..24: note = Description of Artificial Sequence: Synthetic peptide
VRKKRKTEEESPLKDKDAKKSKQE

SEQ ID NO: 14 - protein, 24 aa, synthetic construct
  REGION 1..24: note = Description of Artificial Sequence: Synthetic peptide
RLRRDAGGRGGVYEHLGGAPRRRK

SEQ ID NO: 15 - protein, 19 aa, synthetic construct
  REGION 1..19: note = Description of Artificial Sequence: Synthetic peptide
KRKGDEVDGVDECAKKSKK

SEQ ID NO: 16 - protein, 38 aa, synthetic construct
  REGION 1..38: note = Description of Artificial Sequence: Synthetic polypeptide
NQSSNFGPMKGGNFGGRSSGPYGGGGQYFAKPRNQGGY

SEQ ID NO: 17 - protein, 27 aa, synthetic construct
  REGION 1..27: note = Description of Artificial Sequence: Synthetic peptide
GGKRTADGSEFESPKKARKVEAYPKAW

SEQ ID NO: 18 - protein, 27 aa, synthetic construct
  REGION 1..27: note = Description of Artificial Sequence: Synthetic peptide
GGKRTADGSEFESPKKKRAVEAYPKAW

SEQ ID NO: 19 - protein, 27 aa, synthetic construct
  REGION 1..27: note = Description of Artificial Sequence: Synthetic peptide
GGKRTADGSEFESPKKKAKVEAYPKAW

SEQ ID NO: 20 - protein, 28 aa, synthetic construct
  REGION 1..28: note = Description of Artificial Sequence: Synthetic peptide
GGKRTADGSEFESPKKKRKVEAPYKAWK

SEQ ID NO: 21 - protein, 26 aa, synthetic construct
  REGION 1..26: note = Description of Artificial Sequence: Synthetic peptide
GGKRTADGSEFESPKKKRKVEYKAWK

SEQ ID NO: 22 - protein, 20 aa, synthetic construct
  REGION 1..20: note = Description of Artificial Sequence: Synthetic peptide
GYGPAAKRVKLDEAYPKAWK

SEQ ID NO: 23 - protein, 29 aa, synthetic construct
  REGION 1..29: note = Description of Artificial Sequence: Synthetic peptide
GGKRTADGSEFEPAAKRVKLDEAYPKAWK

SEQ ID NO: 24 - protein, 24 aa, synthetic construct
  REGION 1..24: note = Description of Artificial Sequence: Synthetic peptide
GTGPKKKRKVGGGGYGPKKKRLVG

SEQ ID NO: 25 - protein, 25 aa, synthetic construct
  REGION 1..25: note = Description of Artificial Sequence: Synthetic peptide
KRPAATKKAGQAKKKKLEAYPKAWK

SEQ ID NO: 26 - protein, 16 aa, synthetic construct
  REGION 1..16: note = Description of Artificial Sequence: Synthetic peptide
ATKGTKRSYEQMETGE

SEQ ID NO: 27 - protein, 12 aa, synthetic construct
  REGION 1..12: note = Description of Artificial Sequence: Synthetic peptide
GKWERKPIRCAS

SEQ ID NO: 28 - protein, 29 aa, synthetic construct
  REGION 1..29: note = Description of Artificial Sequence: Synthetic peptide
GYGKRTADSQHSTPPKKKRKVEAPYKAWK

SEQ ID NO: 29 - protein, 26 aa, synthetic construct
  REGION 1..26: note = Description of Artificial Sequence: Synthetic peptide
KRTADSQHSTPPKKKRKVEAPYKAWK

SEQ ID NO: 30 - protein, 46 aa, synthetic construct
  REGION 1..46: note = Description of Artificial Sequence: Synthetic polypeptide
GYGPPKKKRKVEAPYKAWKWAKYPAMRRAHHRRRRASHRRRTTTGT

SEQ ID NO: 31 - protein, 43 aa, synthetic construct
  REGION 1..43: note = Description of Artificial Sequence: Synthetic polypeptide
GYGPPKKKRKVEAPYKAWKRGARRYSKMKRRRRRVARRHRRRP

SEQ ID NO: 32 - protein, 23 aa, synthetic construct
  REGION 1..23: note = Description of Artificial Sequence: Synthetic peptide
FWGYGYGPPKKKRKVEAPYKAWK

SEQ ID NO: 33 - protein, 28 aa, synthetic construct
  REGION 1..28: note = Description of Artificial Sequence: Synthetic peptide
GKPSSDDEATADSQHSTPPKKKERKVED

SEQ ID NO: 34 - protein, 21 aa, synthetic construct
  REGION 1..21: note = Description of Artificial Sequence: Synthetic peptide
GKPTADDQHSTPPKKKRKVED

SEQ ID NO: 35 - protein, 27 aa, synthetic construct
  REGION 1..27: note = Description of Artificial Sequence: Synthetic peptide
GGKRTADGSEFESPKKARKVEAYPKAK

SEQ ID NO: 36 - protein, 18 aa, synthetic construct
  REGION 1..18: note = Description of Artificial Sequence: Synthetic peptide
EKIRLRPGRKKRYRLKHL

SEQ ID NO: 37 - protein, 18 aa, synthetic construct
  REGION 1..18: note = Description of Artificial Sequence: Synthetic peptide
PEGTRQARRNRRRRWRKR

SEQ ID NO: 38 - protein, 18 aa, synthetic construct
  REGION 1..18: note = Description of Artificial Sequence: Synthetic peptide
PEGTRQPRRNRRRRWRKR

SEQ ID NO: 39 - protein, 26 aa, synthetic construct
  REGION 1..26: note = Description of Artificial Sequence: Synthetic peptide
GVKRSYGAARGDDRRRPNVVAPYKAW

SEQ ID NO: 40 - protein, 26 aa, synthetic construct
  REGION 1..26: note = Description of Artificial Sequence: Synthetic peptide
KSVPNRTRTYIKLKRLRFKGAPYKAW

SEQ ID NO: 41 - protein, 27 aa, synthetic construct
  REGION 1..27: note = Description of Artificial Sequence: Synthetic peptide
EMRRRREEEGLQLRKQKREEQLFKRRN

SEQ ID NO: 42 - protein, 14 aa, synthetic construct
  REGION 1..14: note = Description of Artificial Sequence: Synthetic peptide
FEAALAEALAEALA

SEQ ID NO: 43 - protein, 12 aa, synthetic construct
  REGION 1..12: note = Description of Artificial Sequence: Synthetic peptide
  REGION 1..12: note = N-term Ac
  REGION 1..12: note = C-term NHCH3
LARLLPRLLARL

SEQ ID NO: 44 - protein, 20 aa, synthetic construct
  REGION 1..20: note = Description of Artificial Sequence: Synthetic peptide
GLLEELLELLEELWEELLEG

SEQ ID NO: 45 - protein, 19 aa, synthetic construct
  REGION 1..19: note = Description of Artificial Sequence: Synthetic peptide
GWEGLIEGIEGGWEGLIEG

SEQ ID NO: 46 - protein, 20 aa, synthetic construct
  REGION 1..20: note = Description of Artificial Sequence: Synthetic peptide
GLFEALAEFIEGGWEGLIEG

SEQ ID NO: 47 - protein, 20 aa, synthetic construct
  REGION 1..20: note = Description of Artificial Sequence: Synthetic peptide
GLFEALLELLESLWELLLEA

SEQ ID NO: 48 - protein, 22 aa, synthetic construct
  REGION 1..22: note = Description of Artificial Sequence: Synthetic peptide
GGYCLEKWMIVASELKCFGNTA

SEQ ID NO: 49 - protein, 23 aa, synthetic construct
  REGION 1..23: note = Description of Artificial Sequence: Synthetic peptide
GGYCLTRWMLIEAELKCFGNTAV

SEQ ID NO: 50 - protein, 30 aa, synthetic construct
  REGION 1..30: note = Description of Artificial Sequence: Synthetic polypeptide
WEAALAEALAEALAEHLAEALAEALEALAA

SEQ ID NO: 51 - protein, 23 aa, synthetic construct
  REGION 1..23: note = Description of Artificial Sequence: Synthetic peptide
GLFGAIAGFIENGWEGMIDGWYG

SEQ ID NO: 52 - protein, 26 aa, synthetic construct
  REGION 1..26: note = Description of Artificial Sequence: Synthetic peptide
GIGAVLKVLTTGLPALISWIKRKRQQ

SEQ ID NO: 53 - protein, 13 aa, synthetic construct
  REGION 1..13: note = Description of Artificial Sequence: Synthetic peptide
GRKKRRQRRRPPQ

SEQ ID NO: 54 - protein, 16 aa, synthetic construct
  REGION 1..16: note = Description of Artificial Sequence: Synthetic peptide
RQIKIWFQNRRMKWKK

SEQ ID NO: 55 - protein, 27 aa, synthetic construct
  REGION 1..27: note = Description of Artificial Sequence: Synthetic peptide
GWTLNSAGYLLGKINLKALAALAKKIL

SEQ ID NO: 56 - protein, 30 aa, synthetic construct
  REGION 1..30: note = Description of Artificial Sequence: Synthetic polypeptide
WEAKLAKALAKALAKHLAKALAKALKACEA

SEQ ID NO: 57 - protein, 20 aa, synthetic construct
  REGION 1..20: note = Description of Artificial Sequence: Synthetic peptide
GLFKALLKLLKSLWKLLLKA

SEQ ID NO: 58 - protein, 20 aa, synthetic construct
  REGION 1..20: note = Description of Artificial Sequence: Synthetic peptide
GLFRALLRLLRSLWRLLLRA

SEQ ID NO: 59 - protein, 20 aa, synthetic construct
  REGION 1..20: note = Description of Artificial Sequence: Synthetic peptide
GLFEALLELLESLYELLLEA

SEQ ID NO: 60 - protein, 12 aa, synthetic construct
  REGION 1..12: note = Description of Artificial Sequence: Synthetic peptide
GLFEALEELWEA

SEQ ID NO: 61 - protein, 10 aa, synthetic construct
  REGION 1..10: note = Description of Artificial Sequence: Synthetic peptide
GLFLLEEWLE

SEQ ID NO: 62 - protein, 11 aa, synthetic construct
  REGION 1..11: note = Description of Artificial Sequence: Synthetic peptide
GLFLLEEWLEK

SEQ ID NO: 63 - protein, 21 aa, synthetic construct
  REGION 1..21: note = Description of Artificial Sequence: Synthetic peptide
GLFEALLELLESLWELLLEAK

SEQ ID NO: 64 - protein, 11 aa, synthetic construct
  REGION 1..11: note = Description of Artificial Sequence: Synthetic peptide
  REGION 1..11: note = N-term succinyl
GLFKLLEEWLE

SEQ ID NO: 65 - protein, 12 aa, synthetic construct
  REGION 1..12: note = Description of Artificial Sequence: Synthetic peptide
  REGION 1..12: note = N-term succinyl
GLFKLLEEWLEK

SEQ ID NO: 66 - protein, 20 aa, synthetic construct
  REGION 1..20: note = Description of Artificial Sequence: Synthetic peptide
GLFEAIAEFIEGGWEGLIEG

SEQ ID NO: 67 - protein, 20 aa, synthetic construct
  REGION 1..20: note = Description of Artificial Sequence: Synthetic peptide
GLFKAIAKFIKGGWKGLIKG

SEQ ID NO: 68 - protein, 17 aa, synthetic construct
  REGION 1..17: note = Description of Artificial Sequence: Synthetic peptide
IRFKKTKLIASIAMALC

SEQ ID NO: 69 - protein, 30 aa, synthetic construct
  REGION 1..30: note = Description of Artificial Sequence: Synthetic polypeptide
ALAGTIIAGASLTFQVLDKVLEELGKVSRK

SEQ ID NO: 70 - protein, 23 aa, synthetic construct
  REGION 1..23: note = Description of Artificial Sequence: Synthetic peptide
GLFEAIEGFIENGWEGMIDGWYG

SEQ ID NO: 71 - protein, 17 aa, synthetic construct
  REGION 1..17: note = Description of Artificial Sequence: Synthetic peptide
GYICRRARGDNPDDRCT

SEQ ID NO: 72 - protein, 22 aa, synthetic construct
  REGION 1..22: note = Description of Artificial Sequence: Synthetic peptide
GLFEAIAEFIEGGWEGLIEGCA

SEQ ID NO: 73 - protein, 24 aa, synthetic construct
  REGION 1..24: note = Description of Artificial Sequence: Synthetic peptide
GLFHAIAHFIHGGWHGLIHGWWYG

SEQ ID NO: 74 - protein, 30 aa, synthetic construct
  REGION 1..30: note = Description of Artificial Sequence: Synthetic polypeptide
RRRQRRKKRGGDIMGEWGNEIFGAIAGFLG

SEQ ID NO: 75 - protein, 22 aa, synthetic construct
  REGION 1..22: note = Description of Artificial Sequence: Synthetic peptide
GLFEAIADFIENGWEGMIDGGG

SEQ ID NO: 76 - protein, 31 aa, synthetic construct
  REGION 1..31: note = Description of Artificial Sequence: Synthetic polypeptide
ALAGTIIAGASLTFQVLDKVLEELGKVSRKK

SEQ ID NO: 77 - protein, 15 aa, synthetic construct
  REGION 1..15: note = Description of Artificial Sequence: Synthetic peptide
IRFKKTKLIASIAMA

SEQ ID NO: 78 - protein, 18 aa, synthetic construct
  REGION 1..18: note = Description of Artificial Sequence: Synthetic peptide
GLWHLLLHLWRRLLRLLR

SEQ ID NO: 79 - protein, 21 aa, synthetic construct
  REGION 1..21: note = Description of Artificial Sequence: Synthetic peptide
KKIMLLLMTLLLVSLPLAQEQ

SEQ ID NO: 80 - protein, 23 aa, synthetic construct
  REGION 1..23: note = Description of Artificial Sequence: Synthetic peptide
GLFEALLELLESLWELLLEAWYG

SEQ ID NO: 81 - protein, 18 aa, synthetic construct
  REGION 1..18: note = Description of Artificial Sequence: Synthetic peptide
RLLRLLLRLWRRLLRLLR

SEQ ID NO: 82 - protein, 26 aa, synthetic construct
  REGION 1..26: note = Description of Artificial Sequence: Synthetic peptide
LLELELLELELLLELELLELELLLEL

SEQ ID NO: 83 - protein, 28 aa, synthetic construct
  REGION 1..28: note = Description of Artificial Sequence: Synthetic peptide
GLFEALLELLESLWELLLEARRRRRRRR

SEQ ID NO: 84 - protein, 26 aa, synthetic construct
  REGION 1..26: note = Description of Artificial Sequence: Synthetic peptide
GLFEALLELLESLWELLLEARRRRRR

SEQ ID NO: 85 - protein, 28 aa, synthetic construct
  REGION 1..28: note = Description of Artificial Sequence: Synthetic peptide
GLFEALLELLESLWELLLEAKKKKKKKK

SEQ ID NO: 86 - protein, 26 aa, synthetic construct
  REGION 1..26: note = Description of Artificial Sequence: Synthetic peptide
GLFEALLELLESLWELLLEAKKKKKK

SEQ ID NO: 87 - protein, 22 aa, synthetic construct
  REGION 1..22: note = Description of Artificial Sequence: Synthetic peptide
GLFEALLELLESLWELLLEAKK

SEQ ID NO: 88 - protein, 24 aa, synthetic construct
  REGION 1..24: note = Description of Artificial Sequence: Synthetic peptide
GLFEALLELLESLWELLLEAKKKK

SEQ ID NO: 89 - protein, 22 aa, synthetic construct
  REGION 1..22: note = Description of Artificial Sequence: Synthetic peptide
GLFEALLELLESLWELLLEAEE

SEQ ID NO: 90 - protein, 24 aa, synthetic construct
  REGION 1..24: note = Description of Artificial Sequence: Synthetic peptide
GLFEALLELLESLWELLLEAEEEE

SEQ ID NO: 91 - protein, 26 aa, synthetic construct
  REGION 1..26: note = Description of Artificial Sequence: Synthetic peptide
GLFEALLELLESLWELLLEAEEEEEE

SEQ ID NO: 92 - protein, 18 aa, synthetic construct
  REGION 1..18: note = Description of Artificial Sequence: Synthetic peptide
GLFEALLELLESLWELLL

SEQ ID NO: 93 - protein, 36 aa, synthetic construct
  REGION 1..36: note = Description of Artificial Sequence: Synthetic polypeptide
PLSSIFSRIGDPRGARRYAKMKRRRRRVARRHRRRP

SEQ ID NO: 94 - protein, 13 aa, synthetic construct
  REGION 1..13: note = Description of Artificial Sequence: Synthetic peptide
GPFHYFQFLFPPV

SEQ ID NO: 95 - protein, 13 aa, synthetic construct
  REGION 1..13: note = Description of Artificial Sequence: Synthetic peptide
GSSSWWQRWWPPW

SEQ ID NO: 96 - protein, 9 aa, synthetic construct
  REGION 1..9: note = Description of Artificial Sequence: Synthetic peptide
RRRQRRKKR

SEQ ID NO: 97 - protein, 4 aa, synthetic construct
  REGION 1..4: note = Description of Artificial Sequence: Synthetic peptide
KKKK

SEQ ID NO: 98 - protein, 6 aa, synthetic construct
  REGION 1..6: note = Description of Artificial Sequence: Synthetic peptide
KKKKKK

SEQ ID NO: 99 - protein, 8 aa, synthetic construct
  REGION 1..8: note = Description of Artificial Sequence: Synthetic peptide
KKKKKKKK

SEQ ID NO: 100 - protein, 10 aa, synthetic construct
  REGION 1..10: note = Description of Artificial Sequence: Synthetic peptide
KKKKKKKKKK

SEQ ID NO: 101 - protein, 12 aa, synthetic construct
  REGION 1..12: note = Description of Artificial Sequence: Synthetic peptide
KKKKKKKKKKKK

SEQ ID NO: 102 - protein, 16 aa, synthetic construct
  REGION 1..16: note = Description of Artificial Sequence: Synthetic peptide
KKKKKKKKKKKKKKKK

SEQ ID NO: 103 - protein, 20 aa, synthetic construct
  REGION 1..20: note = Description of Artificial Sequence: Synthetic peptide
KKKKKKKKKKKKKKKKKKKK

SEQ ID NO: 104 - protein, 24 aa, synthetic construct
  REGION 1..24: note = Description of Artificial Sequence: Synthetic peptide
KKKKKKKKKKKKKKKKKKKKKKKK

SEQ ID NO: 105 - protein, 4 aa, synthetic construct
  REGION 1..4: note = Description of Artificial Sequence: Synthetic peptide
RRRR

SEQ ID NO: 106 - protein, 6 aa, synthetic construct
  REGION 1..6: note = Description of Artificial Sequence: Synthetic peptide
RRRRRR

SEQ ID NO: 107 - protein, 8 aa, synthetic construct
  REGION 1..8: note = Description of Artificial Sequence: Synthetic peptide
RRRRRRRR

SEQ ID NO: 108 - protein, 10 aa, synthetic construct
  REGION 1..10: note = Description of Artificial Sequence: Synthetic peptide
RRRRRRRRRR

SEQ ID NO: 109 - protein, 12 aa, synthetic construct
  REGION 1..12: note = Description of Artificial Sequence: Synthetic peptide
RRRRRRRRRRRR

SEQ ID NO: 110 - protein, 16 aa, synthetic construct
  REGION 1..16: note = Description of Artificial Sequence: Synthetic peptide
RRRRRRRRRRRRRRRR

SEQ ID NO: 111 - protein, 20 aa, synthetic construct
  REGION 1..20: note = Description of Artificial Sequence: Synthetic peptide
RRRRRRRRRRRRRRRRRRRR

SEQ ID NO: 112 - protein, 24 aa, synthetic construct
  REGION 1..24: note = Description of Artificial Sequence: Synthetic peptide
RRRRRRRRRRRRRRRRRRRRRRRR

SEQ ID NO: 113 - skipped

SEQ ID NO: 114 - protein, 25 aa, synthetic construct
  REGION 1..25: note = Description of Artificial Sequence: Synthetic peptide
KKKKKKKKWKGGGGACYGLPHLFCG

SEQ ID NO: 115 - protein, 13 aa, synthetic construct
  REGION 1..13: note = Description of Artificial Sequence: Synthetic peptide
YKAKKKKKKKKWK

SEQ ID NO: 116 - protein, 18 aa, synthetic construct
  REGION 1..18: note = Description of Artificial Sequence: Synthetic peptide
KTPKKAKKPKTPKKAKKP

SEQ ID NO: 117 - protein, 29 aa, synthetic construct
  REGION 1..29: note = Description of Artificial Sequence: Synthetic peptide
KKAKKPAATRKSSKNPKKPKTVKPKKVAK

SEQ ID NO: 118 - protein, 24 aa, synthetic construct
  REGION 1..24: note = Description of Artificial Sequence: Synthetic peptide
RGARRYSKMKRRRRRVARRHRRRP

SEQ ID NO: 119 - protein, 22 aa, synthetic construct
  REGION 1..22: note = Description of Artificial Sequence: Synthetic peptide
TRQARRNRRRRWRERQRGSGSG

SEQ ID NO: 120 - protein, 29 aa, synthetic construct
  REGION 1..29: note = Description of Artificial Sequence: Synthetic peptide
KRPRGRPKGSKKNWRRRKRRASRRSPRRR

SEQ ID NO: 121 - protein, 34 aa, synthetic construct
  REGION 1..34: note = Description of Artificial Sequence: Synthetic polypeptide
KRGRGRPRKQPPKEPSEVPTPKRPRGRPKGSKNK

SEQ ID NO: 122 - protein, 33 aa, synthetic construct
  REGION 1..33: note = Description of Artificial Sequence: Synthetic polypeptide
KEKYEKDIAAYRAKGKPAAKKGVVKAEKSKKKK

SEQ ID NO: 123 - protein, 15 aa, synthetic construct
  REGION 1..15: note = Description of Artificial Sequence: Synthetic peptide
YKAKKKKKKKKKKWK

SEQ ID NO: 124 - protein, 10 aa, synthetic construct
  REGION 1..10: note = Description of Artificial Sequence: Synthetic peptide
KKKKKKKGGC

SEQ ID NO: 125 - protein, 13 aa, synthetic construct
  REGION 1..13: note = Description of Artificial Sequence: Synthetic peptide
YRARRRRRRRRWR

SEQ ID NO: 126 - protein, 15 aa, synthetic construct
  REGION 1..15: note = Description of Artificial Sequence: Synthetic peptide
YRARRRRRRRRRRWR

SEQ ID NO: 127 - protein, 42 aa, synthetic construct
  REGION 1..42: note = Description of Artificial Sequence: Synthetic polypeptide
KGDPKKPRGKMSSYAFFVQTCREEHKKKHPDASVNFSEFSKK

SEQ ID NO: 128 - protein, 16 aa, synthetic construct
  REGION 1..16: note = Description of Artificial Sequence: Synthetic peptide
KKQLKKQLKKQLKQWK

SEQ ID NO: 129 - protein, 16 aa, synthetic construct
  REGION 1..16: note = Description of Artificial Sequence: Synthetic peptide
KKSPKKSPKKSPKKSK

SEQ ID NO: 130 - protein, 13 aa, synthetic construct
  REGION 1..13: note = Description of Artificial Sequence: Synthetic peptide
KLSKLEKKSKLEK

SEQ ID NO: 131 - protein, 20 aa, synthetic construct
  REGION 1..20: note = Description of Artificial Sequence: Synthetic peptide
KLSKLEKKLSKLEKKSKLEK

SEQ ID NO: 132 - protein, 15 aa, synthetic construct
  REGION 1..15: note = Description of Artificial Sequence: Synthetic peptide
KSLKKSLKKSLKKSK

SEQ ID NO: 133 - protein, 22 aa, synthetic construct
  REGION 1..22: note = Description of Artificial Sequence: Synthetic peptide
KIRRRGKNKVAARTCRQRRTDR

SEQ ID NO: 134 - protein, 22 aa, synthetic construct
  REGION 1..22: note = Description of Artificial Sequence: Synthetic peptide
KIRRRGKNKVAAQNCRKRKLET

SEQ ID NO: 135 - protein, 24 aa, synthetic construct
  REGION 1..24: note = Description of Artificial Sequence: Synthetic peptide
KRRIRREKNKMAAAKCRNRRRELT

SEQ ID NO: 136 - protein, 12 aa, synthetic construct
  REGION 1..12: note = Description of Artificial Sequence: Synthetic peptide
KDRSNLLERHTR

SEQ ID NO: 137 - protein, 16 aa, synthetic construct
  REGION 1..16: note = Description of Artificial Sequence: Synthetic peptide
KRPAATKKAGQAKKKL

SEQ ID NO: 138 - protein, 10 aa, synthetic construct
  REGION 1..10: note = Description of Artificial Sequence: Synthetic peptide
RRRRRREEEE

SEQ ID NO: 139 - protein, 12 aa, synthetic construct
  REGION 1..12: note = Description of Artificial Sequence: Synthetic peptide
RRRRRREEEEEE

SEQ ID NO: 140 - protein, 14 aa, synthetic construct
  REGION 1..14: note = Description of Artificial Sequence: Synthetic peptide
RRRRRREEEEEEEE

SEQ ID NO: 141 - protein, 12 aa, synthetic construct
  REGION 1..12: note = Description of Artificial Sequence: Synthetic peptide
RRRRRRRREEEE

SEQ ID NO: 142 - protein, 14 aa, synthetic construct
  REGION 1..14: note = Description of Artificial Sequence: Synthetic peptide
RRRRRRRREEEEEE

SEQ ID NO: 143 - protein, 16 aa, synthetic construct
  REGION 1..16: note = Description of Artificial Sequence: Synthetic peptide
RRRRRRRREEEEEEEE

SEQ ID NO: 144 - protein, 16 aa, synthetic construct
  REGION 1..16: note = Description of Artificial Sequence: Synthetic peptide
RRRRRRRRRRRREEEE

SEQ ID NO: 145 - protein, 18 aa, synthetic construct
  REGION 1..18: note = Description of Artificial Sequence: Synthetic peptide
RRRRRRRRRRRREEEEEE

SEQ ID NO: 146 - protein, 18 aa, synthetic construct
  REGION 1..18: note = Description of Artificial Sequence: Synthetic peptide
RRRRRRRRRRRREEEEEE

SEQ ID NO: 147 - protein, 8 aa, synthetic construct
  REGION 1..8: note = Description of Artificial Sequence: Synthetic peptide
KLSKLEKK

SEQ ID NO: 148 - protein, 5 aa, synthetic construct
  REGION 1..5: note = Description of Artificial Sequence: Synthetic peptide
SKLEK

SEQ ID NO: 149 - protein, 15 aa, synthetic construct
  REGION 1..15: note = Description of Artificial Sequence: Synthetic peptide
KLSKLEKKLSKLEKK

SEQ ID NO: 150 - protein, 15 aa, synthetic construct
  REGION 1..15: note = Description of Artificial Sequence: Synthetic peptide
PKKKRKVGGGRGDSP

SEQ ID NO: 151 - protein, 9 aa, synthetic construct
  REGION 1..9: note = Description of Artificial Sequence: Synthetic peptide
LPHKSMPCG

SEQ ID NO: 152 - protein, 12 aa, synthetic construct
  REGION 1..12: note = Description of Artificial Sequence: Synthetic peptide
GACLQHKSMPCG

SEQ ID NO: 153 - protein, 9 aa, synthetic construct
  REGION 1..9: note = Description of Artificial Sequence: Synthetic peptide
YGLPHLFCG

SEQ ID NO: 154 - protein, 9 aa, synthetic construct
  REGION 1..9: note = Description of Artificial Sequence: Synthetic peptide
SERSMNFCG

SEQ ID NO: 155 - protein, 15 aa, synthetic construct
  REGION 1..15: note = Description of Artificial Sequence: Synthetic peptide
DHYSLYEDLERGTDK

SEQ ID NO: 156 - protein, 16 aa, synthetic construct
  REGION 1..16: note = Description of Artificial Sequence: Synthetic peptide
ISLPRTSGAQRASTTR

SEQ ID NO: 157 - protein, 17 aa, synthetic construct
  REGION 1..17: note = Description of Artificial Sequence: Synthetic peptide
EKLQTKYGLPHKVEFCG

SEQ ID NO: 158 - protein, 12 aa, synthetic construct
  REGION 1..12: note = Description of Artificial Sequence: Synthetic peptide
TRISESQAKPGD

SEQ ID NO: 159 - protein, 6 aa, synthetic construct
  REGION 1..6: note = Description of Artificial Sequence: Synthetic peptide
LVFFDY

SEQ ID NO: 160 - protein, 21 aa, synthetic construct
  REGION 1..21: note = Description of Artificial Sequence: Synthetic peptide
WGGNGPTTFDCSGYTKYVFAK

SEQ ID NO: 161 - protein, 15 aa, synthetic construct
  REGION 1..15: note = Description of Artificial Sequence: Synthetic peptide
INIGTTGWGDHYSLY

SEQ ID NO: 162 - protein, 6 aa, synthetic construct
  REGION 1..6: note = Description of Artificial Sequence: Synthetic peptide
YDNIHG

SEQ ID NO: 163 - protein, 13 aa, synthetic construct
  REGION 1..13: note = Description of Artificial Sequence: Synthetic peptide
AGWGKFLVGFGRV

SEQ ID NO: 164 - protein, 7 aa, synthetic construct
  REGION 1..7: note = Description of Artificial Sequence: Synthetic peptide
SIGYPLP

SEQ ID NO: 165 - protein, 8 aa, synthetic construct
  REGION 1..8: note = Description of Artificial Sequence: Synthetic peptide
TTHWGFTL

SEQ ID NO: 166 - protein, 12 aa, synthetic construct
  REGION 1..12: note = Description of Artificial Sequence: Synthetic peptide
HLQIQPYPQISG

SEQ ID NO: 167 - protein, 9 aa, synthetic construct
  REGION 1..9: note = Description of Artificial Sequence: Synthetic peptide
KLNIVSVNG

SEQ ID NO: 168 - skipped

SEQ ID NO: 169 - protein, 11 aa, synthetic construct
  REGION 1..11: note = Description of Artificial Sequence: Synthetic peptide
DNRIRLQAKAA

SEQ ID NO: 170 - protein, 10 aa, synthetic construct
  REGION 1..10: note = Description of Artificial Sequence: Synthetic peptide
KIKMVISWKG

SEQ ID NO: 171 - protein, 12 aa, synthetic construct
  REGION 1..12: note = Description of Artificial Sequence: Synthetic peptide
LPWYSYLYAVSA

SEQ ID NO: 172 - protein, 12 aa, synthetic construct
  REGION 1..12: note = Description of Artificial Sequence: Synthetic peptide
WNLPWYYSVSPT

SEQ ID NO: 173 - skipped

SEQ ID NO: 174 - protein, 9 aa, synthetic construct
  REGION 1..9: note = Description of Artificial Sequence: Synthetic peptide
PWYYSVSPT

SEQ ID NO: 175 - protein, 14 aa, synthetic construct
  REGION 1..14: note = Description of Artificial Sequence: Synthetic peptide
SSWESYKSGGGTRL

SEQ ID NO: 176 - protein, 18 aa, synthetic construct
  REGION 1..18: note = Description of Artificial Sequence: Synthetic peptide
RDWSSQHPGRCNGETHLK

SEQ ID NO: 177 - protein, 7 aa, synthetic construct
  REGION 1..7: note = Description of Artificial Sequence: Synthetic peptide
SLPTLTL

SEQ ID NO: 178 - protein, 21 aa, synthetic construct
  REGION 1..21: note = Description of Artificial Sequence: Synthetic peptide
VICTGGDYSFALPVGQWPVMT

SEQ ID NO: 179 - protein, 23 aa, synthetic construct
  REGION 1..23: note = Description of Artificial Sequence: Synthetic peptide
DKPSYQFGGHNSVDFEEDTLPKV

SEQ ID NO: 180 - protein, 26 aa, synthetic construct
  REGION 1..26: note = Description of Artificial Sequence: Synthetic peptide
RARRRKRASATQLYQTCKASGTCPPD

SEQ ID NO: 181 - protein, 18 aa, synthetic construct
  REGION 1..18: note = Description of Artificial Sequence: Synthetic peptide
SGDYSFALPVGQWPWMTG

SEQ ID NO: 182 - protein, 17 aa, synthetic construct
  REGION 1..17: note = Description of Artificial Sequence: Synthetic peptide
CTGGDYSFALPVGQWPW

SEQ ID NO: 183 - protein, 15 aa, synthetic construct
  REGION 1..15: note = Description of Artificial Sequence: Synthetic peptide
FYYDYDFFFDYWGQG

SEQ ID NO: 184 - protein, 15 aa, synthetic construct
  REGION 1..15: note = Description of Artificial Sequence: Synthetic peptide
HLRRLRRRLLREAEG

SEQ ID NO: 185 - protein, 17 aa, synthetic construct
  REGION 1..17: note = Description of Artificial Sequence: Synthetic peptide
DYYCAAWDDSLNGYSVF

SEQ ID NO: 186 - protein, 15 aa, synthetic construct
  REGION 1..15: note = Description of Artificial Sequence: Synthetic peptide
YYCLQSMEDPYTFGG

SEQ ID NO: 187 - protein, 20 aa, synthetic construct
  REGION 1..20: note = Description of Artificial Sequence: Synthetic peptide
YYCARSDGNYGYYYALDYDY

SEQ ID NO: 188 - protein, 12 aa, synthetic construct
  REGION 1..12: note = Description of Artificial Sequence: Synthetic peptide
AARSPSYYRYDY

SEQ ID NO: 189 - protein, 9 aa, synthetic construct
  REGION 1..9: note = Description of Artificial Sequence: Synthetic peptide
GPYYAMDYD

SEQ ID NO: 190 - protein, 22 aa, synthetic construct
  REGION 1..22: note = Description of Artificial Sequence: Synthetic peptide
YYCQQRSSYPYTEGGAYPKAWK

SEQ ID NO: 191 - protein, 18 aa, synthetic construct
  REGION 1..18: note = Description of Artificial Sequence: Synthetic peptide
YYCQRYDSDWSFGQGTKL

SEQ ID NO: 192 - protein, 18 aa, synthetic construct
  REGION 1..18: note = Description of Artificial Sequence: Synthetic peptide
YYCARSGYYAMDYWGQGT

SEQ ID NO: 193 - protein, 13 aa, synthetic construct
  REGION 1..13: note = Description of Artificial Sequence: Synthetic peptide
RVRRGACRGDCLG

SEQ ID NO: 194 - protein, 13 aa, synthetic construct
  REGION 1..13: note = Description of Artificial Sequence: Synthetic peptide
RVRRGACRYDCLG

SEQ ID NO: 195 - protein, 23 aa, synthetic construct
  REGION 1..23: note = Description of Artificial Sequence: Synthetic peptide
YYCAKGTHWGFWSGYFDYWGQGT

SEQ ID NO: 196 - protein, 17 aa, synthetic construct
  REGION 1..17: note = Description of Artificial Sequence: Synthetic peptide
GRENYHGCTTHWGFTLC

SEQ ID NO: 197 - protein, 17 aa, synthetic construct
  REGION 1..17: note = Description of Artificial Sequence: Synthetic peptide
VQATQSNQHTPRGGGSK

SEQ ID NO: 198 - protein, 7 aa, synthetic construct
  REGION 1..7: note = Description of Artificial Sequence: Synthetic peptide
DPRAPGS

SEQ ID NO: 199 - protein, 15 aa, synthetic construct
  REGION 1..15: note = Description of Artificial Sequence: Synthetic peptide
YYCQQRSSYPYTFGG

SEQ ID NO: 200 - protein, 21 aa, synthetic construct
  REGION 1..21: note = Description of Artificial Sequence: Synthetic peptide
AARSPSYYRYDYGPYYAMDYD

SEQ ID NO: 201 - protein, 18 aa, synthetic construct
  REGION 1..18: note = Description of Artificial Sequence: Synthetic peptide
GPKLTGILISILSLFVES

SEQ ID NO: 202 - protein, 11 aa, synthetic construct
  REGION 1..11: note = Description of Artificial Sequence: Synthetic peptide
KYILRWRPKNS

SEQ ID NO: 203 - protein, 5 aa, synthetic construct
  REGION 1..5: note = Description of Artificial Sequence: Synthetic peptide
IKVAV

SEQ ID NO: 204 - protein, 20 aa, synthetic construct
  REGION 1..20: note = Description of Artificial Sequence: Synthetic peptide
WTPPRAQITGYRLTVGLTRR

SEQ ID NO: 205 - protein, 12 aa, synthetic construct
  REGION 1..12: note = Description of Artificial Sequence: Synthetic peptide
AASIKVAVSADR

SEQ ID NO: 206 - protein, 6 aa, synthetic construct
  REGION 1..6: note = Description of Artificial Sequence: Synthetic peptide
KLDAPT

SEQ ID NO: 207 - protein, 12 aa, synthetic construct
  REGION 1..12: note = Description of Artificial Sequence: Synthetic peptide
NRWHSIYITRFG

SEQ ID NO: 208 - protein, 5 aa, synthetic construct
  REGION 1..5: note = Description of Artificial Sequence: Synthetic peptide
PHSRN

SEQ ID NO: 209 - protein, 12 aa, synthetic construct
  REGION 1..12: note = Description of Artificial Sequence: Synthetic peptide
SSFHFDGSGYAM

SEQ ID NO: 210 - protein, 4 aa, synthetic construct
  REGION 1..4: note = Description of Artificial Sequence: Synthetic peptide
RGDS

SEQ ID NO: 211 - protein, 6 aa, synthetic construct
  REGION 1..6: note = Description of Artificial Sequence: Synthetic peptide
IAFQRN

SEQ ID NO: 212 - protein, 6 aa, synthetic construct
  REGION 1..6: note = Description of Artificial Sequence: Synthetic peptide
GRGDSP

SEQ ID NO: 213 - protein, 11 aa, synthetic construct
  REGION 1..11: note = Description of Artificial Sequence: Synthetic peptide
TWYKIAFQRRK

SEQ ID NO: 214 - protein, 7 aa, synthetic construct
  REGION 1..7: note = Description of Artificial Sequence: Synthetic peptide
EDGIHEL

SEQ ID NO: 215 - protein, 12 aa, synthetic construct
  REGION 1..12: note = Description of Artificial Sequence: Synthetic peptide
SLVRNRRVITIQ

SEQ ID NO: 216 - protein, 16 aa, synthetic construct
  REGION 1..16: note = Description of Artificial Sequence: Synthetic peptide
YRVRVTPKEKTGPMKE

SEQ ID NO: 217 - protein, 7 aa, synthetic construct
  REGION 1..7: note = Description of Artificial Sequence: Synthetic peptide
LQVQLSR

SEQ ID NO: 218 - protein, 9 aa, synthetic construct
  REGION 1..9: note = Description of Artificial Sequence: Synthetic peptide
SPPRRARVT

SEQ ID NO: 219 - protein, 12 aa, synthetic construct
  REGION 1..12: note = Description of Artificial Sequence: Synthetic peptide
RKRLQVQLSIRT

SEQ ID NO: 220 - protein, 9 aa, synthetic construct
  REGION 1..9: note = Description of Artificial Sequence: Synthetic peptide
ATETTITIS

SEQ ID NO: 221 - protein, 12 aa, synthetic construct
  REGION 1..12: note = Description of Artificial Sequence: Synthetic peptide
NAPFPKLSWTIQ

SEQ ID NO: 222 - protein, 31 aa, synthetic construct
  REGION 1..31: note = Description of Artificial Sequence: Synthetic polypeptide
VSPPRRARVTDATETTITISWRTKTETITGG

SEQ ID NO: 223 - protein, 12 aa, synthetic construct
  REGION 1..12: note = Description of Artificial Sequence: Synthetic peptide
WTIQTTVDRGLL

SEQ ID NO: 224 - protein, 11 aa, synthetic construct
  REGION 1..11: note = Description of Artificial Sequence: Synthetic peptide
KPDVRSYTITG

SEQ ID NO: 225 - protein, 13 aa, synthetic construct
  REGION 1..13: note = Description of Artificial Sequence: Synthetic peptide
DTINNGRDHMILI

SEQ ID NO: 226 - protein, 12 aa, synthetic construct
  REGION 1..12: note = Description of Artificial Sequence: Synthetic peptide
ANGQTPIQRYIK

SEQ ID NO: 227 - protein, 13 aa, synthetic construct
  REGION 1..13: note = Description of Artificial Sequence: Synthetic peptide
MILISIGKSQKRM

SEQ ID NO: 228 - protein, 30 aa, synthetic construct
  REGION 1..30: note = Description of Artificial Sequence: Synthetic polypeptide
PRARITGYIIKYEKPGSPPREVVPRPRPGV

SEQ ID NO: 229 - protein, 12 aa, synthetic construct
  REGION 1..12: note = Description of Artificial Sequence: Synthetic peptide
PPFLMLLKGSTR

SEQ ID NO: 230 - protein, 8 aa, synthetic construct
  REGION 1..8: note = Description of Artificial Sequence: Synthetic peptide
WQPPRARI

SEQ ID NO: 231 - protein, 13 aa, synthetic construct
  REGION 1..13: note = Description of Artificial Sequence: Synthetic peptide
NQRLASFSNAQQS

SEQ ID NO: 232 - protein, 19 aa, synthetic construct
  REGION 1..19: note = Description of Artificial Sequence: Synthetic peptide
WQPPRARITGYIIKYEKPG

SEQ ID NO: 233 - protein, 17 aa, synthetic construct
  REGION 1..17: note = Description of Artificial Sequence: Synthetic peptide
ISNVFVQRMSQSPEVLD

SEQ ID NO: 234 - protein, 19 aa, synthetic construct
  REGION 1..19: note = Description of Artificial Sequence: Synthetic peptide
YEKPGSPPREVVPRPRPGV

SEQ ID NO: 235 - protein, 13 aa, synthetic construct
  REGION 1..13: note = Description of Artificial Sequence: Synthetic peptide
KARSFNVNQLLQD

SEQ ID NO: 236 - protein, 15 aa, synthetic construct
  REGION 1..15: note = Description of Artificial Sequence: Synthetic peptide
KNNQKSEPLIGRKKT

SEQ ID NO: 237 - protein, 12 aa, synthetic construct
  REGION 1..12: note = Description of Artificial Sequence: Synthetic peptide
KNSFMALYLSKG

SEQ ID NO: 238 - protein, 8 aa, synthetic construct
  REGION 1..8: note = Description of Artificial Sequence: Synthetic peptide
EILDVPST

SEQ ID NO: 239 - protein, 19 aa, synthetic construct
  REGION 1..19: note = Description of Artificial Sequence: Synthetic peptide
KNSFMALYLSKGRLVFALG

SEQ ID NO: 240 - protein, 5 aa, synthetic construct
  REGION 1..5: note = Description of Artificial Sequence: Synthetic peptide
IDAPS

SEQ ID NO: 241 - protein, 20 aa, synthetic construct
  REGION 1..20: note = Description of Artificial Sequence: Synthetic peptide
RDSFVALYLSEGHVIFAGLG

SEQ ID NO: 242 - protein, 15 aa, synthetic construct
  REGION 1..15: note = Description of Artificial Sequence: Synthetic peptide
VVIDASTAIDAPSNL

SEQ ID NO: 243 - protein, 12 aa, synthetic construct
  REGION 1..12: note = Description of Artificial Sequence: Synthetic peptide
KPRLQFSLDIQT

SEQ ID NO: 244 - protein, 5 aa, synthetic construct
  REGION 1..5: note = Description of Artificial Sequence: Synthetic peptide
LDVPS

SEQ ID NO: 245 - protein, 12 aa, synthetic construct
  REGION 1..12: note = Description of Artificial Sequence: Synthetic peptide
DGQWHSVTVSIK

SEQ ID NO: 246 - protein, 4 aa, synthetic construct
  REGION 1..4: note = Description of Artificial Sequence: Synthetic peptide
REDV

SEQ ID NO: 247 - protein, 12 aa, synthetic construct
  REGION 1..12: note = Description of Artificial Sequence: Synthetic peptide
FVLYLGSKNAKK

SEQ ID NO: 248 - protein, 10 aa, synthetic construct
  REGION 1..10: note = Description of Artificial Sequence: Synthetic peptide
PHSRNRGDSP

SEQ ID NO: 249 - protein, 12 aa, synthetic construct
  REGION 1..12: note = Description of Artificial Sequence: Synthetic peptide
LAIKNDNLVYVY

SEQ ID NO: 250 - protein, 13 aa, synthetic construct
  REGION 1..13: note = Description of Artificial Sequence: Synthetic peptide
LWVTVRSQQRGLF

SEQ ID NO: 251 - protein, 12 aa, synthetic construct
  REGION 1..12: note = Description of Artificial Sequence: Synthetic peptide
AYFSIVKIERVG

SEQ ID NO: 252 - protein, 13 aa, synthetic construct
  REGION 1..13: note = Description of Artificial Sequence: Synthetic peptide
GTNNWWQSPSIQN

SEQ ID NO: 253 - protein, 12 aa, synthetic construct
  REGION 1..12: note = Description of Artificial Sequence: Synthetic peptide
DVISLYNFKHIY

SEQ ID NO: 254 - protein, 13 aa, synthetic construct
  REGION 1..13: note = Description of Artificial Sequence: Synthetic peptide
WVTVTLDLRQVFQ

SEQ ID NO: 255 - protein, 12 aa, synthetic construct
  REGION 1..12: note = Description of Artificial Sequence: Synthetic peptide
FFDGSSYAVVRD

SEQ ID NO: 256 - protein, 13 aa, synthetic construct
  REGION 1..13: note = Description of Artificial Sequence: Synthetic peptide
RQVFQVAYIIIKA

SEQ ID NO: 257 - protein, 14 aa, synthetic construct
  REGION 1..14: note = Description of Artificial Sequence: Synthetic peptide
LHVFYDFGFGFSNG

SEQ ID NO: 258 - protein, 14 aa, synthetic construct
  REGION 1..14: note = Description of Artificial Sequence: Synthetic peptide
LTRYKITPRRGPPT

SEQ ID NO: 259 - protein, 20 aa, synthetic construct
  REGION 1..20: note = Description of Artificial Sequence: Synthetic peptide
LKKAQINDAKYREISIIYHN

SEQ ID NO: 260 - protein, 12 aa, synthetic construct
  REGION 1..12: note = Description of Artificial Sequence: Synthetic peptide
LLEFTSARYIRL

SEQ ID NO: 261 - protein, 12 aa, synthetic construct
  REGION 1..12: note = Description of Artificial Sequence: Synthetic peptide
RAYFNGQSFIAS

SEQ ID NO: 262 - protein, 12 aa, synthetic construct
  REGION 1..12: note = Description of Artificial Sequence: Synthetic peptide
YIRLRLQRIRTL

SEQ ID NO: 263 - protein, 12 aa, synthetic construct
  REGION 1..12: note = Description of Artificial Sequence: Synthetic peptide
SRLRGKNPTKGK

SEQ ID NO: 264 - protein, 12 aa, synthetic construct
  REGION 1..12: note = Description of Artificial Sequence: Synthetic peptide
RRYYYSIKDISV

SEQ ID NO: 265 - protein, 12 aa, synthetic construct
  REGION 1..12: note = Description of Artificial Sequence: Synthetic peptide
LHKKGKNSSKPK

SEQ ID NO: 266 - protein, 12 aa, synthetic construct
  REGION 1..12: note = Description of Artificial Sequence: Synthetic peptide
SINNTAVNQRLT

SEQ ID NO: 267 - protein, 12 aa, synthetic construct
  REGION 1..12: note = Description of Artificial Sequence: Synthetic peptide
RLKTRSSHGMIF

SEQ ID NO: 268 - protein, 12 aa, synthetic construct
  REGION 1..12: note = Description of Artificial Sequence: Synthetic peptide
GGFLKYTVSYDI

SEQ ID NO: 269 - protein, 12 aa, synthetic construct
  REGION 1..12: note = Description of Artificial Sequence: Synthetic peptide
GEKSQFSIRLKT

SEQ ID NO: 270 - protein, 12 aa, synthetic construct
  REGION 1..12: note = Description of Artificial Sequence: Synthetic peptide
RDQLMTVLANVT

SEQ ID NO: 271 - protein, 12 aa, synthetic construct
  REGION 1..12: note = Description of Artificial Sequence: Synthetic peptide
TLFLAHGRLVFM

SEQ ID NO: 272 - protein, 12 aa, synthetic construct
  REGION 1..12: note = Description of Artificial Sequence: Synthetic peptide
ANVTHLLIRANY

SEQ ID NO: 273 - protein, 12 aa, synthetic construct
  REGION 1..12: note = Description of Artificial Sequence: Synthetic peptide
LVFMFNVGHKKL

SEQ ID NO: 274 - protein, 13 aa, synthetic construct
  REGION 1..13: note = Description of Artificial Sequence: Synthetic peptide
AGTFALRGDNPQG

SEQ ID NO: 275 - protein, 20 aa, synthetic construct
  REGION 1..20: note = Description of Artificial Sequence: Synthetic peptide
TLFLAHGRLVFMFNVGHKKL

SEQ ID NO: 276 - protein, 12 aa, synthetic construct
  REGION 1..12: note = Description of Artificial Sequence: Synthetic peptide
VLIKGGRARKHV

SEQ ID NO: 277 - protein, 19 aa, synthetic construct
  REGION 1..19: note = Description of Artificial Sequence: Synthetic peptide
DFMTLFLAHGRLVFMGNVG

SEQ ID NO: 278 - protein, 11 aa, synthetic construct
  REGION 1..11: note = Description of Artificial Sequence: Synthetic peptide
LSNIDYLIKAS

SEQ ID NO: 279 - protein, 12 aa, synthetic construct
  REGION 1..12: note = Description of Artificial Sequence: Synthetic peptide
HKKLKIRSQEKY

SEQ ID NO: 280 - protein, 12 aa, synthetic construct
  REGION 1..12: note = Description of Artificial Sequence: Synthetic peptide
LQQSRIANISME

SEQ ID NO: 281 - protein, 12 aa, synthetic construct
  REGION 1..12: note = Description of Artificial Sequence: Synthetic peptide
GAAWKIKGPIYL

SEQ ID NO: 282 - protein, 12 aa, synthetic construct
  REGION 1..12: note = Description of Artificial Sequence: Synthetic peptide
NLLLLLVKANLK

SEQ ID NO: 283 - protein, 12 aa, synthetic construct
  REGION 1..12: note = Description of Artificial Sequence: Synthetic peptide
VIRDSNVVQLDV

SEQ ID NO: 284 - protein, 12 aa, synthetic construct
  REGION 1..12: note = Description of Artificial Sequence: Synthetic peptide
HRDELLLWARKI

SEQ ID NO: 285 - protein, 12 aa, synthetic construct
  REGION 1..12: note = Description of Artificial Sequence: Synthetic peptide
GLIYYVAHQNQM

SEQ ID NO: 286 - protein, 12 aa, synthetic construct
  REGION 1..12: note = Description of Artificial Sequence: Synthetic peptide
KRRARDLVHRAE

SEQ ID NO: 287 - protein, 19 aa, synthetic construct
  REGION 1..19: note = Description of Artificial Sequence: Synthetic peptide
DYATLQLQEGRLHFMFDLG

SEQ ID NO: 288 - protein, 12 aa, synthetic construct
  REGION 1..12: note = Description of Artificial Sequence: Synthetic peptide
SQFQESVDNITK

SEQ ID NO: 289 - protein, 12 aa, synthetic construct
  REGION 1..12: note = Description of Artificial Sequence: Synthetic peptide
KKGSYNNIVVHV

SEQ ID NO: 290 - protein, 12 aa, synthetic construct
  REGION 1..12: note = Description of Artificial Sequence: Synthetic peptide
PGGMREKGRKAR

SEQ ID NO: 291 - protein, 12 aa, synthetic construct
  REGION 1..12: note = Description of Artificial Sequence: Synthetic peptide
ADNLLFYLGSAK

SEQ ID NO: 292 - protein, 12 aa, synthetic construct
  REGION 1..12: note = Description of Artificial Sequence: Synthetic peptide
MEMQANLLLDRL

SEQ ID NO: 293 - protein, 12 aa, synthetic construct
  REGION 1..12: note = Description of Artificial Sequence: Synthetic peptide
GSAKFIDFLAIE

SEQ ID NO: 294 - protein, 11 aa, synthetic construct
  REGION 1..11: note = Description of Artificial Sequence: Synthetic peptide
LSEIKLLISAR

SEQ ID NO: 295 - protein, 12 aa, synthetic construct
  REGION 1..12: note = Description of Artificial Sequence: Synthetic peptide
KVSFLWWVGSGV

SEQ ID NO: 296 - protein, 15 aa, synthetic construct
  REGION 1..15: note = Description of Artificial Sequence: Synthetic peptide
RDFTKATNIRLRFLR

SEQ ID NO: 297 - protein, 12 aa, synthetic construct
  REGION 1..12: note = Description of Artificial Sequence: Synthetic peptide
SYWYRIEASRTG

SEQ ID NO: 298 - protein, 12 aa, synthetic construct
  REGION 1..12: note = Description of Artificial Sequence: Synthetic peptide
ISTVMFKFRTFS

SEQ ID NO: 299 - protein, 12 aa, synthetic construct
  REGION 1..12: note = Description of Artificial Sequence: Synthetic peptide
YFDGTGFAKAVG

SEQ ID NO: 300 - protein, 13 aa, synthetic construct
  REGION 1..13: note = Description of Artificial Sequence: Synthetic peptide
KQANISIVDIDSN

SEQ ID NO: 301 - protein, 12 aa, synthetic construct
  REGION 1..12: note = Description of Artificial Sequence: Synthetic peptide
NGQWHKVTAKKI

SEQ ID NO: 302 - protein, 12 aa, synthetic construct
  REGION 1..12: note = Description of Artificial Sequence: Synthetic peptide
FSTRNESGIILL

SEQ ID NO: 303 - protein, 12 aa, synthetic construct
  REGION 1..12: note = Description of Artificial Sequence: Synthetic peptide
AKKIKNRLELVV

SEQ ID NO: 304 - protein, 12 aa, synthetic construct
  REGION 1..12: note = Description of Artificial Sequence: Synthetic peptide
RRQTTQAYYAIF

SEQ ID NO: 305 - protein, 14 aa, synthetic construct
  REGION 1..14: note = Description of Artificial Sequence: Synthetic peptide
GFPGGLNQFGLTTN

SEQ ID NO: 306 - protein, 12 aa, synthetic construct
  REGION 1..12: note = Description of Artificial Sequence: Synthetic peptide
YAIFLNKGRLEV

SEQ ID NO: 307 - protein, 13 aa, synthetic construct
  REGION 1..13: note = Description of Artificial Sequence: Synthetic peptide
NQFGLTTNIRFRG

SEQ ID NO: 308 - protein, 12 aa, synthetic construct
  REGION 1..12: note = Description of Artificial Sequence: Synthetic peptide
KNRLTIELEVRT

SEQ ID NO: 309 - protein, 13 aa, synthetic construct
  REGION 1..13: note = Description of Artificial Sequence: Synthetic peptide
IRSLKLTKGTGKP

SEQ ID NO: 310 - protein, 12 aa, synthetic construct
  REGION 1..12: note = Description of Artificial Sequence: Synthetic peptide
GLLFYMARINHA

SEQ ID NO: 311 - protein, 13 aa, synthetic construct
  REGION 1..13: note = Description of Artificial Sequence: Synthetic peptide
AKALELRGVQPVS

SEQ ID NO: 312 - protein, 12 aa, synthetic construct
  REGION 1..12: note = Description of Artificial Sequence: Synthetic peptide
VQLRNGFPYFSY

SEQ ID NO: 313 - protein, 13 aa, synthetic construct
  REGION 1..13: note = Description of Artificial Sequence: Synthetic peptide
GQLFHVAYILIKF

SEQ ID NO: 314 - protein, 12 aa, synthetic construct
  REGION 1..12: note = Description of Artificial Sequence: Synthetic peptide
HKIKIVRVKQEG

SEQ ID NO: 315 - protein, 12 aa, synthetic construct
  REGION 1..12: note = Description of Artificial Sequence: Synthetic peptide
NVLSLYNFKTTF

SEQ ID NO: 316 - protein, 19 aa, synthetic construct
  REGION 1..19: note = Description of Artificial Sequence: Synthetic peptide
DFGTVQLRNGFPFFSYDLG

SEQ ID NO: 317 - protein, 13 aa, synthetic construct
  REGION 1..13: note = Description of Artificial Sequence: Synthetic peptide
SQRIYQFAKLNYT

SEQ ID NO: 318 - protein, 12 aa, synthetic construct
  REGION 1..12: note = Description of Artificial Sequence: Synthetic peptide
NIRLRFLRTNTL

SEQ ID NO: 319 - protein, 13 aa, synthetic construct
  REGION 1..13: note = Description of Artificial Sequence: Synthetic peptide
EVNVTLDLGQVFH

SEQ ID NO: 320 - protein, 12 aa, synthetic construct
  REGION 1..12: note = Description of Artificial Sequence: Synthetic peptide
GKNTGDHFVLYM

SEQ ID NO: 321 - protein, 13 aa, synthetic construct
  REGION 1..13: note = Description of Artificial Sequence: Synthetic peptide
GQVFHVAYVLIKF

SEQ ID NO: 322 - protein, 13 aa, synthetic construct
  REGION 1..13: note = Description of Artificial Sequence: Synthetic peptide
VVSLYNFEQTFML

SEQ ID NO: 323 - protein, 21 aa, synthetic construct
  REGION 1..21: note = Description of Artificial Sequence: Synthetic peptide
HQQDLGTAGSCLRKFSTMFLF

SEQ ID NO: 324 - protein, 12 aa, synthetic construct
  REGION 1..12: note = Description of Artificial Sequence: Synthetic peptide
RFDQELRLVSYN

SEQ ID NO: 325 - protein, 24 aa, synthetic construct
  REGION 1..24: note = Description of Artificial Sequence: Synthetic peptide
HQQDLGTAGSCLRKFSTMFLFCNI

SEQ ID NO: 326 - protein, 13 aa, synthetic construct
  REGION 1..13: note = Description of Artificial Sequence: Synthetic peptide
RLVSYSGVLFFLK

SEQ ID NO: 327 - protein, 9 aa, synthetic construct
  REGION 1..9: note = Description of Artificial Sequence: Synthetic peptide
VAEIDGIEL

SEQ ID NO: 328 - protein, 12 aa, synthetic construct
  REGION 1..12: note = Description of Artificial Sequence: Synthetic peptide
NWRHISYITRFG

SEQ ID NO: 329 - protein, 6 aa, synthetic construct
  REGION 1..6: note = Description of Artificial Sequence: Synthetic peptide
GIIFFL

SEQ ID NO: 330 - protein, 12 aa, synthetic construct
  REGION 1..12: note = Description of Artificial Sequence: Synthetic peptide
KRLQVQLRSIRT

SEQ ID NO: 331 - protein, 12 aa, synthetic construct
  REGION 1..12: note = Description of Artificial Sequence: Synthetic peptide
ASKAIQVFLLGG

SEQ ID NO: 332 - protein, 12 aa, synthetic construct
  REGION 1..12: note = Description of Artificial Sequence: Synthetic peptide
TWYKIAFQRNRK

SEQ ID NO: 333 - protein, 12 aa, synthetic construct
  REGION 1..12: note = Description of Artificial Sequence: Synthetic peptide
VLVRVERATVFS

SEQ ID NO: 334 - protein, 12 aa, synthetic construct
  REGION 1..12: note = Description of Artificial Sequence: Synthetic peptide
QVFQVAYIIIKA

SEQ ID NO: 335 - protein, 12 aa, synthetic construct
  REGION 1..12: note = Description of Artificial Sequence: Synthetic peptide
TVFSVDQDNMLE

SEQ ID NO: 336 - protein, 13 aa, synthetic construct
  REGION 1..13: note = Description of Artificial Sequence: Synthetic peptide
GEFYFDLRLKGDK

SEQ ID NO: 337 - protein, 12 aa, synthetic construct
  REGION 1..12: note = Description of Artificial Sequence: Synthetic peptide
RLRGPQRVFDLH

SEQ ID NO: 338 - protein, 9 aa, synthetic construct
  REGION 1..9: note = Description of Artificial Sequence: Synthetic peptide
GTPGPQGIA

SEQ ID NO: 339 - protein, 11 aa, synthetic construct
  REGION 1..11: note = Description of Artificial Sequence: Synthetic peptide
FDLHQNMGSVN

SEQ ID NO: 340 - protein, 6 aa, synthetic construct
  REGION 1..6: note = Description of Artificial Sequence: Synthetic peptide
GQRDVV

SEQ ID NO: 341 - protein, 10 aa, synthetic construct
  REGION 1..10: note = Description of Artificial Sequence: Synthetic peptide
LRAHAVDVNG

SEQ ID NO: 342 - protein, 12 aa, synthetic construct
  REGION 1..12: note = Description of Artificial Sequence: Synthetic peptide
TAGSCLRKFSTM

SEQ ID NO: 343 - protein, 10 aa, synthetic construct
  REGION 1..10: note = Description of Artificial Sequence: Synthetic peptide
LFSHAVSSNG

SEQ ID NO: 344 - protein, 6 aa, synthetic construct
  REGION 1..6: note = Description of Artificial Sequence: Synthetic peptide
KGHRGF

SEQ ID NO: 345 - protein, 15 aa, synthetic construct
  REGION 1..15: note = Description of Artificial Sequence: Synthetic peptide
TAGSCLRKFSTMFLF

SEQ ID NO: 346 - protein, 18 aa, synthetic construct
  REGION 1..18: note = Description of Artificial Sequence: Synthetic peptide
TAGSCLRKFSTMFLFCNI

SEQ ID NO: 347 - protein, 15 aa, synthetic construct
  REGION 1..15: note = Description of Artificial Sequence: Synthetic peptide
DLGTAGSCLRKFSTM

SEQ ID NO: 348 - protein, 18 aa, synthetic construct
  REGION 1..18: note = Description of Artificial Sequence: Synthetic peptide
HQQDLGTAGSCLRKFSTM

SEQ ID NO: 349 - protein, 10 aa, synthetic construct
  REGION 1..10: note = Description of Artificial Sequence: Synthetic peptide
RNIAEIIKDI

SEQ ID NO: 350 - protein, 11 aa, synthetic construct
  REGION 1..11: note = Description of Artificial Sequence: Synthetic peptide
SIGFRGDGQTC

SEQ ID NO: 351 - protein, 10 aa, synthetic construct
  REGION 1..10: note = Description of Artificial Sequence: Synthetic peptide
LNRQELFPFG

SEQ ID NO: 352 - protein, 17 aa, synthetic construct
  REGION 1..17: note = Description of Artificial Sequence: Synthetic peptide
RIQNLLKITNLRIKFVK

SEQ ID NO: 353 - protein, 16 aa, synthetic construct
  REGION 1..16: note = Description of Artificial Sequence: Synthetic peptide
KKQRFRHRNRKGYRSQ

SEQ ID NO: 354 - protein, 12 aa, synthetic construct
  REGION 1..12: note = Description of Artificial Sequence: Synthetic peptide
SINNTAVMQRLT

SEQ ID NO: 355 - protein, 9 aa, synthetic construct
  REGION 1..9: note = Description of Artificial Sequence: Synthetic peptide
FRHRNRKGY

SEQ ID NO: 356 - protein, 17 aa, synthetic construct
  REGION 1..17: note = Description of Artificial Sequence: Synthetic peptide
RYRVRVTPKEKTGPMKE

SEQ ID NO: 357 - protein, 13 aa, synthetic construct
  REGION 1..13: note = Description of Artificial Sequence: Synthetic peptide
SETTVKYIFRLHE

SEQ ID NO: 358 - protein, 21 aa, synthetic construct
  REGION 1..21: note = Description of Artificial Sequence: Synthetic peptide
GHRGPTGRPGKRGKQGQKGDS

SEQ ID NO: 359 - protein, 12 aa, synthetic construct
  REGION 1..12: note = Description of Artificial Sequence: Synthetic peptide
KAFDITYVRLKF

SEQ ID NO: 360 - protein, 15 aa, synthetic construct
  REGION 1..15: note = Description of Artificial Sequence: Synthetic peptide
GDLGRPGRKGRPGPP

SEQ ID NO: 361 - protein, 5 aa, synthetic construct
  REGION 1..5: note = Description of Artificial Sequence: Synthetic peptide
YIGSR

SEQ ID NO: 362 - protein, 14 aa, synthetic construct
  REGION 1..14: note = Description of Artificial Sequence: Synthetic peptide
RGEFYFDLRLKGDK

SEQ ID NO: 363 - protein, 12 aa, synthetic construct
  REGION 1..12: note = Description of Artificial Sequence: Synthetic peptide
LAGSCLARFSTM

SEQ ID NO: 364 - protein, 12 aa, synthetic construct
  REGION 1..12: note = Description of Artificial Sequence: Synthetic peptide
LALFLSNGHFVA

SEQ ID NO: 365 - protein, 12 aa, synthetic construct
  REGION 1..12: note = Description of Artificial Sequence: Synthetic peptide
ISRCQVCMKKRH

SEQ ID NO: 366 - protein, 12 aa, synthetic construct
  REGION 1..12: note = Description of Artificial Sequence: Synthetic peptide
PGRWHKVSVRWE

SEQ ID NO: 367 - protein, 15 aa, synthetic construct
  REGION 1..15: note = Description of Artificial Sequence: Synthetic peptide
TDIPPCPHGWISLWK

SEQ ID NO: 368 - protein, 12 aa, synthetic construct
  REGION 1..12: note = Description of Artificial Sequence: Synthetic peptide
VRWGMQQIQLVV

SEQ ID NO: 369 - protein, 15 aa, synthetic construct
  REGION 1..15: note = Description of Artificial Sequence: Synthetic peptide
TAIPSCPEGTVPLYS

SEQ ID NO: 370 - protein, 12 aa, synthetic construct
  REGION 1..12: note = Description of Artificial Sequence: Synthetic peptide
KMPYVSLELEMR

SEQ ID NO: 371 - protein, 13 aa, synthetic construct
  REGION 1..13: note = Description of Artificial Sequence: Synthetic peptide
GPAGKDGEAGAQG

SEQ ID NO: 372 - protein, 12 aa, synthetic construct
  REGION 1..12: note = Description of Artificial Sequence: Synthetic peptide
VLLQANDGAGEF

SEQ ID NO: 373 - protein, 6 aa, synthetic construct
  REGION 1..6: note = Description of Artificial Sequence: Synthetic peptide
GLPGER

SEQ ID NO: 374 - protein, 12 aa, synthetic construct
  REGION 1..12: note = Description of Artificial Sequence: Synthetic peptide
DGRWHRVAVIMG

SEQ ID NO: 375 - protein, 12 aa, synthetic construct
  REGION 1..12: note = Description of Artificial Sequence: Synthetic peptide
LAGSCLPVFSTL

SEQ ID NO: 376 - protein, 12 aa, synthetic construct
  REGION 1..12: note = Description of Artificial Sequence: Synthetic peptide
APVNVTASVQIQ

SEQ ID NO: 377 - protein, 12 aa, synthetic construct
  REGION 1..12: note = Description of Artificial Sequence: Synthetic peptide
TAGSCLRRFSTM

SEQ ID NO: 378 - protein, 12 aa, synthetic construct
  REGION 1..12: note = Description of Artificial Sequence: Synthetic peptide
KQGKALTQRHAK

SEQ ID NO: 379 - protein, 9 aa, synthetic construct
  REGION 1..9: note = Description of Artificial Sequence: Synthetic peptide
TAGSCLRKF

SEQ ID NO: 380 - protein, 7 aa, synthetic construct
  REGION 1..7: note = Description of Artificial Sequence: Synthetic peptide
RYVVLPR

SEQ ID NO: 381 - protein, 6 aa, synthetic construct
  REGION 1..6: note = Description of Artificial Sequence: Synthetic peptide
TAGSCL

SEQ ID NO: 382 - protein, 14 aa, synthetic construct
  REGION 1..14: note = Description of Artificial Sequence: Synthetic peptide
SPYTFIDSLVLMPY

SEQ ID NO: 383 - skipped

SEQ ID NO: 384 - protein, 5 aa, synthetic construct
  REGION 1..5: note = Description of Artificial Sequence: Synthetic peptide
PDSGR

SEQ ID NO: 385 - protein, 12 aa, synthetic construct
  REGION 1..12: note = Description of Artificial Sequence: Synthetic peptide
QQNLGSVNVSTG

SEQ ID NO: 386 - protein, 12 aa, synthetic construct
  REGION 1..12: note = Description of Artificial Sequence: Synthetic peptide
SRATAQKVSRRS

SEQ ID NO: 387 - protein, 8 aa, synthetic construct
  REGION 1..8: note = Description of Artificial Sequence: Synthetic peptide
DPGYIGSR

SEQ ID NO: 388 - protein, 12 aa, synthetic construct
  REGION 1..12: note = Description of Artificial Sequence: Synthetic peptide
GSLSSHLEFVGI

SEQ ID NO: 389 - protein, 12 aa, synthetic construct
  REGION 1..12: note = Description of Artificial Sequence: Synthetic peptide
VILQQSAADIAR

SEQ ID NO: 390 - protein, 12 aa, synthetic construct
  REGION 1..12: note = Description of Artificial Sequence: Synthetic peptide
RNRLHLSMLVRP

SEQ ID NO: 391 - protein, 12 aa, synthetic construct
  REGION 1..12: note = Description of Artificial Sequence: Synthetic peptide
KDISEKVAVYST

SEQ ID NO: 392 - protein, 13 aa, synthetic construct
  REGION 1..13: note = Description of Artificial Sequence: Synthetic peptide
APMSGRSPSLVLK

SEQ ID NO: 393 - protein, 6 aa, synthetic construct
  REGION 1..6: note = Description of Artificial Sequence: Synthetic peptide
LGTIPG

SEQ ID NO: 394 - protein, 12 aa, synthetic construct
  REGION 1..12: note = Description of Artificial Sequence: Synthetic peptide
AFGVLALWGTRV

SEQ ID NO: 395 - protein, 13 aa, synthetic construct
  REGION 1..13: note = Description of Artificial Sequence: Synthetic peptide
TDIRVTLNRLNTF

SEQ ID NO: 396 - protein, 12 aa, synthetic construct
  REGION 1..12: note = Description of Artificial Sequence: Synthetic peptide
IENVVTTFAPNR

SEQ ID NO: 397 - protein, 12 aa, synthetic construct
  REGION 1..12: note = Description of Artificial Sequence: Synthetic peptide
AFSTLEGRPSAY

SEQ ID NO: 398 - protein, 12 aa, synthetic construct
  REGION 1..12: note = Description of Artificial Sequence: Synthetic peptide
LEAEFHFTHLIM

SEQ ID NO: 399 - protein, 12 aa, synthetic construct
  REGION 1..12: note = Description of Artificial Sequence: Synthetic peptide
TSAEAYNLLLRT

SEQ ID NO: 400 - protein, 12 aa, synthetic construct
  REGION 1..12: note = Description of Artificial Sequence: Synthetic peptide
HLIMTFKTFRPA

SEQ ID NO: 401 - protein, 12 aa, synthetic construct
  REGION 1..12: note = Description of Artificial Sequence: Synthetic peptide
LNRRYEQARNIS

SEQ ID NO: 402 - protein, 12 aa, synthetic construct
  REGION 1..12: note = Description of Artificial Sequence: Synthetic peptide
KTWGVYRYFAYD

SEQ ID NO: 403 - protein, 12 aa, synthetic construct
  REGION 1..12: note = Description of Artificial Sequence: Synthetic peptide
SLLSQLNNLLDQ

SEQ ID NO: 404 - protein, 12 aa, synthetic construct
  REGION 1..12: note = Description of Artificial Sequence: Synthetic peptide
TNLRIKFVKLHT

SEQ ID NO: 405 - protein, 10 aa, synthetic construct
  REGION 1..10: note = Description of Artificial Sequence: Synthetic peptide
RDIAEIIKDI

SEQ ID NO: 406 - protein, 8 aa, synthetic construct
  REGION 1..8: note = Description of Artificial Sequence: Synthetic peptide
KRLVTGQR

SEQ ID NO: 407 - protein, 6 aa, synthetic construct
  REGION 1..6: note = Description of Artificial Sequence: Synthetic peptide
SHAVSS

SEQ ID NO: 408 - protein, 12 aa, synthetic construct
  REGION 1..12: note = Description of Artificial Sequence: Synthetic peptide
GPGVVVVERQYI

SEQ ID NO: 409 - protein, 6 aa, synthetic construct
  REGION 1..6: note = Description of Artificial Sequence: Synthetic peptide
ADTPPV

SEQ ID NO: 410 - protein, 13 aa, synthetic construct
  REGION 1..13: note = Description of Artificial Sequence: Synthetic peptide
NEPKVLKSYYYAI

SEQ ID NO: 411 - protein, 10 aa, synthetic construct
  REGION 1..10: note = Description of Artificial Sequence: Synthetic peptide
LRAHAVDING

SEQ ID NO: 412 - protein, 12 aa, synthetic construct
  REGION 1..12: note = Description of Artificial Sequence: Synthetic peptide
YYAISDFAVGGR

SEQ ID NO: 413 - protein, 12 aa, synthetic construct
  REGION 1..12: note = Description of Artificial Sequence: Synthetic peptide
DSITKYFQMSLE

SEQ ID NO: 414 - protein, 13 aa, synthetic construct
  REGION 1..13: note = Description of Artificial Sequence: Synthetic peptide
LPFFNDRPWRRAT

SEQ ID NO: 415 - protein, 10 aa, synthetic construct
  REGION 1..10: note = Description of Artificial Sequence: Synthetic peptide
YTALIIATDN

SEQ ID NO: 416 - protein, 14 aa, synthetic construct
  REGION 1..14: note = Description of Artificial Sequence: Synthetic peptide
FDPELYRSTGHGGH

SEQ ID NO: 417 - protein, 10 aa, synthetic construct
  REGION 1..10: note = Description of Artificial Sequence: Synthetic peptide
VITVKDINDN

SEQ ID NO: 418 - protein, 12 aa, synthetic construct
  REGION 1..12: note = Description of Artificial Sequence: Synthetic peptide
TNAVGYSVYDIS

SEQ ID NO: 419 - protein, 10 aa, synthetic construct
  REGION 1..10: note = Description of Artificial Sequence: Synthetic peptide
GLDRESYPYY

SEQ ID NO: 420 - protein, 13 aa, synthetic construct
  REGION 1..13: note = Description of Artificial Sequence: Synthetic peptide
APVKFLGNQVLSY

SEQ ID NO: 421 - protein, 10 aa, synthetic construct
  REGION 1..10: note = Description of Artificial Sequence: Synthetic peptide
MKVSATDADD

SEQ ID NO: 422 - protein, 12 aa, synthetic construct
  REGION 1..12: note = Description of Artificial Sequence: Synthetic peptide
SFSFRVDRRDTR

SEQ ID NO: 423 - protein, 12 aa, synthetic construct
  REGION 1..12: note = Description of Artificial Sequence: Synthetic peptide
PQVTRGDVFTMP

SEQ ID NO: 424 - protein, 17 aa, synthetic construct
  REGION 1..17: note = Description of Artificial Sequence: Synthetic peptide
TWSKVGGHLRPGIVQSG

SEQ ID NO: 425 - protein, 12 aa, synthetic construct
  REGION 1..12: note = Description of Artificial Sequence: Synthetic peptide
KEAEREVTDLLR

SEQ ID NO: 426 - protein, 4 aa, synthetic construct
  REGION 1..4: note = Description of Artificial Sequence: Synthetic peptide
RGDV

SEQ ID NO: 427 - protein, 12 aa, synthetic construct
  REGION 1..12: note = Description of Artificial Sequence: Synthetic peptide
AAEPLKNIGILF

SEQ ID NO: 428 - protein, 11 aa, synthetic construct
  REGION 1..11: note = Description of Artificial Sequence: Synthetic peptide
FALWDAIIGEL

SEQ ID NO: 429 - protein, 6 aa, synthetic construct
  REGION 1..6: note = Description of Artificial Sequence: Synthetic peptide
VGVAPG

SEQ ID NO: 430 - protein, 12 aa, synthetic construct
  REGION 1..12: note = Description of Artificial Sequence: Synthetic peptide
LWPLLAVLAAVA

SEQ ID NO: 431 - protein, 5 aa, synthetic construct
  REGION 1..5: note = Description of Artificial Sequence: Synthetic peptide
PGVGV

SEQ ID NO: 432 - protein, 8 aa, synthetic construct
  REGION 1..8: note = Description of Artificial Sequence: Synthetic peptide
VFDNFVLK

SEQ ID NO: 433 - protein, 12 aa, synthetic construct
  REGION 1..12: note = Description of Artificial Sequence: Synthetic peptide
TSIKIRGTYSER

SEQ ID NO: 434 - protein, 10 aa, synthetic construct
  REGION 1..10: note = Description of Artificial Sequence: Synthetic peptide
TTSWSQCSKS

SEQ ID NO: 435 - protein, 6 aa, synthetic construct
  REGION 1..6: note = Description of Artificial Sequence: Synthetic peptide
DPETGV

SEQ ID NO: 436 - protein, 4 aa, synthetic construct
  REGION 1..4: note = Description of Artificial Sequence: Synthetic peptide
KRSR

SEQ ID NO: 437 - protein, 10 aa, synthetic construct
  REGION 1..10: note = Description of Artificial Sequence: Synthetic peptide
QGADTPPVGV

SEQ ID NO: 438 - protein, 7 aa, synthetic construct
  REGION 1..7: note = Description of Artificial Sequence: Synthetic peptide
SVVYGLR

SEQ ID NO: 439 - protein, 10 aa, synthetic construct
  REGION 1..10: note = Description of Artificial Sequence: Synthetic peptide
PLDREAIAKY

SEQ ID NO: 440 - protein, 10 aa, synthetic construct
  REGION 1..10: note = Description of Artificial Sequence: Synthetic peptide
DGRGDSVAYG

SEQ ID NO: 441 - protein, 5 aa, synthetic construct
  REGION 1..5: note = Description of Artificial Sequence: Synthetic peptide
HAVDI

SEQ ID NO: 442 - protein, 10 aa, synthetic construct
  REGION 1..10: note = Description of Artificial Sequence: Synthetic peptide
LALERKDHSG

SEQ ID NO: 443 - protein, 5 aa, synthetic construct
  REGION 1..5: note = Description of Artificial Sequence: Synthetic peptide
DQNDN

SEQ ID NO: 444 - protein, 19 aa, synthetic construct
  REGION 1..19: note = Description of Artificial Sequence: Synthetic peptide
YSMKKTTMKIIPFNRLTIG

SEQ ID NO: 445 - protein, 10 aa, synthetic construct
  REGION 1..10: note = Description of Artificial Sequence: Synthetic peptide
QDPELPDKNM

SEQ ID NO: 446 - protein, 4 aa, synthetic construct
  REGION 1..4: note = Description of Artificial Sequence: Synthetic peptide
RGDF

SEQ ID NO: 447 - protein, 10 aa, synthetic construct
  REGION 1..10: note = Description of Artificial Sequence: Synthetic peptide
LVVQAADLQG

SEQ ID NO: 448 - protein, 13 aa, synthetic construct
  REGION 1..13: note = Description of Artificial Sequence: Synthetic peptide
GVYYQGGTYSKAS

SEQ ID NO: 449 - protein, 10 aa, synthetic construct
  REGION 1..10: note = Description of Artificial Sequence: Synthetic peptide
NDDGGQFVVT

SEQ ID NO: 450 - protein, 12 aa, synthetic construct
  REGION 1..12: note = Description of Artificial Sequence: Synthetic peptide
TAGSCLRKFSCL

SEQ ID NO: 451 - protein, 9 aa, synthetic construct
  REGION 1..9: note = Description of Artificial Sequence: Synthetic peptide
YILHVAVTN

SEQ ID NO: 452 - protein, 19 aa, synthetic construct
  REGION 1..19: note = Description of Artificial Sequence: Synthetic peptide
CNYYSNSYSFWLASLNPER

SEQ ID NO: 453 - protein, 10 aa, synthetic construct
  REGION 1..10: note = Description of Artificial Sequence: Synthetic peptide
TYRIWRDTAN

SEQ ID NO: 454 - protein, 12 aa, synthetic construct
  REGION 1..12: note = Description of Artificial Sequence: Synthetic peptide
TGLSCLQRFTTM

SEQ ID NO: 455 - protein, 26 aa, synthetic construct
  REGION 1..26: note = Description of Artificial Sequence: Synthetic peptide
GFTCECSIGFRGDGQTCYGIVFWSEV

SEQ ID NO: 456 - protein, 12 aa, synthetic construct
  REGION 1..12: note = Description of Artificial Sequence: Synthetic peptide
HHLGGAKQAGDV

SEQ ID NO: 457 - protein, 21 aa, synthetic construct
  REGION 1..21: note = Description of Artificial Sequence: Synthetic peptide
SCLPGFSGDGRACRDVDECGH

SEQ ID NO: 458 - protein, 30 aa, synthetic construct
  REGION 1..30: note = Description of Artificial Sequence: Synthetic polypeptide
MAPRPSLAKKQRFRHRNRKGYRSQRGHSRG

SEQ ID NO: 459 - protein, 16 aa, synthetic construct
  REGION 1..16: note = Description of Artificial Sequence: Synthetic peptide
KKQKFRHRNRKGYRSQ

SEQ ID NO: 460 - protein, 15 aa, synthetic construct
  REGION 1..15: note = Description of Artificial Sequence: Synthetic peptide
KKQKFKHRNRKGYRS

SEQ ID NO: 461 - protein, 16 aa, synthetic construct
  REGION 1..16: note = Description of Artificial Sequence: Synthetic peptide
KKQKFRRRNRKGYRSH

SEQ ID NO: 462 - protein, 15 aa, synthetic construct
  REGION 1..15: note = Description of Artificial Sequence: Synthetic peptide
TAIPPCPHGWISLWK

SEQ ID NO: 463 - protein, 15 aa, synthetic construct
  REGION 1..15: note = Description of Artificial Sequence: Synthetic peptide
KKQKSRHRSRKRYRS

SEQ ID NO: 464 - protein, 15 aa, synthetic construct
  REGION 1..15: note = Description of Artificial Sequence: Synthetic peptide
KKQKSRRRSRKGYRS

SEQ ID NO: 465 - protein, 7 aa, synthetic construct
  REGION 1..7: note = Description of Artificial Sequence: Synthetic peptide
ISRCTVC

SEQ ID NO: 466 - protein, 12 aa, synthetic construct
  REGION 1..12: note = Description of Artificial Sequence: Synthetic peptide
ISRCQVCMKRRH

SEQ ID NO: 467 - protein, 7 aa, synthetic construct
  REGION 1..7: note = Description of Artificial Sequence: Synthetic peptide
VSRCTVC

SEQ ID NO: 468 - protein, 15 aa, synthetic construct
  REGION 1..15: note = Description of Artificial Sequence: Synthetic peptide
TDIPPCPQGWISLWK

SEQ ID NO: 469 - protein, 21 aa, synthetic construct
  REGION 1..21: note = Description of Artificial Sequence: Synthetic peptide
TVKAGELEKIISRCQVMKKRH

SEQ ID NO: 470 - protein, 15 aa, synthetic construct
  REGION 1..15: note = Description of Artificial Sequence: Synthetic peptide
TDIPSCPHGWISLWK

SEQ ID NO: 471 - protein, 15 aa, synthetic construct
  REGION 1..15: note = Description of Artificial Sequence: Synthetic peptide
TDIPPCPAGWISLWK

SEQ ID NO: 472 - protein, 15 aa, synthetic construct
  REGION 1..15: note = Description of Artificial Sequence: Synthetic peptide
TEIPPCPQGWISLWK

SEQ ID NO: 473 - protein, 15 aa, synthetic construct
  REGION 1..15: note = Description of Artificial Sequence: Synthetic peptide
TDVPPCPQGWISLWK

SEQ ID NO: 474 - protein, 13 aa, synthetic construct
  REGION 1..13: note = Description of Artificial Sequence: Synthetic peptide
RLVSYNGILFFLK

SEQ ID NO: 475 - protein, 13 aa, synthetic construct
  REGION 1..13: note = Description of Artificial Sequence: Synthetic peptide
RLVSYSGVIFFLK

SEQ ID NO: 476 - protein, 12 aa, synthetic construct
  REGION 1..12: note = Description of Artificial Sequence: Synthetic peptide
RLVSYNGILFFL

SEQ ID NO: 477 - protein, 13 aa, synthetic construct
  REGION 1..13: note = Description of Artificial Sequence: Synthetic peptide
RLVSYSGIIFFLK

SEQ ID NO: 478 - protein, 20 aa, synthetic construct
  REGION 1..20: note = Description of Artificial Sequence: Synthetic peptide
RFEQELRLVSYSGVLFFLKQ

SEQ ID NO: 479 - protein, 13 aa, synthetic construct
  REGION 1..13: note = Description of Artificial Sequence: Synthetic peptide
RLVSYNGIIFFLK

SEQ ID NO: 480 - protein, 30 aa, synthetic construct
  REGION 1..30: note = Description of Artificial Sequence: Synthetic polypeptide
DPAFKIEDPYSPRIQNLLKITNLRIKFVKL

SEQ ID NO: 481 - protein, 20 aa, synthetic construct
  REGION 1..20: note = Description of Artificial Sequence: Synthetic peptide
TKRFEQELRLVSYSGVLFFL

SEQ ID NO: 482 - protein, 10 aa, synthetic construct
  REGION 1..10: note = Description of Artificial Sequence: Synthetic peptide
GGRLKYSVAF

SEQ ID NO: 483 - protein, 12 aa, synthetic construct
  REGION 1..12: note = Description of Artificial Sequence: Synthetic peptide
GGFLRYTVSYDI

SEQ ID NO: 484 - protein, 12 aa, synthetic construct
  REGION 1..12: note = Description of Artificial Sequence: Synthetic peptide
GGFLKYTVSYDV

SEQ ID NO: 485 - protein, 22 aa, synthetic construct
  REGION 1..22: note = Description of Artificial Sequence: Synthetic peptide
LGNKLTAFGGFLKYTVSYDIPV

SEQ ID NO: 486 - protein, 12 aa, synthetic construct
  REGION 1..12: note = Description of Artificial Sequence: Synthetic peptide
GGYLKYTVSYDI

SEQ ID NO: 487 - protein, 13 aa, synthetic construct
  REGION 1..13: note = Description of Artificial Sequence: Synthetic peptide
GEIFFDMRLKGDK

SEQ ID NO: 488 - protein, 13 aa, synthetic construct
  REGION 1..13: note = Description of Artificial Sequence: Synthetic peptide
GEIYFDLRLKGDK

SEQ ID NO: 489 - protein, 13 aa, synthetic construct
  REGION 1..13: note = Description of Artificial Sequence: Synthetic peptide
GEIYLDMRLKGDK

SEQ ID NO: 490 - protein, 30 aa, synthetic construct
  REGION 1..30: note = Description of Artificial Sequence: Synthetic polypeptide
IGQPGAKGEPGEFYFDLRLKGDKGDPGFPG

SEQ ID NO: 491 - protein, 13 aa, synthetic construct
  REGION 1..13: note = Description of Artificial Sequence: Synthetic peptide
GEVFFDMRLKGDK

SEQ ID NO: 492 - protein, 12 aa, synthetic construct
  REGION 1..12: note = Description of Artificial Sequence: Synthetic peptide
LAGSCLPIFSTL

SEQ ID NO: 493 - protein, 30 aa, synthetic construct
  REGION 1..30: note = Description of Artificial Sequence: Synthetic polypeptide
AHNQDLGLAGSCLARFSTMPFLYCNPGDIC

SEQ ID NO: 494 - protein, 30 aa, synthetic construct
  REGION 1..30: note = Description of Artificial Sequence: Synthetic polypeptide
QEKAHNQDLGLAGSCLPVFSTLPFAYCNIH

SEQ ID NO: 495 - protein, 12 aa, synthetic construct
  REGION 1..12: note = Description of Artificial Sequence: Synthetic peptide
LAGSCLPVFSTM

SEQ ID NO: 496 - protein, 30 aa, synthetic construct
  REGION 1..30: note = Description of Artificial Sequence: Synthetic polypeptide
GNKRAHGQDLGTAGSCLRRFSTMPFMFCNI

SEQ ID NO: 497 - protein, 21 aa, synthetic construct
  REGION 1..21: note = Description of Artificial Sequence: Synthetic peptide
RAHGQDLGTAGSCLRRFSTMP

SEQ ID NO: 498 - protein, 12 aa, synthetic construct
  REGION 1..12: note = Description of Artificial Sequence: Synthetic peptide
RKRLQVQLNIRT

SEQ ID NO: 499 - protein, 30 aa, synthetic construct
  REGION 1..30: note = Description of Artificial Sequence: Synthetic polypeptide
HLVLPLQQSDVRKRLQVQLSIRTFASSGLI

SEQ ID NO: 500 - protein, 12 aa, synthetic construct
  REGION 1..12: note = Description of Artificial Sequence: Synthetic peptide
RKRLSVQLRIRT

SEQ ID NO: 501 - protein, 15 aa, synthetic construct
  REGION 1..15: note = Description of Artificial Sequence: Synthetic peptide
DLGTAGSCLRRFSTM

SEQ ID NO: 502 - protein, 10 aa, synthetic construct
  REGION 1..10: note = Description of Artificial Sequence: Synthetic peptide
RNIAEIIKDI

SEQ ID NO: 503 - protein, 24 aa, synthetic construct
  REGION 1..24: note = Description of Artificial Sequence: Synthetic peptide
TAGSCLRKFSTMRRRRRRRRRRRR

SEQ ID NO: 504 - protein, 17 aa, synthetic construct
  REGION 1..17: note = Description of Artificial Sequence: Synthetic peptide
FTLTGLLGTLVTMGLLT

SEQ ID NO: 505 - protein, 7 aa, synthetic construct
  REGION 1..7: note = Description of Artificial Sequence: Synthetic peptide
APYKAWK

SEQ ID NO: 506 - protein, 23 aa, synthetic construct
  REGION 1..23: note = Description of Artificial Sequence: Synthetic peptide
STSKTNRGDDSNWSKRVTNNKPS

SEQ ID NO: 507 - protein, 23 aa, synthetic construct
  REGION 1..23: note = Description of Artificial Sequence: Synthetic peptide
STSKRKRGDDSNWSKRVTKKKPS

SEQ ID NO: 508 - protein, 23 aa, synthetic construct
  REGION 1..23: note = Description of Artificial Sequence: Synthetic peptide
STSKRKRGDDSNWSKRVSKKKPS

SEQ ID NO: 509 - protein, 23 aa, synthetic construct
  REGION 1..23: note = Description of Artificial Sequence: Synthetic peptide
STSKRKRGDDANWSKRVTKKKPS

SEQ ID NO: 510 - protein, 26 aa, synthetic construct
  REGION 1..26: note = Description of Artificial Sequence: Synthetic peptide
PLAGSKRKRADEVAWSKRGTKKKPER

SEQ ID NO: 511 - protein, 39 aa, synthetic construct
  REGION 1..39: note = Description of Artificial Sequence: Synthetic polypeptide
PLAGSKRKRADEVAWSKRGTKKKPERTSAARAGPSRRIR

SEQ ID NO: 512 - protein, 24 aa, synthetic construct
  REGION 1..24: note = Description of Artificial Sequence: Synthetic peptide
STSKRKRGDDANWSKRTTKKKPSS

SEQ ID NO: 513 - protein, 39 aa, synthetic construct
  REGION 1..39: note = Description of Artificial Sequence: Synthetic polypeptide
STSKRKRGDDANWSKRTTKKKPSSAGLKRAGSKADRPSL

SEQ ID NO: 514 - protein, 26 aa, synthetic construct
  REGION 1..26: note = Description of Artificial Sequence: Synthetic peptide
PTTAGKRKRSDDAAWSKRARPKAGRT

SEQ ID NO: 515 - protein, 39 aa, synthetic construct
  REGION 1..39: note = Description of Artificial Sequence: Synthetic polypeptide
PTTAGKRKRSDDAAWSKRARPKAGRTSAARPGTSVRRIR

SEQ ID NO: 516 - protein, 26 aa, synthetic construct
  REGION 1..26: note = Description of Artificial Sequence: Synthetic peptide
SSSLGKRKRSDEGAWSKGKSKKKAMR

SEQ ID NO: 517 - protein, 39 aa, synthetic construct
  REGION 1..39: note = Description of Artificial Sequence: Synthetic polypeptide
SSSLGKRKRSDEGAWSKGKSKKKAMRGSSSRRPGPVRGP

SEQ ID NO: 518 - protein, 25 aa, synthetic construct
  REGION 1..25: note = Description of Artificial Sequence: Synthetic peptide
PTTAGKRKRTDDAAWSKRARPKAGR

SEQ ID NO: 519 - protein, 39 aa, synthetic construct
  REGION 1..39: note = Description of Artificial Sequence: Synthetic polypeptide
PTTAGKRKRTDDAAWSKRARPKAGRTSAARPGTAVRRVR

SEQ ID NO: 520 - protein, 30 aa, synthetic construct
  REGION 1..30: note = Description of Artificial Sequence: Synthetic polypeptide
PATAGKRKRSDDAAWSKRARPKAGRTSAAR

SEQ ID NO: 521 - protein, 39 aa, synthetic construct
  REGION 1..39: note = Description of Artificial Sequence: Synthetic polypeptide
PATAGKRKRSDDAAWSKRARPKAGRTSAARPGTSVRRIR

SEQ ID NO: 522 - protein, 25 aa, synthetic construct
  REGION 1..25: note = Description of Artificial Sequence: Synthetic peptide
SSSLGKRKRSNGGDWSKRSAKKKPA

SEQ ID NO: 523 - protein, 39 aa, synthetic construct
  REGION 1..39: note = Description of Artificial Sequence: Synthetic polypeptide
SSSLGKRKRSNGGDWSKRSAKKKPAGTPSRRAGPGRGPR

SEQ ID NO: 524 - protein, 26 aa, synthetic construct
  REGION 1..26: note = Description of Artificial Sequence: Synthetic peptide
SSSLGKRKRSDEGAWSKGKSKKKAMR

SEQ ID NO: 525 - protein, 39 aa, synthetic construct; identical to SEQ ID NO: 517
  REGION 1..39: note = Description of Artificial Sequence: Synthetic polypeptide

SEQ ID NO: 526 - protein, 24 aa, synthetic construct
  REGION 1..24: note = Description of Artificial Sequence: Synthetic peptide
STSKRKRGDDANWNKRPTKKKPSS

SEQ ID NO: 527 - protein, 39 aa, synthetic construct
  REGION 1..39: note = Description of Artificial Sequence: Synthetic polypeptide
STSKRKRGDDANWNKRPTKKKPSSAGLKKAGSKAERPSL

SEQ ID NO: 528 - protein, 24 aa, synthetic construct
  REGION 1..24: note = Description of Artificial Sequence: Synthetic peptide
SGALKRKRSDEVAWSRRRPVKKPV

SEQ ID NO: 529 - protein, 39 aa, synthetic construct
  REGION 1..39: note = Description of Artificial Sequence: Synthetic polypeptide
SGALKRKRSDEVAWSRRRPVKKPVRRAPPPRAGPSVRRG

SEQ ID NO: 530 - protein, 25 aa, synthetic construct
  REGION 1..25: note = Description of Artificial Sequence: Synthetic peptide
SGALKRKRSDEVAWSRRKPAKKPAR

SEQ ID NO: 531 - protein, 39 aa, synthetic construct
  REGION 1..39: note = Description of Artificial Sequence: Synthetic polypeptide
SGALKRKRSDEVAWSRRKPAKKPARQPPPPRAGPSVRRG

SEQ ID NO: 532 - protein, 25 aa, synthetic construct
  REGION 1..25: note = Description of Artificial Sequence: Synthetic peptide
AGALKRKRSDEVAWSRRKPAKKPAR

SEQ ID NO: 533 - protein, 39 aa, synthetic construct
  REGION 1..39: note = Description of Artificial Sequence: Synthetic polypeptide
AGALKRKRSDEVAWSRRKPAKKPARAPPPRAGPSVRRGL

SEQ ID NO: 534 - protein, 243 aa, synthetic construct
  REGION 1..243: note = Description of Artificial Sequence: Synthetic polypeptide
STSKRKRGDDSNWSKRVTKKKPSSAGLKRAGSKADRPSLQIQTLQHAGTTMITVPSGGVCDLINTYARGSDEGNRHTSETLTYKIAIDYHFVADAAACRYSNTGTGVMWLVYDTTPGGQAPTPQTIFSYPDTLKAWPATWKVSRELCHRFVVKRRWLFNMETDGRIGSDIPPSNASWKPCKRNIYFHKFTSGLGVRTQWKNVTDGGVGAIQRGALYMVIAPGNGLTFTAHGQTRLYFKSVGNQ

SEQ ID NO: 535 - protein, 100 aa, synthetic construct
  REGION 1..100: note = Description of Artificial Sequence: Synthetic polypeptide
DPQNALYYQPRVPTAAPTSGGVPWSRVGEVAILSFVALICFYLLYLWVLRDLILVLKARQGRSTEELIFGGQAVDRSNPIPNIPAPPSQGNPGPFVPGTG

SEQ ID NO: 536 - protein, 292 aa, synthetic construct
  REGION 1..292: note = Description of Artificial Sequence: Synthetic polypeptide
GSQLVPPPSAFNYIESQRDEFQLSHDLTEIVLQFPSTASQITARLSRSCMKIDHCVIEYRQQVPINASGTVIVEIHDKRMTDNESLQASWTFPIRCNIDLHYFSSSFFSLKDPIPWKLYYRVSDSNVHQMTHFAKFKGKLKLSSAKHSVDIPFRAPTVKILAKQFSEKDIDFWHVGYGKWERRLVKSASSSRFGLRGPIEINPGESWATKSAIVTPNRNADLDIEEELLPYRELNRLGTNILDPGESASIVGIQRSQSNITMSMSQLNELVRSTVHECIKTSCIPSTPKSLS

SEQ ID NO: 537 - protein, 22 aa, synthetic construct
  REGION 1..22: note = Description of Artificial Sequence: Synthetic peptide
RTGVKRSYGAARGDDRRRPNVV

SEQ ID NO: 538 - protein, 19 aa, synthetic construct
  REGION 1..19: note = Description of Artificial Sequence: Synthetic peptide
SYVKTVPNRTRTYIKLRVR

SEQ ID NO: 539 - protein, 255 aa, synthetic construct
  REGION 1..255: note = Description of Artificial Sequence: Synthetic polypeptide
MYSTSNRRGRSQTQRGSHVRRTGVKRSYGAARGDDRRRPNVVSKTQVEPRMTIQRVQENQFGPEFVLSQNSALSTFVTYPSYVKTVPNRTRTYIKLKRVRFKGTLKIERGQGDTIMDGPSSNIEGVFSMVIVVDRKPHVSQSGRLHTFDELFGARIHCHGNLSVVPALKDRYYIRHVTKRVVSLEKDTLLIDLHGTTQLSNKRYNCWASFSDLERDCNGVYGNITKNALLVYYCWLSDAQSKASTYVSFELDYLG

SEQ ID NO: 540 - protein, 30 aa, synthetic construct
  REGION 1..30: note = Description of Artificial Sequence: Synthetic polypeptide
RRRRRRRRRRRRVDYGKWERKPIRCASMSR

SEQ ID NO: 541 - protein, 24 aa, synthetic construct
  REGION 1..24: note = Description of Artificial Sequence: Synthetic peptide
RRRRRRRRRRRRGKWERKPIRCAS

SEQ ID NO: 542 - protein, 28 aa, synthetic construct
  REGION 1..28: note = Description of Artificial Sequence: Synthetic peptide
KKKKKKKKKKKKKKKKGKWERKPIRCAS

SEQ ID NO: 543 - protein, 40 aa, synthetic construct
  REGION 1..40: note = Description of Artificial Sequence: Synthetic polypeptide
RRRRRRRRRRRRVDFSHVDYGKWERKPIRCASMSRLGLRG

SEQ ID NO: 544 - protein, 38 aa, synthetic construct
  REGION 1..38: note = Description of Artificial Sequence: Synthetic polypeptide
GVKRSYGAARGDDRRRPNVVAPYKAWRRRRRRRRRRRR

SEQ ID NO: 545 - protein, 38 aa, synthetic construct
  REGION 1..38: note = Description of Artificial Sequence: Synthetic polypeptide
KSVPNRTRTYIKLKRLRFKGAPYKAWRRRRRRRRRRRR

SEQ ID NO: 546 - protein, 34 aa, synthetic construct
  REGION 1..34: note = Description of Artificial Sequence: Synthetic polypeptide
RTGVKRSYGAARGDDRRRPNVVRRRRRRRRRRRR

SEQ ID NO: 547 - protein, 32 aa, synthetic construct
  REGION 1..32: note = Description of Artificial Sequence: Synthetic polypeptide
SYVKTVPNRTRTYIKGGGGGRRRRRRRRRRRR

SEQ ID NO: 548 - protein, 50 aa, synthetic construct
  REGION 1..50: note = Description of Artificial Sequence: Synthetic polypeptide
VDIPFRAPTIKILSKQFTEDDIDFWHVGYGKWERKLVRPASLSGRRGLRR

SEQ ID NO: 549 - protein, 29 aa, synthetic construct
  REGION 1..29: note = Description of Artificial Sequence: Synthetic peptide
IDFWHVGYGKWERKLVRPASLSGRRGLRR

SEQ ID NO: 550 - protein, 31 aa, synthetic construct
  REGION 1..31: note = Description of Artificial Sequence: Synthetic polypeptide
IDFWSVEKGETRRRLLNPTPHAHSPRPIAHR

SEQ ID NO: 551 - protein, 28 aa, synthetic construct
  REGION 1..28: note = Description of Artificial Sequence: Synthetic peptide
IDFSHVGYGKWERKMIRSASISRLGLHN

SEQ ID NO: 552 - protein, 28 aa, synthetic construct
  REGION 1..28: note = Description of Artificial Sequence: Synthetic peptide
VDFSHVGYGKWERKLIRSASTVKYGLPS

SEQ ID NO: 553 - protein, 28 aa, synthetic construct
  REGION 1..28: note = Description of Artificial Sequence: Synthetic peptide
IDFSHVDYGKVERKLVKCESSSRLGLHS

SEQ ID NO: 554 - protein, 29 aa, synthetic construct
  REGION 1..29: note = Description of Artificial Sequence: Synthetic peptide
IDFWSVGRKAQQRKLVQGPSLIGSRSMRY

SEQ ID NO: 555 - protein, 31 aa, synthetic construct
  REGION 1..31: note = Description of Artificial Sequence: Synthetic polypeptide
IDFWSVGSKPQTRRLVDGSRLIGHSSRSLRV

SEQ ID NO: 556 - protein, 31 aa, synthetic construct
  REGION 1..31: note = Description of Artificial Sequence: Synthetic polypeptide
IDFWSVERGETRRRLLNPTPSAGSNRALSKR

SEQ ID NO: 557 - protein, 31 aa, synthetic construct
  REGION 1..31: note = Description of Artificial Sequence: Synthetic polypeptide
VDFWSVGKPKPIRRLIQNDPGTDYDTGPKYR

SEQ ID NO: 558 - protein, 31 aa, synthetic construct
  REGION 1..31: note = Description of Artificial Sequence: Synthetic polypeptide
VDFWSVEKPKPIRRLLNPGPNQGPYPNTGHR

SEQ ID NO: 559 - protein, 28 aa, synthetic construct
  REGION 1..28: note = Description of Artificial Sequence: Synthetic peptide
VDFSHVDYGKWERKLIRSASTSRYGLRS

SEQ ID NO: 560 - protein, 28 aa, synthetic construct
  REGION 1..28: note = Description of Artificial Sequence: Synthetic peptide
VDFSHVDYGKWERKTLRSRSLSRIGLTG

SEQ ID NO: 561 - protein, 28 aa, synthetic construct
  REGION 1..28: note = Description of Artificial Sequence: Synthetic peptide
IDFWHVGYGKWERRLVKSASSSRFGIRG

SEQ ID NO: 562 - protein, 28 aa, synthetic construct
  REGION 1..28: note = Description of Artificial Sequence: Synthetic peptide
VDFFHVDYGRWERKHIRCASMSRVGLRG

SEQ ID NO: 563 - protein, 28 aa, synthetic construct
  REGION 1..28: note = Description of Artificial Sequence: Synthetic peptide
GTFQHVDYGKWERKPIRCQSMSRVGYRR

SEQ ID NO: 564 - protein, 17 aa, synthetic construct
  REGION 1..17: note = Description of Artificial Sequence: Synthetic peptide
VGYGKWERKLVRPASLS

SEQ ID NO: 565 - protein, 17 aa, synthetic construct
  REGION 1..17: note = Description of Artificial Sequence: Synthetic peptide
VEKGETRRRLLNPTPHA

SEQ ID NO: 566 - protein, 17 aa, synthetic construct
  REGION 1..17: note = Description of Artificial Sequence: Synthetic peptide
VGYGKWERKLIRSASTV

SEQ ID NO: 567 - protein, 17 aa, synthetic construct
  REGION 1..17: note = Description of Artificial Sequence: Synthetic peptide
VEKPKPIRRLLNPGPNQ

SEQ ID NO: 568 - protein, 17 aa, synthetic construct
  REGION 1..17: note = Description of Artificial Sequence: Synthetic peptide
VDYGKWERKLIRSASTS

SEQ ID NO: 569 - protein, 17 aa, synthetic construct
  REGION 1..17: note = Description of Artificial Sequence: Synthetic peptide
VDYGKWERKTLRSRSLS

SEQ ID NO: 570 - protein, 17 aa, synthetic construct
  REGION 1..17: note = Description of Artificial Sequence: Synthetic peptide
VGYGKWERRLVKSASSS

SEQ ID NO: 571 - protein, 17 aa, synthetic construct
  REGION 1..17: note = Description of Artificial Sequence: Synthetic peptide
VDYGRWERKHIRCASMS

SEQ ID NO: 572 - protein, 17 aa, synthetic construct
  REGION 1..17: note = Description of Artificial Sequence: Synthetic peptide
VERPKPIRRLLTPTPGC

SEQ ID NO: 573 - protein, 47 aa, synthetic construct
  REGION 1..47: note = Description of Artificial Sequence: Synthetic polypeptide
PFRAPTIKILSKQFTEDDIDFWHVGYGKWERKLVRPASLSGRRGLRR

SEQ ID NO: 574 - protein, 44 aa, synthetic construct
  REGION 1..44: note = Description of Artificial Sequence: Synthetic polypeptide
PFRAPTVKILSKQFTDKDIDFSHVGYGKWERKMIRSASISRLGL

SEQ ID NO: 575 - protein, 46 aa, synthetic construct
  REGION 1..46: note = Description of Artificial Sequence: Synthetic polypeptide
DIAFRAPTVKILSKQFTDRDVDFSHVGYGKWERKLIRSASTVKYGL

SEQ ID NO: 576 - protein, 49 aa, synthetic construct
  REGION 1..49: note = Description of Artificial Sequence: Synthetic polypeptide
DIRFKPPTINILSKDYTADCVDFWSVEKPKPIRRLLNPGPNQGPYPNTG

SEQ ID NO: 577 - protein, 46 aa, synthetic construct
  REGION 1..46: note = Description of Artificial Sequence: Synthetic polypeptide
DIPFRAPTVKIHSKQFSHRDVDFSHVDYGKWERKTLRSRSLSRIGL

SEQ ID NO: 578 - protein, 46 aa, synthetic construct
  REGION 1..46: note = Description of Artificial Sequence: Synthetic polypeptide
DIPFRAPTVKILAKQFSEKDIDFWHVGYGKWERRLVKSASSSRFGI

SEQ ID NO: 579 - protein, 46 aa, synthetic construct
  REGION 1..46: note = Description of Artificial Sequence: Synthetic polypeptide
DIPFRAPTVKILSKQFTDKDVDFFHVDYGRWERKHIRCASMSRVGL

SEQ ID NO: 580 - protein, 43 aa, synthetic construct
  REGION 1..43: note = Description of Artificial Sequence: Synthetic polypeptide
DIKYKPPTIKILSKDYTADCVDFWSVERPKPIRRLLTPTPGCG

SEQ ID NO: 581 - protein, 47 aa, synthetic construct
  REGION 1..47: note = Description of Artificial Sequence: Synthetic polypeptide
ARTKQTARKSTGGKAPRKQLATKAARKSAPATGGVKKPHRYRPGTVA

SEQ ID NO: 582 - protein, 34 aa, synthetic construct
  REGION 1..34: note = Description of Artificial Sequence: Synthetic polypeptide
SGRGKGGKGLGKGGAKRHRKVLRDNIQGITKPAI

SEQ ID NO: 583 - protein, 10 aa, synthetic construct
  REGION 1..10: note = Description of Artificial Sequence: Synthetic peptide
GRKKRRQRRR

SEQ ID NO: 584 - protein, 58 aa, synthetic construct
  REPEAT 38..57: note = 0-20 of the lysine residues may be absent
GYGPPKKKRKVEAPYKAKKKKKKKKKKKKKKKKKKKKKKKKKKKKKKKKKKKKKKKKK